SEQUENCE LISTING (138 sequences)

SEQ ID NO: 1 - protein, 1125 aa, Unknown
  Description of Unknown Candidatus Katanobacteria sequence
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 2 - DNA, 3380 bp, Unknown
  Description of Unknown Candidatus Katanobacteria sequence
atgcgcaaaaaattgtttaagggttacattttacataataagaggcttgtatatacaggtaaagctgcaatacgttctattaaatatccattagtcgctccaaataaaacagccttaaacaatttatcagaaaagataatttatgattatgagcatttattcggacctttaaatgtggctagctatgcaagaaattcaaacaggtacagccttgtggatttttggatagatagcttgcgagcaggtgtaatttggcaaagcaaaagtacttcgctaattgatttgataagtaagctagaaggatctaaatccccatcagaaaagatatttgaacaaatagattttgagctaaaaaataagttggataaagagcaattcaaagatattattcttcttaatacaggaattcgttctagcagtaatgttcgcagtttgagggggcgctttctaaagtgttttaaagaggaatttagagataccgaagaggttatcgcctgtgtagataaatggagcaaggaccttatcgtagagggtaaaagtatactagtgagtaaacagtttctttattgggaagaagagtttggtattaaaatttttcctcattttaaagataatcacgatttaccaaaactaactttttttgtggagccttccttggaatttagtccgcacctccctttagccaactgtcttgagcgtttgaaaaaattcgatatttcgcgtgaaagtttgctcgggttagacaataatttttcggccttttctaattatttcaatgagctttttaacttattgtccaggggggagattaaaaagattgtaacagctgtccttgctgtttctaaatcgtgggagaatgagccagaattggaaaagcgcttacattttttgagtgagaaggcaaagttattagggtaccctaagcttacttcttcgtgggcggattatagaatgattattggcggaaaaattaaatcttggcattctaactataccgaacaattaataaaagttagagaggacttaaagaaacatcaaatcgcccttgataaattacaggaagatttaaaaaaagtagtagatagctctttaagagaacaaatagaagctcaacgagaagctttgcttcctttgcttgataccatgttaaaagaaaaagatttttccgatgatttagagctttacagatttatcttgtcagattttaagagtttgttaaatgggtcttatcaaagatatattcaaacagaagaggagagaaaggaggacagagatgttaccaaaaaatataaagatttatatagtaatttgcgcaacatacctagattttttggggaaagtaaaaaggaacaattcaataaatttataaataaatctctcccgaccatagatgttggtttaaaaatacttgaggatattcgtaatgctctagaaactgtaagtgttcgcaaacccccttcaataacagaagagtatgtaacaaagcaacttgagaagttaagtagaaagtacaaaattaacgcctttaattcaaacagatttaaacaaataactgaacaggtgctcagaaaatataataacggagaactaccaaagatctcggaggttttttatagatacccgagagaatctcatgtggctataagaatattacctgttaaaataagcaatccaagaaaggatatatcttatcttctcgacaaatatcaaattagccccgactggaaaaacagtaacccaggagaagttgtagatttgatagagatatataaattgacattgggttggctcttgagttgtaacaaggatttttcgatggatttttcatcgtatgacttgaaactcttcccagaagccgcttccctcataaaaaattttggctcttgcttgagtggttactatttaagcaaaatgatatttaattgcataaccagtgaaataaaggggatgattactttatatactagagacaagtttgttgttagatatgttacacaaatgataggtagcaatcagaaatttcctttgttatgtttggtgggagagaaacagactaaaaacttttctcgcaactggggtgtattgatagaagagaagggagatttgggggaggaaaaaaaccaggaaaaatgtttgatatttaaggataaaacagattttgctaaagctaaagaagtagaaatttttaaaaataatatttggcgtatcagaacctctaagtaccaaatccaatttttgaataggctttttaagaaaaccaaagaatgggatttaatgaatcttgtattgagcgagcctagcttagtattggaggaggaatggggtgtttcgtgggataaagataaacttttacctttactgaagaaagaaaaatcttgcgaagaaagattatattactcacttccccttaacttggtgcctgccacagattataaggagcaatctgcagaaatagagcaaaggaatacatatttgggtttggatgttggagaatttggtgttgcctatgcagtggtaagaatagtaagggacagaatagagcttctgtcctggggattccttaaggacccagctcttcgaaaaataagagagcgtgtacaggatatgaagaaaaagcaggtaatggcagtattttctagctcttccacagctgtcgcgcgagtacgagaaatggctatacactctttaagaaatcaaattcatagcattgctttggcgtataaagcaaagataatttatgagatatctataagcaattttgagacaggtggtaatagaatggctaaaatataccgatctataaaggtttcagatgtttatagggagagtggtgcggataccctagtttcagagatgatctggggcaaaaagaataagcaaatgggaaaccatatatcttcctatgcgacaagttacacttgttgcaattgtgcaagaaccccttttgaacttgttatagataatgacaaggaatatgaaaagggaggcgacgaatttatttttaatgttggcgatgaaaagaaggtaagggggtttttacaaaagagtctgttaggaaaaacaattaaagggaaggaagtgttgaagtctataaaagagtacgcaaggccgcctataagggaagtcttgcttgaaggagaagatgtagagcagttgttgaagaggagaggaaatagctatatttatagatgccctttttgtggatataaaactgatgcggatattcaagcggcgttgaatatagcttgtaggggatatatttcggataacgcaaaggatgctgtgaaggaaggagaaagaaaattagattacattttggaagttagaaaattgtgggagaagaatggagctgttttgagaagcgccaaatttttatagtt

SEQ ID NO: 3 - protein, 1226 aa, Candidatus Vogelbacteria bacterium
MQKVRKTLSEVHKNPYGTKVRNAKTGYSLQIERLSYTGKEGMRSFKIPLENKNKEVFDEFVKKIRNDYISQVGLLNLSDWYEHYQEKQEHYSLADFWLDSLRAGVIFAHKETEIKNLISKIRGDKSIVDKFNASIKKKHADLYALVDIKALYDFLTSDARRGLKTEEEFFNSKRNTLFPKFRKKDNKAVDLWVKKFIGLDNKDKLNFTKKFIGFDPNPQIKYDHTFFFHQDINFDLERITTPKELISTYKKFLGKNKDLYGSDETTEDQLKMVLGFHNNHGAFSKYFNASLEAFRGRDNSLVEQIINNSPYWNSHRKELEKRIIFLQVQSKKIKETELGKPHEYLASFGGKFESWVSNYLRQEEEVKRQLFGYEENKKGQKKFIVGNKQELDKIIRGTDEYEIKAISKETIGLTQKCLKLLEQLKDSVDDYTLSLYRQLIVELRIRLNVEFQETYPELIGKSEKDKEKDAKNKRADKRYPQIFKDIKLIPNFLGETKQMVYKKFIRSADILYEGINFIDQIDKQITQNLLPCFKNDKERIEFTEKQFETLRRKYYLMNSSRFHHVIEGIINNRKLIEMKKRENSELKTFSDSKFVLSKLFLKKGKKYENEVYYTFYINPKARDQRRIKIVLDINGNNSVGILQDLVQKLKPKWDDIIKKNDMGELIDAIEIEKVRLGILIALYCEHKFKIKKELLSLDLFASAYQYLELEDDPEELSGTNLGRFLQSLVCSEIKGAINKISRTEYIERYTVQPMNTEKNYPLLINKEGKATWHIAAKDDLSKKKGGGTVAMNQKIGKNFFGKQDYKTVFMLQDKRFDLLTSKYHLQFLSKTLDTGGGSWWKNKNIDLNLSSYSFIFEQKVKVEWDLTNLDHPIKIKPSENSDDRRLFVSIPFVIKPKQTKRKDLQTRVNYMGIDIGEYGLAWTIINIDLKNKKINKISKQGFIYEPLTHKVRDYVATIKDNQVRGTFGMPDTKLARLRENAITSLRNQVHDIAMRYDAKPVYEFEISNFETGSNKVKVIYDSVKRADIGRGQNNTEADNTEVNLVWGKTSKQFGSQIGAYATSYICSFCGYSPYYEFENSKSGDEEGARDNLYQMKKLSRPSLEDFLQGNPVYKTFRDFDKYKNDQRLQKTGDKDGEWKTHRGNTAIYACQKCRHISDADIQASYWIALKQVVRDFYKDKEMDGDLIQGDNKDKRKVNELNRLIGVHKDVPIINKNLITSLDINLL

SEQ ID NO: 4 - DNA, 2869 bp, Candidatus Vogelbacteria bacterium
atggtattaggttttcataataatcacggcgctttttctaagtatttcaacgcgagcttggaagcttttagggggagagacaactccttggttgaacaaataattaataattctccttactggaatagccatcggaaagaattggaaaagagaatcatttttttgcaagttcagtctaaaaaaataaaagagaccgaactgggaaagcctcacgagtatcttgcgagttttggcgggaagtttgaatcttgggtttcaaactatttacgtcaggaagaagaggtcaaacgtcaactttttggttatgaggagaataaaaaaggccagaaaaaatttatcgtgggcaacaaacaagagctagataaaatcatcagagggacagatgagtatgagattaaagcgatttctaaggaaaccattggacttactcagaaatgtttaaaattacttgaacaactaaaagatagtgtcgatgattatacacttagcctatatcggcaactcatagtcgaattgagaatcagactgaatgttgaattccaagaaacttatccggaattaatcggtaagagtgagaaagataaagaaaaagatgcgaaaaataaacgggcagacaagcgttacccgcaaatttttaaggatataaaattaatccccaattttctcggtgaaacgaaacaaatggtatataagaaatttattcgttccgctgacatcctttatgaaggaataaattttatcgaccagatcgataaacagattactcaaaatttgttgccttgttttaagaacgacaaggaacggattgaatttaccgaaaaacaatttgaaactttacggcgaaaatactatctgatgaatagttcccgttttcaccatgttattgaaggaataatcaataataggaaacttattgaaatgaaaaagagagaaaatagcgagttgaaaactttctccgatagtaagtttgttttatctaagctttttcttaaaaaaggcaaaaaatatgaaaatgaggtctattatactttttatataaatccgaaagctcgtgaccagcgacggataaaaattgttcttgatataaatgggaacaattcagtcggaattttacaagatcttgtccaaaagttgaaaccaaaatgggacgacatcataaagaaaaatgatatgggagaattaatcgatgcaatcgagattgagaaagtccggctcggcatcttgatagcgttatactgtgagcataaattcaaaattaaaaaagaactcttgtcattagatttgtttgccagtgcctatcaatatctagaattggaagatgaccctgaagaactttctgggacaaacctaggtcggtttttacaatccttggtctgctccgaaattaaaggtgcgattaataaaataagcaggacagaatatatagagcggtatactgtccagccgatgaatacggagaaaaactatcctttactcatcaataaggagggaaaagccacttggcatattgctgctaaggatgacttgtccaagaagaagggtgggggcactgtcgctatgaatcaaaaaatcggcaagaatttttttgggaaacaagattataaaactgtgtttatgcttcaggataagcggtttgatctactaacctcaaagtatcacttgcagtttttatctaaaactcttgatactggtggagggtcttggtggaaaaacaaaaatattgatttaaatttaagctcttattctttcattttcgaacaaaaagtaaaagtcgaatgggatttaaccaatcttgaccatcctataaagattaagcctagcgagaacagtgatgatagaaggcttttcgtatccattccttttgttattaaaccgaaacagacaaaaagaaaggatttgcaaactcgagtcaattatatggggattgatatcggagaatatggtttggcttggacaattattaatattgatttaaagaataaaaaaataaataagatttcaaaacaaggtttcatctatgagccgttgacacataaagtgcgcgattatgttgctaccattaaagataatcaggttagaggaacttttggcatgcctgatacgaaactagccagattgcgagaaaatgccattaccagcttgcgcaatcaagtgcatgatattgctatgcgctatgacgccaaaccggtatatgaatttgaaatttccaattttgaaacggggtctaataaagtgaaagtaatttatgattcggttaagcgagctgatatcggccgaggccagaataataccgaagcagacaatactgaggttaatcttgtctgggggaagacaagcaaacaatttggcagtcaaatcggcgcttatgcgacaagttacatctgttcattttgtggttattctccatattatgaatttgaaaattctaagtcgggagatgaagaaggggctagagataatctatatcagatgaagaaattgagtcgcccctctcttgaagatttcctccaaggaaatccggtttataagacatttagggattttgataagtataaaaacgatcaacggttgcaaaagacgggtgataaagatggtgaatggaaaacacacagagggaatactgcaatatacgcctgtcaaaagtgtagacatatctctgatgcggatatccaagcatcatattggattgctttgaagcaagttgtaagagatttttataaagacaaagagatggatggtgatttgattcaaggagataataaagacaagagaaaagtaaacgagcttaatagacttattggagtacataaagatgtgcctataataaataaaaatttaataacatcactcgacataaacttactataga

SEQ ID NO: 5 - protein, 1200 aa, Candidatus Vogelbacteria bacterium
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLL

SEQ ID NO: 6 - DNA, 3604 bp, Candidatus Vogelbacteria bacterium
atgaaagctaaaaaaagtttttataatcaaaagcggaagttcggtaaaagaggttatcgtcttcacgatgaacgtatcgcgtattcaggagggattggatcgatgcgatctattaaatatgaattgaaggattcgtatggaattgctgggcttcgtaatcgaatcgctgacgcaactatttctgataataagtggctgtacgggaatataaatctaaatgattatttagagtggcgatcttcaaagactgacaaacagattgaagacggagaccgagaatcatcactcctgggtttttggctggaagcgttacgactgggattcgtgttttcaaaacaatctcatgctccgaatgattttaacgagaccgctctacaagatttgtttgaaactcttgatgatgatttgaaacatgttcttgataggaaaaaatggtgtgactttatcaagataggaacacctaagacaaatgaccaaggtcgtttaaaaaaacaaatcaagaatttgttaaaaggaaacaagagagaggaaattgaaaaaactctcaatgaatcagacgatgaattgaaagagaaaataaacagaattgccgatgtttttgcaaaaaataagtctgataaatacacaattttcaaattagataaacccaatacggaaaaataccccagaatcaacgatgttcaggtggcgtttttttgtcatcccgattttgaggaaattacagaacgagatagaacaaagactctagatctgatcattaatcggtttaataagagatatgaaattaccgaaaataaaaaagatgacaaaacttcaaacaggatggccttgtattccttgaaccagggctatattcctcgcgtcctgaatgatttattcttgtttgtcaaagacaatgaggatgattttagtcagtttttatctgatttggagaatttcttctctttttccaacgaacaaattaaaataataaaggaaaggttaaaaaaacttaaaaaatatgctgaaccaattcccggaaagccgcaacttgctgataaatgggacgattatgcttctgattttggcggtaaattggaaagctggtactccaatcgaatagagaaattaaagaagattccggaaagcgtttccgatctgcggaataatttggaaaagatacgcaatgttttaaaaaaacaaaataatgcatctaaaatcctggagttatctcaaaagatcattgaatacatcagagattatggagtttcttttgaaaagccggagataattaagttcagctggataaataagacgaaggatggtcagaaaaaagttttctatgttgcgaaaatggcggatagagaattcatagaaaagcttgatttatggatggctgatttacgcagtcaattaaatgaatacaatcaagataataaagtttctttcaaaaagaaaggtaaaaaaatagaagagctcggtgtcttggattttgctcttaataaagcgaaaaaaaataaaagtacaaaaaatgaaaatggctggcaacaaaaattgtcagaatctattcaatctgccccgttattttttggcgaagggaatcgtgtacgaaatgaagaagtttataatttgaaggaccttctgttttcagaaatcaagaatgttgaaaatattttaatgagctcggaagcggaagacttaaaaaatataaaaattgaatataaagaagatggcgcgaaaaaagggaactatgtcttgaatgtcttggctagattttacgcgagattcaatgaggatggctatggtggttggaacaaagtaaaaaccgttttggaaaatattgcccgagaggcggggactgatttttcaaaatatggaaataataacaatagaaatgccggcagattttatctaaacggccgcgaacgacaagtttttactctaatcaagtttgaaaaaagtatcacggtggaaaaaatacttgaattggtaaaattacctagcctacttgatgaagcgtatagagatttagtcaacgaaaataaaaatcataaattacgcgacgtaattcaattgagcaagacaattatggctctggttttatctcattctgataaagaaaaacaaattggaggaaattatatccatagtaaattgagcggatacaatgcgcttatttcaaagcgagattttatctcgcggtatagcgtgcaaacgaccaacggaactcaatgtaaattagccataggaaaaggcaaaagcaaaaaaggtaatgaaattgacaggtatttctacgcttttcaattttttaagaatgacgacagcaaaattaatttaaaggtaatcaaaaataattcgcataaaaacatcgatttcaacgacaatgaaaataaaattaacgcattgcaagtgtattcatcaaactatcagattcaattcttagactggttttttgaaaaacatcaagggaagaaaacatcgctcgaggtcggcggatcttttaccatcgccgaaaagagtttgacaatagactggtcggggagtaatccgagagtcggttttaaaagaagcgacacggaagaaaagagggtttttgtctcgcaaccatttacattaataccagacgatgaagacaaagagcgtcgtaaagaaagaatgataaagacgaaaaaccgttttatcggtatcgatatcggtgaatatggtctggcttggagtctaatcgaagtggacaatggagataaaaataatagaggaattagacaacttgagagcggttttattacagacaatcagcagcaagtcttaaagaaaaacgtaaaatcctggaggcaaaaccaaattcgtcaaacgtttacttcaccagacacaaaaattgctcgtcttcgtgaaagtttgatcggaagttacaaaaatcaactggaaagtctgatggttgctaaaaaagcaaatcttagttttgaatacgaagtttccgggtttgaagttgggggaaagagggttgcaaaaatatacgatagtataaagcgtgggtcggtgcgtaaaaaggataataactcacaaaatgatcaaagttggggtaaaaagggaattaatgagtggtcattcgagacgacggctgccggaacatcgcaattttgtactcattgcaagcggtggagcagtttagcgatagtagatattgaagaatatgaattaaaagattacaacgataatttatttaaggtaaaaattaatgatggtgaagttcgtctccttggtaagaaaggttggagatccggcgaaaagatcaaagggaaagaattatttggtcccgtcaaagacgcaatgcgcccaaatgttgacggactagggatgaaaattgtaaaaagaaaatatctaaaacttgatctccgcgattgggtttcaagatatgggaatatggctattttcatctgtccttatgtcgattgccaccatatctctcatgcggataaacaagctgcttttaatattgccgtgcgagggtatttgaaaagcgttaatcctgacagagcaataaaacacggagataaaggtttgtctagggactttttgtgccaagaagagggtaagcttaattttgaacaaatagggttattatgaa

SEQ ID NO: 7 - protein, 1210 aa, Unknown
  Description of Unknown Candidatus Parcubacteria sequence
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 8 - DNA, 3636 bp, Unknown
  Description of Unknown Candidatus Parcubacteria sequence
atgagtaagcgacatcctagaattagcggcgtaaaagggtaccgtttgcatgcgcaacggctggaatataccggcaaaagtggggcaatgcgaacgattaaatatcctctttattcatctccgagcggtggaagaacggttccgcgcgagatagtttcagcaatcaatgatgattatgtagggctgtacggtttgagtaattttgacgatctgtataatgcggaaaagcgcaacgaagaaaaggtctactcggttttagatttttggtacgactgcgtccaatacggcgcggttttttcgtatacagcgccgggtcttttgaaaaatgttgccgaagttcgcgggggaagctacgaacttacaaaaacgcttaaagggagccatttatatgatgaattgcaaattgataaagtaattaaatttttgaataaaaaagaaatttcgcgagcaaacggatcgcttgataaactgaagaaagacatcattgattgcttcaaagcagaatatcgggaacgacataaagatcaatgcaataaactggctgatgatattaaaaatgcaaaaaaagacgcgggagcttctttaggggagcgtcaaaaaaaattatttcgcgatttttttggaatttcagagcagtctgaaaatgataaaccgtcttttactaatccgctaaacttaacctgctgtttattgccttttgacacagtgaataacaacagaaaccgcggcgaagttttgtttaacaagctcaaggaatatgctcaaaaattggataaaaacgaagggtcgcttgaaatgtgggaatatattggcatcgggaacagcggcactgccttttctaattttttaggagaagggtttttgggcagattgcgcgagaataaaattacagagctgaaaaaagccatgatggatattacagatgcatggcgtgggcaggaacaggaagaagagttagaaaaacgtctgcggatacttgccgcgcttaccataaaattgcgcgagccgaaatttgacaaccactggggagggtatcgcagtgatataaacggcaaattatctagctggcttcagaattacataaatcaaacagtcaaaatcaaagaggacttaaagggacacaaaaaggacctgaaaaaagcgaaagagatgataaataggtttggggaaagcgacacaaaggaagaggcggttgtttcatctttgcttgaaagcattgaaaaaattgttcctgatgatagcgctgatgacgagaaacccgatattccagctattgctatctatcgccgctttctttcggatggacgattaacattgaatcgctttgtccaaagagaagatgtgcaagaggcgctgataaaagaaagattggaagcggagaaaaagaaaaaaccgaaaaagcgaaaaaagaaaagtgacgctgaagatgaaaaagaaacaattgacttcaaggagttatttcctcatcttgccaaaccattaaaattggtgccaaacttttacggcgacagtaagcgtgagctgtacaagaaatataagaacgccgctatttatacagatgctctgtggaaagcagtggaaaaaatatacaaaagcgcgttctcgtcgtctctaaaaaattcattttttgatacagattttgataaagatttttttattaagcggcttcagaaaattttttcggtttatcgtcggtttaatacagacaaatggaaaccgattgtgaaaaactctttcgcgccctattgcgacatcgtctcacttgcggagaatgaagttttgtataaaccgaaacagtcgcgcagtagaaaatctgccgcgattgataaaaacagagtgcgtctcccttccactgaaaatatcgcaaaagctggcattgccctcgcgcgggagctttcagtcgcaggatttgactggaaagatttgttaaaaaaagaggagcatgaagaatacattgatctcatagaattgcacaaaaccgcgcttgcgcttcttcttgccgtaacagaaacacagcttgacataagcgcgttggattttgtagaaaatgggacggtcaaggattttatgaaaacgcgggacggcaatctggttttggaagggcgtttccttgaaatgttctcgcagtcaattgtgttttcagaattgcgcgggcttgcgggtttaatgagccgcaaggaatttatcactcgctccgcgattcaaactatgaacggcaaacaggcggagcttctctacattccgcatgaattccaatcggcaaaaattacaacgccaaaggaaatgagcagggcgtttcttgaccttgcgcccgcggaatttgctacatcgcttgagccagaatcgctttcggagaagtcattattgaaattgaagcagatgcggtactatccgcattattttggatatgagcttacgcgaacaggacaggggattgatggtggagtcgcggaaaatgcgttacgacttgagaagtcgccagtaaaaaaacgagagataaaatgcaaacagtataaaactttgggacgcggacaaaataaaatagtgttatatgtccgcagttcttattatcagacgcaatttttggaatggtttttgcatcggccgaaaaacgttcaaaccgatgttgcggttagcggttcgtttcttatcgacgaaaagaaagtaaaaactcgctggaattatgacgcgcttacagtcgcgcttgaaccagtttccggaagcgagcgggtctttgtctcacagccgtttactatttttccggaaaaaagcgcagaggaagaaggacagaggtatcttggcatagacatcggcgaatacggcattgcgtatactgcgcttgagataactggcgacagtgcaaagattcttgatcaaaattttatttcagacccccagcttaaaactctgcgcgaggaggtcaaaggattaaaacttgaccaaaggcgcgggacatttgccatgccaagcacgaaaatcgcccgcatccgcgaaagccttgtgcatagtttgcggaaccgcatacatcatcttgcgttaaagcacaaagcaaagattgtgtatgaattggaagtgtcgcgttttgaagagggaaagcaaaaaattaagaaagtctacgctacgttaaaaaaagcggatgtgtattcagaaattgacgcggataaaaatttacaaacgacagtatggggaaaattggccgttgcaagcgaaatcagcgcaagctatacaagccagttttgtggtgcgtgtaaaaaattgtggcgggcggaaatgcaggttgacgaaacaattacaacccaagaactaatcggcacagttagagtcataaaagggggcactcttattgacgcgataaaggattttatgcgcccgccgatttttgacgaaaatgacactccatttccaaaatatagagacttttgcgacaagcatcacatttccaaaaaaatgcgtggaaacagctgtttgttcatttgtccattctgccgcgcaaacgcggatgctgatattcaagcaagccaaacaattgcgcttttaaggtatgttaaggaagagaaaaaggtagaggactactttgaacgatttagaaagctaaaaaacattaaagtgctcggacagatgaagaaaatatgatag

SEQ ID NO: 9 - protein, 1192 aa, Unknown
  Description of Unknown Candidatus Komeilibacteria sequence
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 10 - DNA, 4560 bp, Unknown
  Description of Unknown Candidatus Komeilibacteria sequence
accaaccacctattgcgtctttttcgctcattttagcaaaagtggctgtctagacatacaggtggaaaggtgagagtaaagacatggcctgaatagcgtcctcgtcctcgtctagacatacaggtggaaaggtgagagtaaagaccggagcactcatcctctcactctattttgtctagacatacaggtggaaaggtgagagtaaagacaaaccgtgccacactaaaccgatgagtctagacatacaggtggaaaggtgagagtaaagactcaagtaactacctgttctttcacaagtctagacatacaggtggaaaggtgagagtaaagactcaagtaactacctgttctttcacaagtctagacctgcaggtggtaaggtgagagtaaagactcaagtaactacctgttctttcacaagtctagacctgcaggtggtaaggtgagagtaaagacttttatcctcctctctatgcttctgagtctagacatttaggtggaaaggtgagagtaaagacttgtggagatccatgaacttcggcagtctagacctgcaggtggaaaggtgagagtaaagacgtccttcacacgatcttcctctgttagtctaggcctgcaggtggaaaggtgagagtaaagacgcataagcgtaattgaagctctctccggtccagaccttgtcgcgcttgtgttgcgacaaaggcggagtccgcaataagttctttttacaatgttttttccataaaaccgatacaatcaagtatcggttttgctttttttatgaaaatatgttatgctatgtgctcaaataaaaatatcaataaaatagcgtttttttgataatttatcgctaaaattatacataatcacgcaacattgccattctcacacaggagaaaagtcatggcagaaagcaagcagatgcaatgccgcaagtgcggcgcaagcatgaagtatgaagtaattggattgggcaagaagtcatgcagatatatgtgcccagattgcggcaatcacaccagcgcgcgcaagattcagaacaagaaaaagcgcgacaaaaagtatggatccgcaagcaaagcgcagagccagaggatagctgtggctggcgcgctttatccagacaaaaaagtgcagaccataaagacctacaaatacccagcggatctgaatggcgaagttcatgacagaggcgtcgcagagaagattgagcaggcgattcaggaagatgagatcggcctgcttggcccgtccagcgaatacgcttgctggattgcttcacaaaaacaaagcgagccgtattcagttgtagatttttggtttgacgcggtgtgcgcaggcggagtattcgcgtattctggcgcgcgcctgctttccacagtcctccagttgagtggcgaggaaagcgttttgcgcgctgctttagcatctagcccgtttgtagatgacattaatttggcgcaagcggaaaagttcctagccgttagccggcgcacaggccaagataagctaggcaagcgcattggagaatgtttcgcggaaggccggcttgaagcgcttggcatcaaagatcgcatgcgcgaattcgtgcaagcgattgatgtggcccaaaccgcgggccagcggttcgcggccaagctaaagatattcggcatcagtcagatgcctgaagccaagcaatggaacaatgattccgggctcactgtatgtattttgccggattattatgtcccggaagaaaaccgcgcggaccagctggttgttttgcttcggcgcttacgcgagatcgcgtattgcatgggaattgaggatgaagcaggatttgagcatctaggcattgaccctggcgctctttccaatttttccaatggcaatccaaagcgaggatttctcggccgcctgctcaataatgacattatagcgctggcaaacaacatgtcagccatgacgccgtattgggaaggcagaaaaggcgagttgattgagcgccttgcatggcttaaacatcgcgctgaaggattgtatttgaaagagccacatttcggcaactcctgggcagaccaccgcagcaggattttcagtcgcattgcgggctggctttccggatgcgcgggcaagctcaagattgccaaggatcagatttcaggcgtgcgtacggatttgtttctgctcaagcgccttctggatgcggtaccgcaaagcgcgccgtcgccggactttattgcttccatcagcgcgctggatcggtttttggaagcggcagaaagcagccaggatccggcagaacaggtacgcgctttgtacgcgtttcatctgaacgcgcctgcggtccgatccatcgccaacaaggcggtacagaggtctgattcccaggagtggcttatcaaggaactggatgctgtagatcaccttgaattcaacaaagcatttccgtttttttcggatacaggaaagaaaaagaagaaaggagcgaatagcaacggagcgccttctgaagaagaatacacggaaacagaatccattcaacaaccagaagatgcagagcaggaagtgaatggtcaagaaggaaatggcgcttcaaagaaccagaaaaagtttcagcgcattcctcgatttttcggggaagggtcaaggagtgagtatcgaattttaacagaagcgccgcaatattttgacatgttctgcaataatatgcgcgcgatctttatgcagctagagagtcagccgcgcaaggcgcctcgtgatttcaaatgctttctgcagaatcgtttgcagaagctttacaagcaaacctttctcaatgctcgcagtaataaatgccgcgcgcttctggaatccgtccttatttcatggggagaattttatacttatggcgcgaatgaaaagaagtttcgtctgcgccatgaagcgagcgagcgcagctcggatccggactatgtggttcagcaggcattggaaatcgcgcgccggcttttcttgttcggatttgagtggcgcgattgctctgctggagagcgcgtggatttggttgaaatccacaaaaaagcaatctcatttttgcttgcaatcactcaggccgaggtttcagttggttcctataactggcttgggaatagcaccgtgagccggtatctttcggttgctggcacagacacattgtacggcactcaactggaggagtttttgaacgccacagtgctttcacagatgcgtgggctggcgattcggctttcatctcaggagttaaaagacggatttgatgttcagttggagagttcgtgccaggacaatctccagcatctgctggtgtatcgcgcttcgcgcgacttggctgcgtgcaaacgcgctacatgcccggctgaattggatccgaaaattcttgttctgccggctggtgcgtttatcgcgagcgtaatgaaaatgattgagcgtggcgatgaaccattagcaggcgcgtatttgcgtcatcggccgcattcattcggctggcagatacgggttcgtggagtggcggaagtaggcatggatcagggcacagcgctagcattccagaagccgactgaatcagagccgtttaaaataaagccgttttccgctcaatacggcccagtactttggcttaattcttcatcctatagccagagccagtatctggatggatttttaagccagccaaagaattggtctatgcgggtgctacctcaagccggatcagtgcgcgtggaacagcgcgttgctctgatatggaatttgcaggcaggcaagatgcggctggagcgctctggagcgcgcgcgtttttcatgccagtgccattcagcttcaggccgtctggttcaggagatgaagcagtattggcgccgaatcggtacttgggactttttccgcattccggaggaatagaatacgcggtggtggatgtattagattccgcgggtttcaaaattcttgagcgcggtacgattgcggtaaatggcttttcccagaagcgcggcgaacgccaagaggaggcacacagagaaaaacagagacgcggaatttctgatataggccgcaagaagccggtgcaagctgaagttgacgcagccaatgaattgcaccgcaaatacaccgatgttgccactcgtttagggtgcagaattgtggttcagtgggcgccccagccaaagccgggcacagcgccgaccgcgcaaacagtatacgcgcgcgcagtgcggaccgaagcgccgcgatctggaaatcaagaggatcatgctcgtatgaaatcctcttggggatatacctggagcacctattgggagaagcgcaaaccagaggatattttgggcatctcaacccaagtatactggaccggcggtataggcgagtcatgtcccgcagtcgcggttgcgcttttggggcacattagggcaacatccactcaaactgaatgggaaaaagaggaggttgtattcggtcgactgaagaagttctttccaagctagacgatctttttaaaaactgggctgctggctatcgtatggtcagtagctcttatttttttacttgatatatggtattat

SEQ ID NO: 11 - protein, 1287 aa, Candidatus Kerfeldbacteria bacterium
MKRILNSLKVAALRLLFRGKGSELVKTVKYPLVSPVQGAVEELAEAIRHDNLHLFGQKEIVDLMEKDEGTQVYSVVDFWLDTLRLGMFFSPSANALKITLGKFNSDQVSPFRKVLEQSPFFLAGRLKVEPAERILSVEIRKIGKRENRVENYAADVETCFIGQLSSDEKQSIQKLANDIWDSKDHEEQRMLKADFFAIPLIKDPKAVTEEDPENETAGKQKPLELCVCLVPELYTRGFGSIADFLVQRLTLLRDKMSTDTAEDCLEYVGIEEEKGNGMNSLLGTFLKNLQGDGFEQIFQFMLGSYVGWQGKEDVLRERLDLLAEKVKRLPKPKFAGEWSGHRMFLHGQLKSWSSNFFRLFNETRELLESIKSDIQHATMLISYVEEKGGYHPQLLSQYRKLMEQLPALRTKVLDPEIEMTHMSEAVRSYIMIHKSVAGFLPDLLESLDRDKDREFLLSIFPRIPKIDKKTKEIVAWELPGEPEEGYLFTANNLFRNFLENPKHVPRFMAERIPEDWTRLRSAPVWFDGMVKQWQKVVNQLVESPGALYQFNESFLRQRLQAMLTVYKRDLQTEKFLKLLADVCRPLVDFFGLGGNDIIFKSCQDPRKQWQTVIPLSVPADVYTACEGLAIRLRETLGFEWKNLKGHEREDFLRLHQLLGNLLFWIRDAKLVVKLEDWMNNPCVQEYVEARKAIDLPLEIFGFEVPIFLNGYLFSELRQLELLLRRKSVMTSYSVKTTGSPNRLFQLVYLPLNPSDPEKKNSNNFQERLDTPTGLSRRFLDLTLDAFAGKLLTDPVTQELKTMAGFYDHLFGFKLPCKLAAMSNHPGSSSKMVVLAKPKKGVASNIGFEPIPDPAHPVFRVRSSWPELKYLEGLLYLPEDTPLTIELAETSVSCQSVSSVAFDLKNLTTILGRVGEFRVTADQPFKLTPIIPEKEESFIGKTYLGLDAGERSGVGFAIVTVDGDGYEVQRLGVHEDTQLMALQQVASKSLKEPVFQPLRKGTFRQQERIRKSLRGCYWNFYHALMIKYRAKVVHEESVGSSGLVGQWLRAFQKDLKKADVLPKKGGKNGVDKKKRESSAQDTLWGGAFSKKEEQQIAFEVQAAGSSQFCLKCGWWFQLGMREVNRVQESGVVLDWNRSIVTFLIESSGEKVYGFSPQQLEKGFRPDIETFKKMVRDFMRPPMFDRKGRPAAAYERFVLGRRHRRYRFDKVFEERFGRSALFICPRVGCGNFDHSSEQSAVVLALIGYIADKEGMSGKKLVYVRLAELMAEWKLKKLERSRVEEQSSAQ

SEQ ID NO: 12 - DNA, 3864 bp, Candidatus Kerfeldbacteria bacterium
atgaagagaattctgaacagtctgaaagttgctgccttgagacttctgtttcgaggcaaaggttctgaattagtgaagacagtcaaatatccattggtttccccggttcaaggcgcggttgaagaacttgctgaagcaattcggcacgacaacctgcacctttttgggcagaaggaaatagtggatcttatggagaaagacgaaggaacccaggtgtattcggttgtggatttttggttggataccctgcgtttagggatgtttttctcaccatcagcgaatgcgttgaaaatcacgctgggaaaattcaattctgatcaggtttcaccttttcgtaaggttttggagcagtcacctttttttcttgcgggtcgcttgaaggttgaacctgcggaaaggatactttctgttgaaatcagaaagattggtaaaagagaaaacagagttgagaactatgccgccgatgtggagacatgcttcattggtcagctttcttcagatgagaaacagagtatccagaagctggcaaatgatatctgggatagcaaggatcatgaggaacagagaatgttgaaggcggatttttttgctatacctcttataaaagaccccaaagctgtcacagaagaagatcctgaaaatgaaacggcgggaaaacagaaaccgcttgaattatgtgtttgtcttgttcctgagttgtatacccgaggtttcggctccattgctgattttctggttcagcgacttaccttgctgcgtgacaaaatgagtaccgacacggcggaagattgcctcgagtatgttggcattgaggaagaaaaaggcaatggaatgaattccttgctcggcacttttttgaagaacctgcagggtgatggttttgaacagatttttcagtttatgcttgggtcttatgttggctggcaggggaaggaagatgtactgcgcgaacgattggatttgctggccgaaaaagtcaaaagattaccaaagccaaaatttgccggagaatggagtggtcatcgtatgtttctccatggtcagctgaaaagctggtcgtcgaatttcttccgtctttttaatgagacgcgggaacttctggaaagtatcaagagtgatattcaacatgccaccatgctcattagctatgtggaagagaaaggaggctatcatccacagctgttgagtcagtatcggaagttaatggaacaattaccggcgttgcggactaaggttttggatcctgagattgagatgacgcatatgtccgaggctgttcgaagttacattatgatacacaagtctgtagcgggatttctgccggatttactcgagtctttggatcgagataaggatagggaatttttgctttccatctttcctcgtattccaaagatagataagaagacgaaagagatcgttgcatgggagctaccgggcgagccagaggaaggctatttgttcacagcaaacaaccttttccggaattttcttgagaatccgaaacatgtgccacgatttatggcagagaggattcccgaggattggacgcgtttgcgctcggcccctgtgtggtttgatgggatggtgaagcaatggcagaaggtggtgaatcagttggttgaatctccaggcgccctttatcagttcaatgaaagttttttgcgtcaaagactgcaagcaatgcttacggtctataagcgggatctccagactgagaagtttctgaagctgctggctgatgtctgtcgtccactcgttgattttttcggacttggaggaaatgatattatcttcaagtcatgtcaggatccaagaaagcaatggcagactgttattccactcagtgtcccagcggatgtttatacagcatgtgaaggcttggctattcgtctccgcgaaactcttggattcgaatggaaaaatctgaaaggacacgagcgggaagattttttacggctgcatcagttgctgggaaatctgctgttctggatcagggatgcgaaacttgtcgtgaagctggaagactggatgaacaatccttgtgttcaggagtatgtggaagcacgaaaagccattgatcttcccttggagattttcggatttgaggtgccgatttttctcaatggctatctcttttcggaactgcgccagctggaattgttgctgaggcgtaagtcggtgatgacgtcttacagcgtcaaaacgacaggctcgccaaataggctcttccagttggtttacctacctctaaacccttcagatccggaaaagaaaaattccaacaactttcaggagcgcctcgatacacctaccggtttgtcgcgtcgttttctggatcttacgctggatgcatttgctggcaaactcttgacggatccggtaactcaggaactgaagacgatggccggtttttacgatcatctctttggcttcaagttgccgtgtaaactggcggcgatgagtaaccatccaggatcctcttccaaaatggtggttctggcaaaaccaaagaagggtgttgctagtaacatcggctttgaacctattcccgatcctgctcatcctgtgttccgggtgagaagttcctggccggagttgaagtacctggaggggttgttgtatcttcccgaagatacaccactgaccattgaactggcggaaacgtcggtcagttgtcagtctgtgagttcagtcgctttcgatttgaagaatctgacgactatcttgggtcgtgttggtgaattcagggtgacggcagatcaacctttcaagctgacgcccattattcctgagaaagaggaatccttcatcgggaagacctacctcggtcttgatgctggagagcgatctggcgttggtttcgcgattgtgacggttgacggcgatgggtatgaggtgcagaggttgggtgtgcatgaagatactcagcttatggcgcttcagcaagtcgccagcaagtctcttaaggagccggttttccagccactccgtaagggcacatttcgtcagcaggagcgcattcgcaaaagcctccgcggttgctactggaatttctatcatgcattgatgatcaagtaccgagctaaagttgtgcatgaggaatcggtgggttcatccggtctggtggggcagtggctgcgtgcatttcagaaggatctcaaaaaggctgatgttctgcccaagaagggtggaaaaaatggtgtagacaaaaaaaagagagaaagcagcgctcaggataccttatggggaggagctttctcgaagaaggaagagcagcagatagcctttgaggttcaggcagctggatcaagccagttttgtctgaagtgtggttggtggtttcagttggggatgcgggaagtaaatcgtgtgcaggagagtggcgtggtgctggactggaaccggtccattgtaaccttcctcatcgaatcctcaggagaaaaggtatatggtttcagtcctcagcaactggaaaaaggctttcgtcctgacatcgaaacgttcaaaaaaatggtaagggattttatgagaccccccatgtttgatcgcaaaggtcggccggccgcggcgtatgaaagattcgtactgggacgtcgtcaccgtcgttatcgctttgataaagtttttgaagagagatttggtcgcagtgctcttttcatctgcccgcgggtcgggtgtgggaatttcgatcactccagtgagcagtcagccgttgtccttgcccttattggttacattgctgataaggaagggatgagtggtaagaagcttgtttatgtgaggctggctgaacttatggctgagtggaagctgaagaaactggagagatcaagggtggaagaacagagctcggcacaataa

SEQ ID NO: 13 - protein, 978 aa, Planctomycetes bacterium
MQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 14 - DNA, 5495 bp, Planctomycetes bacterium
atgcttcttatttatcggagatatcttcaaacaccatcaacatggcaatggtgaaccattaatattctttgatgcttcttatttatcggagatatcttcaaacattgcccattttacaggcatatcttctggctctttgatgcttcttatttatcggagatatcttcaaacgtaatgtattgagaaagacatcaagattagataactttgatgcttcttatttatcggagatatcttcaaacacagaaacctgcaaagattgtatatatataagctttgatgcttcttatttatcggagatatcttcaaacgatacgtattttagcccgtctatttggggattaactttgatgcttcttatttatcggagatatcttcaaaccccgcatatccagatttttcaatgacttctggaaattgtattttcaatattttacaagttgcggaggatacctttaataatttagcagagttacgcactgtaaacctgttcttctcacaaaaagctttaacatcagattttcaaagaacttcttatgtaatttataagaatctaaaaaaacagctctgggtttgcatccagaactctccgataaataagcgctttacccatacgacatagtcgctggtgatggctctcaaagtaatgagataaaagcgccagtaataatttactattcacaaatcctttcgtcaagcttaaaatcaatcaaagaccatatccccttcattccaaatagcagcgcttccgtacctttctatccgttcatatatctcctctgagagaggataaattaccagacttatagagccatccataaatcctttttctttaaggttgagctttagatcagcccaccttgcttttgaaaggttaaactcaaagacagaatattgaatccgaacaccataggcttccagaagtttaactaaccgtgccctgaccttatcatcttcaatatcataacaaatgagatgtcgcattttaaagctctataggcttataacattccctatcatcttgaatatgctggctaaacaacctaacctgccgctcaactgcgtgctgatacgttattgattggataagtaaattggttttctgctcatctaccttaaagaattgatgccattttttgattacttttggataggcatccttattcagccaaacacctttttggtcagtttctttcctgaaatcgtctgtatccacttcccttctatttatcaaattgatcacaaaacggtcagccaacggccgccactcctccagaagatcgcatattaaagagggacgaccataatagacgtcatgcaagtaaccaaaggccgggtcaaaaccgacgagtaatgcagtcgaatgtatttcgttgaacaggagggtgtagataaggctcatcatggcgttgatttcatcctcaggaggtctcttggtacggcgcacaaaaacaaagcttggatgctttaagatagccgaaaaattgccataatactgccttgttgttgcgccttctattccacgcaaggtctctaaatcagtgacggcgttgatttcggtacactcgattctcaaaccaagtctatatttatcaagtaatgattgctggtttttgatcttaccggcaacgatactttttgcaatttcaagttttttgtggggatcaaaatgcttatgaatttgcgcccgacgaataaacagatttttgacgggttcaaattgaaggctcccttgatattcccatctgccgctaaagaaatgtatcggtatagattattctctgcaaaggctaataacacggctatcgagggtaacccggccaactaccacgatatcttttaccttcattgcgggaatcttctgccccttctcttcattgtccttttttatgagaaatgcccgaccacgacaatccaaaatgaattcatcacccgtgagatagagggttatcctgtcggttatagcggtcatcagtaagccttttatttttctaaccaagtattgaaggaagacacgattcactatactggcactgcggacacctatggtcatcaaccttgggaaacctgcttatatcaaaggacaagaagcagtctcgcagatttgtaacaacttctacacaacgcactttcagggttttatctataacaatttctttccgtctccgtgtttcacagaaaaatatttcaccaactggtatattgacattatacatctcttcaaggcaaattgcctgtaacccaatctgaacgtggaagttctcaaaatcccttaccttccctgtctttgtttcgataggaatcggtatcccatccctccactcgataaggtctgcccggcctgccaaaccgagcttattgctgtaaagatacacgcctgttacctgcttacaatcagggcagcttctctgcgatgatttatccaccgccctgtgcgcgtgtatggcctctgtaaagtggatgctcttagccatattacgccgttctccaacaaaggcataccatgcattgcgcggacaatagattgactccattaccgtgctgatgtgcaatatcagacggctggtttccatacttctttgagcttctttctgtaaaaggattgccatgtttcaacaaatgcccttttgtcagtatttccggtcgttttattggtttgatacttcttatattcttgagaacggagaaagagccacgaccttgcaatattcagtgctgcttgttcgtctgcatgggtttcaaaaccacagttcaggcaaacaaacttttcctgcaccggcctgtgactaaatctcttttttagcagagataaagcttcaccactgcggccttttgtccaactagaaatatcattatttaccgactcttccgaaagtctatccagctctacagagaggtcttttaccacattctgccttttataccggttatagtatgttatctgtccttcaacttttaactcttttccattgattgtagtcatccatccagtagccgtcttcttgagcttttcgagcaccctgtcataatctgcacttgtgattgtaaaaccacaattagaacatgtctttgaggtatactgtgccagagtctttgaaagataggtttttgatggcagaccttcataggcaagctttgcagtcagccagtcttccatcctcgtgtactgcctttccgccataaaagtcctcttgccttgtctaccaaaaccgcgggaaagattttcaaaaatgagcattgcatcttgagtaacagcataatataagaggtcacgagctgtatttcttaccatatcgtccgccagattcttcgcctttgatgcatattttctcgaatatccgcctgcccgcctttgttcaacttctttagcagcctgaatagtccgttgtttttccttataactttctcctattcgcaaaatatgcgttggattgcccaatgaatctttgaatcttgacaaggggcatccttccgggtctgttaatgctatgactgccgggatattttctccccggtctattcctatcagattcatcggttttatattcgatgagtcaagcacctctcttctttcaaatgtcagggcaacaaaaagtgctggttcatcctgtctcgtccttctgttatagagcgttttttcaataaccctgccattggcgagtttcaatgaacccgtctcaaggctcaataggtcgttccagataaactccctcccctgcctttttccaaaggccaaaggcagaattatcaaattcgggtcatcaaaattgaagttgacctccataggcacaatctcaccgctttttttattaattactgtataaaacctatttgcttcaaaagcttctggcttgatttttttgaagcgtagcttaccacctttgaagtaatttattattaaataaagatttaacttctttacgccgtctttctgccatataaatgcacaattatactgtttagaaaatccgcttatatctaaaatgctgttctctgcttctatagcaaatggttttcctctcaaatctccataccacttttgaagctttaactcacacctgcaaaactcatccttatcagcttctttgagcccttcaataacaaaagaggcctttgccctgagccaatcagtgagggcagcctttgattgagcatcttcagaccttctttcttcctccaactttatgtgcttactcagaccttcaacttttttatctattctttcccatgcctcatcataaactttgccccaatcttcaccgtgtttcttttcaaggtgaagcaaaaggtcaccaaactgataacgcgcaaacttttttccttttttacggtcttcttcagacgaaagatatggaagcaaggcttcctgccttttatatccagcaagattttgccagaagaccttcccgtcctctttcttttcgttaatcaactttttgacattacagaccatatcccaccaatcaacctcattcgcctggcgttcaacaagagggaaggacggaaaacccttaagccgctgtaagggctttgcctcatccctgccaattttgagtttctgccaaagattcaggtttacccagatcactatctgagcaacaacattgttataagcttcaatcccttcttttgtatgcggttgcggtggaagagtgattttaggaaatgcaagcccgtttgcacttgctatatcctttagatttgccaatctcttttcgtttttttttataaccttttggtgttcgaggatgatgtcctggtactttgtaaggaaactggctactgctcccatacaggcatcagataaagccttaccaacgggaccacttgcgcagctattgccaccgatctgttctagcggctttacaggatggttcgattctcttgttacgtggattgaataaaagtccaatgccctttgaccgaacttccccaacgaatacgttactagctcgtcatttgcctccggtttatgcggcgagagcaatatcaaacgttcatgctcggagacattacaacggccaaagtaatttgtatggggcttacccttgtcattcacttgttcaagcttataaacatagaggggttgacagcactgagaacaggcaaatccagaacttgttagtctctcatttccgtccttcaccggaatcaattttctctgatcaatattcttgggcgctggttgtgcaaccctgctcatcaatccgacagggtctttttggaactcttcccaataaacatgcaggattgctttcttcatttccgtatagtcagtgaggagtttatttaaatttgcacgtgaagtatttgaaatgggctgaggaatgttttccggctttttgcgaagattctctaacctttctctcaggtcaggtgtcataacccgaacgagcaaggttttcatagggccggttttgccggcttttttcgtgttgctatcctttaccaatctccttcgtattttatttatcctttttatttcctgcatcttt

SEQ ID NO: 15 - protein, 986 aa, Deltaproteobacteria bacterium
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLNSNSTEFKSYKSGKQPFVGAWQAFYKRRLKEVWKPNA

SEQ ID NO: 16 - DNA, 2962 bp, Deltaproteobacteria bacterium
atggaaaagagaataaacaagatacgaaagaaactatcggccgataatgccacaaagcctgtgagcaggagcggccccatgaaaacactccttgtccgggtcatgacggacgacttgaaaaaaagactggagaagcgtcggaaaaagccggaagttatgccgcaggttatttcaaataacgcagcaaacaatcttagaatgctccttgatgactatacaaagatgaaggaggcgatactacaagtttactggcaggaatttaaggacgaccatgtgggcttgatgtgcaaatttgcccagcctgcttccaaaaaaattgaccagaacaaactaaaaccggaaatggatgaaaaaggaaatctaacaactgccggttttgcatgttctcaatgcggtcagccgctatttgtttataagcttgaacaggtgagtgaaaaaggcaaggcttatacaaattacttcggccggtgtaatgtggccgagcatgagaaattgattcttcttgctcaattaaaacctgaaaaagacagtgacgaagcagtgacatactcccttggcaaattcggccagagggcattggacttttattcaatccacgtaacaaaagaatccacccatccagtaaagcccctggcacagattgcgggcaaccgctatgcaagcggacctgttggcaaggccctttccgatgcctgtatgggcactatagccagttttctttcgaaatatcaagacatcatcatagaacatcaaaaggttgtgaagggtaatcaaaagaggttagagagtctcagggaattggcagggaaagaaaatcttgagtacccatcggttacactgccgccgcagccgcatacgaaagaaggggttgacgcttataacgaagttattgcaagggtacgtatgtgggttaatcttaatctgtggcaaaagctgaagctcagccgtgatgacgcaaaaccgctactgcggctaaaaggattcccatctttccctgttgtggagcggcgtgaaaacgaagttgactggtggaatacgattaatgaagtaaaaaaactgattgacgctaaacgagatatgggacgggtattctggagcggcgttaccgcagaaaagagaaataccatccttgaaggatacaactatctgccaaatgagaatgaccataaaaagagagagggcagtttggaaaaccctaagaagcctgccaaacgccagtttggagacctcttgctgtatcttgaaaagaaatatgccggagactggggaaaggtcttcgatgaggcatgggagaggatagataagaaaatagccggactcacaagccatatagagcgcgaagaagcaagaaacgcggaagacgctcaatccaaagccgtacttacagactggctaagggcaaaggcatcatttgttcttgaaagactgaaggaaatggatgaaaaggaattctatgcgtgtgaaatccaacttcaaaaatggtatggcgatcttcgaggcaacccgtttgccgttgaagctgagaatagagttgttgatataagcgggttttctatcggaagcgatggccattcaatccaatacagaaatctccttgcctggaaatatctggagaacggcaagcgtgaattctatctgttaatgaattatggcaagaaagggcgcatcagatttacagatggaacagatattaaaaagagcggcaaatggcagggactattatatggcggtggcaaggcaaaggttattgatctgactttcgaccccgatgatgaacagttgataatcctgccgctggcctttggcacaaggcaaggccgcgagtttatctggaacgatttgctgagtcttgaaacaggcctgataaagctcgcaaacggaagagttatcgaaaaaacaatctataacaaaaaaatagggcgggatgaaccggctctattcgttgccttaacatttgagcgccgggaagttgttgatccatcaaatataaagcctgtaaaccttataggcgttgaccgcggcgaaaacatcccggcggttattgcattgacagaccctgaaggttgtcctttaccggaattcaaggattcatcagggggcccaacagacatcctgcgaataggagaaggatataaggaaaagcagagggctattcaggcagcaaaggaggtagagcaaaggcgggctggcggttattcacggaagtttgcatccaagtcgaggaacctggcggacgacatggtgagaaattcagcgcgagaccttttttaccatgccgttacccacgatgccgtccttgtctttgaaaacctgagcaggggttttggaaggcagggcaaaaggaccttcatgacggaaagacaatatacaaagatggaagactggctgacagcgaagctcgcatacgaaggtcttacgtcaaaaacctacctttcaaagacgctggcgcaatatacgtcaaaaacatgctccaactgcgggtttactataacgactgccgattatgacgggatgttggtaaggcttaaaaagacttctgatggatgggcaactaccctcaacaacaaagaattaaaagccgaaggccagataacgtattataaccggtataaaaggcaaaccgtggaaaaagaactctccgcagagcttgacaggctttcagaagagtcgggcaataatgatatttctaagtggaccaagggtcgccgggacgaggcattatttttgttaaagaaaagattcagccatcggcctgttcaggaacagtttgtttgcctcgattgcggccatgaagtccacgccgatgaacaggcagccttgaatattgcaaggtcatggctttttctaaactcaaattcaacagaattcaaaagttataaatcgggtaaacagcccttcgttggtgcttggcaggccttttacaaaaggaggcttaaagaggtatggaagcccaacgcctgat

SEQ ID NO: 17 - protein, 949 aa, Candidatus Micrarchaeum acidiphilum
MRDSITAPRYSSALAARIKEFNSAFKLGIDLGTKTGGVALVKDNKVLLAKTFLDYHKQTLEERRIHRRNRRSRLARRKRIARLRSWILRQKIYGKQLPDPYKIKKMQLPNGVRKGENWIDLVVSGRDLSPEAFVRAITLIFQKRGQRYEEVAKEIEEMSYKEFSTHIKALTSVTEEEFTALAAEIERRQDVVDTDKEAERYTQLSELLSKVSESKSESKDRAQRKEDLGKVVNAFCSAHRIEDKDKWCKELMKLLDRPVRHARFLNKVLIRCNICDRATPKKSRPDVRELLYFDTVRNFLKAGRVEQNPDVISYYKKIYMDAEVIRVKILNKEKLTDEDKKQKRKLASELNRYKNKEYVTDAQKKMQEQLKTLLFMKLTGRSRYCMAHLKERAAGKDVEEGLHGVVQKRHDRNIAQRNHDLRVINLIESLLFDQNKSLSDAIRKNGLMYVTIEAPEPKTKHAKKGAAVVRDPRKLKEKLFDDQNGVCIYTGLQLDKLEISKYEKDHIFPDSRDGPSIRDNLVLTTKEINSDKGDRTPWEWMHDNPEKWKAFERRVAEFYKKGRINERKRELLLNKGTEYPGDNPTELARGGARVNNFITEFNDRLKTHGVQELQTIFERNKPIVQVVRGEETQRLRRQWNALNQNFIPLKDRAMSFNHAEDAAIAASMPPKFWREQIYRTAWHFGPSGNERPDFALAELAPQWNDFFMTKGGPIIAVLGKTKYSWKHSIIDDTIYKPFSKSAYYVGIYKKPNAITSNAIKVLRPKLLNGEHTMSKNAKYYHQKIGNERFLMKSQKGGSIITVKPHDGPEKVLQISPTYECAVLTKHDGKIIVKFKPIKPLRDMYARGVIKAMDKELETSLSSMSKHAKYKELHTHDIIYLPATKKHVDGYFIITKLSAKHGIKALPESMVKVKYTQIGSENNSEVKLTKPKPEITLDSEDITNIYNFTR

SEQ ID NO: 18 - DNA, 2851 bp, Candidatus Micrarchaeum acidiphilum
atgagagactctattactgcacctagatacagctccgctcttgccgccagaataaaggagtttaattctgctttcaagttaggaatcgacctaggaacaaaaaccggcggcgtagcactggtaaaagacaacaaagtgctgctcgctaagacattcctcgattaccataaacaaacactggaggaaaggaggatccatagaagaaacagaaggagcaggctagccaggcggaagaggattgctcggctgcgatcatggatactcagacagaagatttatggcaagcagcttcctgacccatacaaaatcaaaaaaatgcagttgcctaatggtgtacgaaaaggggaaaactggattgacctggtagtttctggacgggacctttcaccagaagccttcgtgcgtgcaataactctgatattccaaaagagagggcaaagatatgaagaagtggccaaagagatagaagaaatgagttacaaggaatttagtactcacataaaagccctgacatccgttactgaagaagaatttactgctctggcagcagagatagaacggaggcaggatgtggttgacacagacaaggaggccgaacgctatacccaattgtctgagttgctctccaaggtctcagaaagcaaatctgaatctaaagacagagcgcagcgtaaggaggatctcggaaaggtggtgaacgctttctgcagtgctcatcgtatcgaagacaaggataaatggtgtaaagaacttatgaaattactagacagaccagtcagacacgctaggttccttaacaaagtactgatacgttgcaatatctgcgatagggcaacccctaagaaatccagacctgacgtgagggaactgctatattttgacacagtaagaaacttcttgaaggctggaagagtggagcaaaacccagacgttattagttactataaaaaaatttatatggatgcagaagtaatcagggtcaaaattctgaataaggaaaagctgactgatgaggacaaaaagcaaaagaggaaattagcgagcgaacttaacaggtacaaaaacaaagaatacgtgactgatgcgcagaagaagatgcaagagcaacttaagacattgctgttcatgaagctgacaggcaggtctagatactgcatggctcatcttaaggaaagggcagcaggcaaagatgtagaagaaggacttcatggcgttgtgcagaaaagacacgacaggaacatagcacagcgcaatcacgacttacgtgtgattaatcttattgagagtctgcttttcgaccaaaacaaatcgctctccgatgcaataaggaagaacgggttaatgtatgttactattgaggctccagagccaaagactaagcacgcaaagaaaggcgcagctgtggtaagggatcccagaaagttgaaggagaagttgtttgatgatcaaaacggcgtttgcatatatacgggcttgcagttagacaaattagagataagtaaatacgagaaggaccatatctttccagattcaagggatggaccatctatcagggacaatcttgtactcactacaaaagagataaattcagacaaaggcgataggaccccatgggaatggatgcatgataacccagaaaaatggaaagcgttcgagagaagagtcgcagaattctataagaaaggcagaataaatgagaggaaaagagaactcctattaaacaaaggcactgaataccctggcgataacccgactgagctggcgcggggaggcgcccgtgttaacaactttattactgaatttaatgaccgcctcaaaacgcatggagtccaggaactgcagaccatctttgagcgtaacaaaccaatagtgcaggtagtcaggggtgaagaaacgcagcgtctgcgcagacaatggaatgcactaaaccagaatttcataccactaaaggacagggcaatgtcgttcaaccacgctgaagacgcagccatagcagcaagcatgccaccaaaattctggagggagcagatataccgtactgcgtggcactttggacctagtggaaatgagagaccggactttgctttggcagaattggcgccacaatggaatgacttctttatgactaagggcggtccaataatagcagtgctgggcaaaacgaagtatagttggaagcacagcataattgatgacactatatacaagccattcagcaaaagtgcttactatgttgggatatacaaaaagccgaacgccatcacgtccaatgctataaaagtcttaaggccaaaactcttaaatggcgaacatacaatgtctaagaatgcaaagtattatcatcagaagattggtaatgagcgcttcctcatgaaatctcagaaaggtggatcgataattacagtaaaaccacacgacggaccggaaaaagtgcttcaaatcagccctacatatgaatgcgcagtccttactaagcatgacggtaaaataatagtcaaatttaaaccaataaagccgctacgggacatgtatgcccgcggtgtgattaaagccatggacaaagagcttgaaacaagcctctctagcatgagtaaacacgctaagtacaaggagttacacactcatgatatcatatatctgcctgctacaaagaagcacgtagatggctacttcataataaccaaactaagtgcgaaacatggcataaaagcactccccgaaagcatggttaaagtcaagtatactcaaattgggagtgaaaacaatagtgaagtgaagcttaccaaaccaaaaccagagataactttggatagtgaagatattacaaacatatataatttcacccgctaag

SEQ ID NO: 19 - protein, 967 aa, Candidatus Parvarchaeum acidiphilum
MLGSSRYLRYNLTSFEGKEPFLIMGYYKEYNKELSSKAQKEFNDQISEFNSYYKLGIDLGDKTGIAIVKGNKIILAKTLIDLHSQKLDKRREARRNRRTRLSRKKRLARLRSWVMRQKVGNQRLPDPYKIMHDNKYWSIYNKSNSANKKNWIDLLIHSNSLSADDFVRGLTIIFRKRGYLAFKYLSRLSDKEFEKYIDNLKPPISKYEYDEDLEELSSRVENGEIEEKKFEGLKNKLDKIDKESKDFQVKQREEVKKELEDLVDLFAKSVDNKIDKARWKRELNNLLDKKVRKIRFDNRFILKCKIKGCNKNTPKKEKVRDFELKMVLNNARSDYQISDEDLNSFRNEVINIFQKKENLKKGELKGVTIEDLRKQLNKTFNKAKIKKGIREQIRSIVFEKISGRSKFCKEHLKEFSEKPAPSDRINYGVNSAREQHDFRVLNFIDKKIFKDKLIDPSKLRYITIESPEPETEKLEKGQISEKSFETLKEKLAKETGGIDIYTGEKLKKDFEIEHIFPRARMGPSIRENEVASNLETNKEKADRTPWEWFGQDEKRWSEFEKRVNSLYSKKKISERKREILLNKSNEYPGLNPTELSRIPSTLSDFVESIRKMFVKYGYEEPQTLVQKGKPIIQVVRGRDTQALRWRWHALDSNIIPEKDRKSSFNHAEDAVIAACMPPYYLRQKIFREEAKIKRKVSNKEKEVTRPDMPTKKIAPNWSEFMKTRNEPVIEVIGKVKPSWKNSIMDQTFYKYLLKPFKDNLIKIPNVKNTYKWIGVNGQTDSLSLPSKVLSISNKKVDSSTVLLVHDKKGGKRNWVPKSIGGLLVYITPKDGPKRIVQVKPATQGLLIYRNEDGRVDAVREFINPVIEMYNNGKLAFVEKENEEELLKYFNLLEKGQKFERIRRYDMITYNSKFYYVTKINKNHRVTIQEESKIKAESDKVKSSSGKEYTRKETEELSLQKLAELISI

SEQ ID NO: 20 - DNA, 2906 bp, Candidatus Parvarchaeum acidiphilum
atgttaggctccagcaggtacctccgttataacctaacctcgtttgaaggcaaggagccatttttaataatgggatattacaaagagtataataaggaattaagttccaaagctcaaaaagaatttaatgatcaaatttctgaatttaattcgtattacaaactaggtatagatctcggagataaaacaggaattgcaatcgtaaagggcaacaaaataatcctagcaaaaacactaattgatttgcattcccaaaaattagataaaagaagggaagctagaagaaatagaagaactcggctttccagaaagaaaaggcttgcgagattaagatcgtgggtaatgcgtcagaaagttggcaatcaaagacttcccgatccatataaaataatgcatgacaataagtactggtctatatataataagagtaattctgcaaataaaaagaattggatagatctgttaatccacagtaactctttatcagcagacgattttgttagaggcttaactataattttcagaaaaagaggctatttagcatttaagtatctttcaaggttaagcgataaggaatttgaaaaatacatagataacttaaaaccacctataagcaaatacgagtatgatgaggatttagaagaattatcaagcagggttgaaaatggggaaatagaggaaaagaaattcgaaggcttaaagaataagctagataaaatagacaaagaatctaaagactttcaagtaaagcaaagagaagaagtaaaaaaggaactggaagacttagttgatttgtttgctaaatcagttgataataaaatagataaagctaggtggaaaagggagctaaataatttattggataagaaagtaaggaaaatacggtttgacaaccgctttattttgaagtgcaaaattaagggctgtaacaagaatactccaaagaaagagaaggtcagagattttgaattgaagatggttttaaataatgctagaagcgattatcagatttctgatgaggatttaaactcttttagaaatgaagtaataaatatatttcaaaagaaggaaaacttaaagaaaggagagctgaaaggagttactattgaagatttgagaaagcagcttaataaaacttttaataaagccaagattaaaaaagggataagggagcagataaggtctatcgtgtttgaaaaaattagtggaaggagtaaattctgcaaagaacatctaaaagaattttctgagaagccggctccttctgacaggattaattatggggttaattcagcaagagaacaacatgattttagagtcttaaatttcatagataaaaaaatattcaaagataagttgatagatccctcaaaattgaggtatataactattgaatctccagaaccagaaacagagaagttggaaaaaggtcaaatatcagagaagagcttcgaaacattgaaagaaaaattggctaaagaaacaggtggtattgatatatacactggtgaaaaattaaagaaagactttgaaatagagcacatattcccaagagcaaggatggggccttctataagggaaaacgaagtagcatcaaatctggaaacaaataaggaaaaggccgatagaactccttgggaatggtttgggcaagatgaaaaaagatggtcagagtttgagaaaagagttaattctctttatagtaaaaagaaaatatcagagagaaaaagagaaattttgttaaataagagtaatgaatatccgggattaaaccctacagaactaagtagaatacctagtacgctgagcgacttcgttgagagtataagaaaaatgtttgttaagtatggctatgaagagcctcaaactttggttcaaaaaggaaaaccgataatacaagttgttagaggcagagacacacaagctttgaggtggagatggcatgcattagatagtaatataataccagaaaaggacaggaaaagttcatttaatcacgctgaagatgcagttattgccgcctgtatgccaccttactatctcaggcaaaaaatatttagagaagaagcaaaaataaaaagaaaagtaagcaataaggaaaaggaagttacacggcctgacatgcctactaaaaagatagctccgaactggtcggaatttatgaaaactagaaatgagccggttattgaagtaataggaaaagttaagccaagctggaaaaacagcataatggatcaaacattttataaatatcttttgaagccatttaaagataacctgataaaaatacccaacgttaaaaatacatacaagtggataggagttaatggacaaactgattcattatccctcccgagtaaggtcttatctatctctaataaaaaggttgattcttctacagttcttcttgtgcatgataagaagggtggtaagcggaattgggtacctaaaagtatagggggtttgttggtatatataactcctaaagacgggccgaaaagaatagttcaagtaaagccagcaactcagggtttgttaatatatagaaatgaagatggcagagtagatgctgtaagagagttcataaatccagtgatagaaatgtataataatggcaaattggcatttgtagaaaaagaaaatgaagaagagcttttgaaatattttaatttgctggaaaaaggtcaaaaatttgaaagaataagacggtatgatatgataacctacaatagtaaattttactatgtaacaaaaataaacaagaatcacagagttactatacaagaagagtctaagataaaagcagaatcagacaaagttaagtcctcttcaggcaaagagtatactcgtaaggaaaccgaggaattatcacttcaaaaattagcggaattaattagtatataaaa

SEQ ID NO: 21 - DNA, 21 bp, Artificial Sequence
  Description of Artificial Sequence Synthetic oligonucleotide
gcagaactacacaccagggcc

SEQ ID NO: 22 - DNA, 21 bp, Artificial Sequence
  Description of Artificial Sequence Synthetic oligonucleotide
ggatagatgtaaaagacacca

SEQ ID NO: 23 - DNA, 21 bp, Artificial Sequence
  Description of Artificial Sequence Synthetic oligonucleotide
gcggcagcatagtgagcccag

SEQ ID NO: 24 - DNA, 20 bp, Artificial Sequence
  Description of Artificial Sequence Synthetic oligonucleotide
tcagtttacacccgatccac

SEQ ID NO: 25 - DNA, 36 bp, Artificial Sequence
  Description of Artificial Sequence Synthetic oligonucleotide
aaacagggccagggatcagatatccactgaccttgt

SEQ ID NO: 26 - DNA, 35 bp, Artificial Sequence
  Description of Artificial Sequence Synthetic oligonucleotide
taaacaaggtcagtggatatctgatccctggccct

SEQ ID NO: 27 - DNA, 36 bp, Artificial Sequence
  Description of Artificial Sequence Synthetic oligonucleotide
aaacagctcgatgtcagcagttcttgaagtactcgt

SEQ ID NO: 28 - DNA, 35 bp, Artificial Sequence
  Description of Artificial Sequence Synthetic oligonucleotide
taaacgagtacttcaagaactgctgacatcgagct

SEQ ID NO: 29 - DNA, 24 bp, Artificial Sequence
  Description of Artificial Sequence Synthetic oligonucleotide
caccgattggcagaactacacacc

SEQ ID NO: 30 - DNA, 24 bp, Artificial Sequence
  Description of Artificial Sequence Synthetic oligonucleotide
aaacggtgtgtagttctgccaatc

SEQ ID NO: 31 - DNA, 24 bp, Artificial Sequence
  Description of Artificial Sequence Synthetic oligonucleotide
caccgcgtggcctgggcgggactg

SEQ ID NO: 32 - DNA, 24 bp, Artificial Sequence
  Description of Artificial Sequence Synthetic oligonucleotide
aaaccagtcccgcccaggccacgc

SEQ ID NO: 33 - DNA, 27 bp, Artificial Sequence
  Description of Artificial Sequence Synthetic oligonucleotide
caccgatctgtggatctaccacacaca

SEQ ID NO: 34 - DNA, 27 bp, Artificial Sequence
  Description of Artificial Sequence Synthetic oligonucleotide
aaactgtgtgtggtagatccacagatc

SEQ ID NO: 35 - DNA, 27 bp, Artificial Sequence
  Description of Artificial Sequence Synthetic oligonucleotide
caccgctgcttatatgcagcatctgag

SEQ ID NO: 36 - DNA, 27 bp, Artificial Sequence
  Description of Artificial Sequence Synthetic oligonucleotide
aaacctcagatgctgcatataagcagc

SEQ ID NO: 37 - DNA, 25 bp, Artificial Sequence
  Description of Artificial Sequence Synthetic oligonucleotide
caccgtgtggtagatccacagatca

SEQ ID NO: 38 - DNA, 25 bp, Artificial Sequence
  Description of Artificial Sequence Synthetic oligonucleotide
aaactgatctgtggatctaccacac

SEQ ID NO: 39 - DNA, 25 bp, Artificial Sequence
  Description of Artificial Sequence Synthetic oligonucleotide
caccgcagggaagtagccttgtgtg

SEQ ID NO: 40 - DNA, 25 bp, Artificial Sequence
  Description of Artificial Sequence Synthetic oligonucleotide
aaaccacacaaggctacttccctgc

SEQ ID NO: 41 - DNA, 25 bp, Artificial Sequence
  Description of Artificial Sequence Synthetic oligonucleotide
caccgatcagatatccactgacctt

SEQ ID NO: 42 - DNA, 25 bp, Artificial Sequence
  Description of Artificial Sequence Synthetic oligonucleotide
aaacaaggtcagtggatatctgatc

SEQ ID NO: 43 - DNA, 25 bp, Artificial Sequence
  Description of Artificial Sequence Synthetic oligonucleotide
caccgcacactaatacttctccctc

SEQ ID NO: 44 - DNA, 25 bp, Artificial Sequence
  Description of Artificial Sequence Synthetic oligonucleotide
aaacgagggagaagtattagtgtgc

SEQ ID NO: 45 - DNA, 25 bp, Artificial Sequence
  Description of Artificial Sequence Synthetic oligonucleotide
caccgcctcctagcatttcgtcaca

SEQ ID NO: 46 - DNA, 25 bp, Artificial Sequence
  Description of Artificial Sequence Synthetic oligonucleotide
aaactgtgacgaaatgctaggaggc

SEQ ID NO: 47 - DNA, 25 bp, Artificial Sequence
  Description of Artificial Sequence Synthetic oligonucleotide
caccgcatggcccgagagctgcatc

SEQ ID NO: 48 - DNA, 25 bp, Artificial Sequence
  Description of Artificial Sequence Synthetic oligonucleotide
aaacgatgcagctctcgggccatgc

SEQ ID NO: 49 - DNA, 25 bp, Artificial Sequence
  Description of Artificial Sequence Synthetic oligonucleotide
caccgcagcagtctttgtagtactc

SEQ ID NO: 50 - DNA, 25 bp, Artificial Sequence
  Description of Artificial Sequence Synthetic oligonucleotide
aaacgagtactacaaagactgctgc

SEQ ID NO: 51 - DNA, 25 bp, Artificial Sequence
  Description of Artificial Sequence Synthetic oligonucleotide
caccgctgacatcgagctttctaca

SEQ ID NO: 52 - DNA, 25 bp, Artificial Sequence
  Description of Artificial Sequence Synthetic oligonucleotide
aaactgtagaaagctcgatgtcagc

SEQ ID NO: 53 - DNA, 25 bp, Artificial Sequence
  Description of Artificial Sequence Synthetic oligonucleotide
caccgtctacaagggactttccgct

SEQ ID NO: 54 - DNA, 25 bp, Artificial Sequence
  Description of Artificial Sequence Synthetic oligonucleotide
aaacagcggaaagtcccttgtagac

SEQ ID NO: 55 - DNA, 25 bp, Artificial Sequence
  Description of Artificial Sequence Synthetic oligonucleotide
caccgctttccgctggggactttcc

SEQ ID NO: 56 - DNA, 25 bp, Artificial Sequence
  Description of Artificial Sequence Synthetic oligonucleotide
aaacggaaagtccccagcggaaagc

SEQ ID NO: 57 - DNA, 25 bp, Artificial Sequence
  Description of Artificial Sequence Synthetic oligonucleotide
caccgcctccctggaaagtccccag

SEQ ID NO: 58 - DNA, 25 bp, Artificial Sequence
  Description of Artificial Sequence Synthetic oligonucleotide
aaacctggggactttccagggaggc

SEQ ID NO: 59 - DNA, 24 bp, Artificial Sequence
  Description of Artificial Sequence Synthetic oligonucleotide
caccgcctgggcgggactggggag

SEQ ID NO: 60 - DNA, 24 bp, Artificial Sequence
  Description of Artificial Sequence Synthetic oligonucleotide
aaacctccccagtcccgcccaggc

SEQ ID NO: 61 - DNA, 25 bp, Artificial Sequence
  Description of Artificial Sequence Synthetic oligonucleotide
caccgtccatcccatgcaggctcac

SEQ ID NO: 62 - DNA, 25 bp, Artificial Sequence
  Description of Artificial Sequence Synthetic oligonucleotide
aaacgtgagcctgcatgggatggac

SEQ ID NO: 63 - DNA, 25 bp, Artificial Sequence
  Description of Artificial Sequence Synthetic oligonucleotide
caccgcggagagagaagtattagag

SEQ ID NO: 64 - DNA, 25 bp, Artificial Sequence
  Description of Artificial Sequence Synthetic oligonucleotide
aaacctctaatacttctctctccgc

SEQ ID NO: 65 - DNA, 24 bp, Artificial Sequence
  Description of Artificial Sequence Synthetic oligonucleotide
caccggccagatgagagaaccaag

SEQ ID NO: 66 - DNA, 24 bp, Artificial Sequence
  Description of Artificial Sequence Synthetic oligonucleotide
aaaccttggttctctcatctggcc

SEQ ID NO: 67 - DNA, 26 bp, Artificial Sequence
  Description of Artificial Sequence Synthetic oligonucleotide
caccgccttcccacaagggaaggcca

SEQ ID NO: 68 - DNA, 26 bp, Artificial Sequence
  Description of Artificial Sequence Synthetic oligonucleotide
aaactggccttcccttgtgggaaggc

SEQ ID NO: 69 - DNA, 26 bp, Artificial Sequence
  Description of Artificial Sequence Synthetic oligonucleotide
caccgcgagagcgtcggtattaagcg

SEQ ID NO: 70 - DNA, 26 bp, Artificial Sequence
  Description of Artificial Sequence Synthetic oligonucleotide
aaaccgcttaataccgacgctctcgc

SEQ ID NO: 71 - DNA, 25 bp, Artificial Sequence
  Description of Artificial Sequence Synthetic oligonucleotide
caccggatagatgtaaaagacacca

SEQ ID NO: 72 - DNA, 25 bp, Artificial Sequence
  Description of Artificial Sequence Synthetic oligonucleotide
aaactggtgtcttttacatctatcc

SEQ ID NO: 73 - DNA, 24 bp, Artificial Sequence
  Description of Artificial Sequence Synthetic oligonucleotide
caccgcaggatatgtaactgacag

SEQ ID NO: 74 - DNA, 24 bp, Artificial Sequence
  Description of Artificial Sequence Synthetic oligonucleotide
aaacctgtcagttacatatcctgc

SEQ ID NO: 75 - DNA, 24 bp, Artificial Sequence
  Description of Artificial Sequence Synthetic oligonucleotide
caccgcatgggtaccagcacacaa

SEQ ID NO: 76 - DNA, 24 bp, Artificial Sequence
  Description of Artificial Sequence Synthetic oligonucleotide
aaacttgtgtgctggtacccatgc

SEQ ID NO: 77 - DNA, 25 bp, Artificial Sequence
  Description of Artificial Sequence Synthetic oligonucleotide
caccgctttattgaggcttaagcag

SEQ ID NO: 78 - DNA, 24 bp, Artificial Sequence
  Description of Artificial Sequence Synthetic oligonucleotide
aaacgagtcacacaacagacgggc

SEQ ID NO: 79 - DNA, 25 bp, Artificial Sequence
  Description of Artificial Sequence Synthetic oligonucleotide
tggaatgcagtggcgcgatcttggc

SEQ ID NO: 80 - DNA, 23 bp, Artificial Sequence
  Description of Artificial Sequence Synthetic oligonucleotide
cacagcatcaagaagaacctgat

SEQ ID NO: 81 - DNA, 23 bp, Artificial Sequence
  Description of Artificial Sequence Synthetic oligonucleotide
tgaagatctcttgcagatagcag

SEQ ID NO: 82 - DNA, 40 bp, Homo sapiens
cttcttactgtccccttctgggctcactatgctgccgccc

SEQ ID NO: 83 - DNA, 40 bp, Artificial Sequence; identical to SEQ ID NO: 82
  Description of Artificial Sequence Synthetic oligonucleotide

SEQ ID NO: 84 - DNA, 40 bp, Artificial Sequence; identical to SEQ ID NO: 82
  Description of Artificial Sequence Synthetic oligonucleotide

SEQ ID NO: 85 - DNA, 38 bp, Artificial Sequence
  Description of Artificial Sequence Synthetic oligonucleotide
  modified_base (15)..(15): a, c, t, g, unknown or other
cttcttactgtcccnttctgctcactatgctgccgccc

SEQ ID NO: 86 - DNA, 39 bp, Artificial Sequence
  Description of Artificial Sequence Synthetic oligonucleotide
  modified_base (16)..(16): a, c, t, g, unknown or other
cttcttactgtccccnttcggctcactatgctgccgccc

SEQ ID NO: 87 - DNA, 38 bp, Artificial Sequence
  Description of Artificial Sequence Synthetic oligonucleotide
cttcttactgtccccttcggctcactatgctgccgccc

SEQ ID NO: 88 - DNA, 38 bp, Artificial Sequence
  Description of Artificial Sequence Synthetic oligonucleotide
cttcttactgtcccttctggctcactatgctgccgccc

SEQ ID NO: 89 - DNA, 40 bp, Homo sapiens; identical to SEQ ID NO: 82

SEQ ID NO: 90 - DNA, 20 bp, Artificial Sequence
  Description of Artificial Sequence Synthetic oligonucleotide
cttcttactgtccccttctg

SEQ ID NO: 91 - DNA, 20 bp, Artificial Sequence
  Description of Artificial Sequence Synthetic oligonucleotide
cttcttactgtccccttctg

SEQ ID NO: 92 - DNA, 20 bp, Artificial Sequence
  Description of Artificial Sequence Synthetic oligonucleotide
cactggggagcaggaaatat

SEQ ID NO: 93 - DNA, 20 bp, Artificial Sequence
  Description of Artificial Sequence Synthetic oligonucleotide
cttcttactgtccccttctg

SEQ ID NO: 94 - DNA, 27 bp, Human immunodeficiency virus
tggaagggctaatttggtcccaaaaaa

SEQ ID NO: 95 - DNA, 27 bp, Artificial Sequence
  Description of Artificial Sequence Synthetic oligonucleotide
tggaagggctaattcactcccaacgaa

SEQ ID NO: 96 - DNA, 27 bp, Artificial Sequence
  Description of Artificial Sequence Synthetic oligonucleotide
tggaagggctaattcactcccaacgaa

SEQ ID NO: 97 - DNA, 27 bp, Artificial Sequence
  Description of Artificial Sequence Synthetic oligonucleotide
tggaagggctaattcactcccaacgaa

SEQ ID NO: 98 - DNA, 27 bp, Artificial Sequence
  Description of Artificial Sequence Synthetic oligonucleotide
tggaagggctaattcactcccaacgaa

SEQ ID NO: 99 - DNA, 27 bp, Artificial Sequence
  Description of Artificial Sequence Synthetic oligonucleotide
tggaagggctaattcactcccaacgaa

SEQ ID NO: 100 - DNA, 27 bp, Artificial Sequence
  Description of Artificial Sequence Synthetic oligonucleotide
tggaagggctaattcactcccaacgaa

SEQ ID NO: 101 - DNA, 27 bp, Artificial Sequence
  Description of Artificial Sequence Synthetic oligonucleotide
tggaagggctaattcactcccaacgaa

SEQ ID NO: 102 - DNA, 27 bp, Artificial Sequence
  Description of Artificial Sequence Synthetic oligonucleotide
tggaagggctaattcactcccaacgaa

SEQ ID NO: 103 - DNA, 27 bp, Artificial Sequence
  Description of Artificial Sequence Synthetic oligonucleotide
tggaagggctaattcactcccaacgaa

SEQ ID NO: 104 - DNA, 27 bp, Artificial Sequence
  Description of Artificial Sequence Synthetic oligonucleotide
tggaagggctaattcactcccaacgaa

SEQ ID NO: 105 - DNA, 27 bp, Artificial Sequence
  Description of Artificial Sequence Synthetic oligonucleotide
tggaagggctaattcactcccaacgaa

SEQ ID NO: 106 - DNA, 27 bp, Artificial Sequence
  Description of Artificial Sequence Synthetic oligonucleotide
tggaagggctaattcactcccaacgaa

SEQ ID NO: 107 - DNA, 27 bp, Artificial Sequence
  Description of Artificial Sequence Synthetic oligonucleotide
tggaagggctaattcactcccaacgaa

SEQ ID NO: 108 - DNA, 118 bp, Artificial Sequence
  Description of Artificial Sequence Synthetic polynucleotide
caatggaaagtccctattggcgttactatgggaacatacgtcattattgacgtcaatgggcgggggtcgttgggcggtcagccaggcgggccatttaccgtaagttatgtaacgcgga

SEQ ID NO: 109 - DNA, 119 bp, Artificial Sequence
  Description of Artificial Sequence Synthetic polynucleotide
actccatatatgggctatgaactaatgaccccgtaattgattactattaataactagtcaataatcaatgtcaacgcctcgagtctagaggccgcaggaacccctagtgatggagttgg

SEQ ID NO: 110 - DNA, 60 bp, Artificial Sequence
  Description of Artificial Sequence Synthetic oligonucleotide
ccactccctctctgcgcgctcgctcgctcactgaggccgggcgaccaaaggtcgcccggg

SEQ ID NO: 111 - DNA, 27 bp, Artificial Sequence
  Description of Artificial Sequence Synthetic oligonucleotide
tggaagggctaattcactcccaacgaa

SEQ ID NO: 112 - DNA, 27 bp, Artificial Sequence
  Description of Artificial Sequence Synthetic oligonucleotide
tggaagggctaattcactcccaacgaa

SEQ ID NO: 113 - DNA, 81 bp, Artificial Sequence
  Description of Artificial Sequence Synthetic oligonucleotide
aaaggtcgcccgacgcccgggcggcctcagtgagcgagcgagcgcgcagctgcctgcaggacatgtgagcaaaaggccagc

SEQ ID NO: 114 - DNA, 81 bp, Artificial Sequence
  Description of Artificial Sequence Synthetic oligonucleotide
aaaggtcgcccgacgcccgggcggcctcagtgagcgggcgagcgcgcagctgcctgcaggacatgtgagcaaaaggccagc

SEQ ID NO: 115 - DNA, 1090 bp, Human immunodeficiency virus
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagcct

SEQ ID NO: 116 - DNA, 1152 bp, Homo sapiens
cactttttatttatgcacagggtggaacaagatggattatcaagtgtcaagtccaatctatgacatcaatattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacaca

SEQ ID NO: 117 - DNA, 22 bp, Artificial Sequence
  Description of Artificial Sequence Synthetic primer
aaaatcaatgtgaagcaaatcg

SEQ ID NO: 118 - DNA, 21 bp, Artificial Sequence
  Description of Artificial Sequence Synthetic primer
ggaaatatctgtgggcttgtg

SEQ ID NO: 119 - DNA, 297 bp, Artificial Sequence
  Description of Artificial Sequence Synthetic polynucleotide
caatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcctcgagtctagaggccgcaggaacccctagtgatggagttggccactccctctctgcgcgctcgctcgctcactgaggccgggcgaccaaaggtcgcccggg

SEQ ID NO: 120 - DNA, 33 bp, Artificial Sequence
  Description of Artificial Sequence Synthetic oligonucleotide
ttggcagaactacacaccagggccagggatcag

SEQ ID NO: 121 - DNA, 31 bp, Artificial Sequence
  Description of Artificial Sequence Synthetic oligonucleotide
ttggcagaactacacaccagccagggatcag

SEQ ID NO: 122 - DNA, 32 bp, Artificial Sequence
  Description of Artificial Sequence Synthetic oligonucleotide
ttggcagaactacacaccaggccagggatcag

SEQ ID NO: 123 - DNA, 27 bp, Artificial Sequence
  Description of Artificial Sequence Synthetic oligonucleotide
ttggcagaactacacaccagggatcag

SEQ ID NO: 124 - DNA, 555 bp, Human immunodeficiency virus
ttggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 125 - DNA, 40 bp, Artificial Sequence
  Description of Artificial Sequence Synthetic oligonucleotide
agctcagtttacacccgatccactggggagcaggaaatat

SEQ ID NO: 126 - DNA, 37 bp, Artificial Sequence
  Description of Artificial Sequence Synthetic oligonucleotide
ttggcagaactacacaccagggccagggatcagatat

SEQ ID NO: 127 - DNA, 35 bp, Artificial Sequence
  Description of Artificial Sequence Synthetic oligonucleotide
ttggcagaactacacaccagccagggatcagatat

SEQ ID NO: 128 - DNA, 31 bp, Artificial Sequence
  Description of Artificial Sequence Synthetic oligonucleotide
ttggcagaactacacaccagggatcagatat

SEQ ID NO: 129 - DNA, 36 bp, Artificial Sequence
  Description of Artificial Sequence Synthetic oligonucleotide
ttggcagaactacacaccaggccagggatcagatat

SEQ ID NO: 130 - DNA, 24 bp, Artificial Sequence
  Description of Artificial Sequence Synthetic oligonucleotide
ttggcagaactacacaccaggtat

SEQ ID NO: 131 - DNA, 33 bp, Artificial Sequence
  Description of Artificial Sequence Synthetic oligonucleotide
ttggcagaactacacacgccagggatcagatat

SEQ ID NO: 132 - DNA, 27 bp, Human immunodeficiency virus
accagatctgagcctgggagctctctg

SEQ ID NO: 133 - DNA, 27 bp, Artificial Sequence
  Description of Artificial Sequence Synthetic oligonucleotide
accagatctgagcctgggagctctcgg

SEQ ID NO: 134 - DNA, 334 bp, Artificial Sequence
  Description of Artificial Sequence Synthetic polynucleotide
ttggcagaactacacaccaggcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcctcgagtctagaggccgcaggaacccctagtgatggagttggccactccctctctgcgcgctcgctcgctcactgaggccgggcgaccaaaggtcgcccggggccagggatcagatat

SEQ ID NO: 135 - DNA, 118 bp, Artificial Sequence
  Description of Artificial Sequence Synthetic polynucleotide
ttggcagaactacacaccaggaaaggtcgcccgacgcccgggcggcctcagtgagcgagcgagcgcgcagctgcctgcaggacatgtgagcaaaaggccagcgccagggatcagatat

SEQ ID NO: 136 - DNA, 118 bp, Artificial Sequence
  Description of Artificial Sequence Synthetic polynucleotide
ttggcagaactacacaccaggaaaggtcgcccgacgcccgggcggcctcagtgagcgggcgagcgcgcagctgcctgcaggacatgtgagcaaaaggccagcgccagggatcagatat

SEQ ID NO: 137 - DNA, 40 bp, Homo sapiens; identical to SEQ ID NO: 125

SEQ ID NO: 138 - DNA, 37 bp, Human immunodeficiency virus; identical to SEQ ID NO: 126

(19) United States
(12) Patent Application Publication
(10) Pub. No.: US 2019/0367924 A1
(43) Pub. Date: Dec. 5, 2019

(54) GENE EDITING THERAPY FOR HIV INFECTION VIA DUAL TARGETING OF HIV GENOME AND CCR5

(71) Applicant: Temple University - of the Commonwealth System of Higher Education, Philadelphia, PA (US)

(72) Inventors: Kamel Khalili, Bala Cynwyd, PA (US); Rafal Kaminski, Philadelphia, PA (US); Thomas Malcolm, Bedminster, NJ (US)

(73) Assignee: Temple University - of the Commonwealth System of Higher Education, Philadelphia, PA (US)

(21) Appl. No.: 16/486,799

(22) PCT Filed: Feb. 16, 2018
(86) PCT No.: PCT/US18/18516

§ 371 Date: Aug. 16, 2019

Related U.S. Application Data
(60) Provisional application No. 62/460,480, filed on Feb. 17, 2017.

(51) Int. Cl. C12N 15/113

(52) U.S. Cl. CPC C12N 15/1132; A61P 31/18; C12N 2310/20; C12N 15/1138

(57) ABSTRACT

Compositions for specifically cleaving target sequences in retroviruses include nucleic acids encoding a Clustered Regularly Interspace Short Palindromic Repeat (CRISPR) associated endonuclease and a guide RNA sequence complementary to a target sequence in a retrovirus and a receptor used by a retrovirus for infecting a cell. The CRISPR construct edits, for example, proviral HIV DNA, thereby eliminating the provirus from an infected cell and simultaneously edits a viral receptor, e.g. CCR5 preventing infection and reinfection of the host.

### CROSS REFERENCE TO RELATED APPLICATIONS

This Application claims the benefit of the priority of U.S. Provisional Application U.S. Patent Application No. 62/460,480 filed on Feb. 17, 2017, the entire contents of which are incorporated herein by reference in their entirety.

### STATEMENT REGARDING FEDERALLY SPONSORED RESEARCH

This invention was made with U.S. government support under a grant awarded by the National Institutes of Health (NIH) to Kamel Khalili (R01MH110360). The U.S. government may have certain rights in the invention.

### FIELD OF THE INVENTION

The present invention relates to compositions and methods that target a retroviral genome and a viral receptor, for example human immunodeficiency virus (HIV). The compositions, which can include nucleic acids encoding a Clustered Regularly Interspace Short Palindromic Repeat (CRISPR) associated endonuclease and a guide RNA sequence complementary to a target sequence in a human immunodeficiency virus and/or a viral receptor can be administered to a subject having or at risk for contracting an HIV infection.

### BACKGROUND

For more than three decades since the discovery of HIV-1, AIDS remains a major public health problem affecting greater than 35.3 million people worldwide. AIDS remains incurable due to the permanent integration of HIV-1 into the host genome. Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target the latently-infected cells, for example, resting memory T cells, brain macrophages, microglia, and astrocytes, gut-associated lymphoid cells, that serve as a reservoir for HIV-1. Persistent HIV-1 infection is also linked to co-morbidities including heart and renal diseases, osteopenia, and neurological disorders. There is a continuing need for curative therapeutic strategies that target persistent viral reservoirs.

Current therapy for controlling HIV-1 infection and preventing AIDS progression has dramatically decreased viral replication in cells susceptible to HIV-1 infection, but it does not eliminate the low level of viral replication in latently infected cells which contain integrated copies of HIV-1 proviral DNA. There is an urgent need for the development of for curative therapeutic strategies that target persistent viral reservoirs, including strategies for eradicating proviral DNA from the host cell genome.

### SUMMARY

The present invention provides compositions and methods relating to treatment and prevention of retroviral infections, for example, the human immunodeficiency virus HIV-1. The compositions and methods target the retroviral genome, a viral receptor or combinations thereof.

Specifically, the present invention provides compositions including a nucleic acid sequence encoding a CRISPR-associated endonuclease, and one or more isolated nucleic acid sequences encoding gRNAs, wherein each gRNA is complementary to a target sequence in a retroviral genome. In a preferred embodiment, two or more gRNAs are included in the composition, with each gRNA directing a Cas endonuclease to a different target site in integrated retroviral DNA. In some embodiments, at least one endonuclease targets a viral receptor, such as for example, CCR5 receptors. In another embodiment, a composition comprises two of more endonucleases targeted to a retroviral genome and two or more endonucleases targeted to a virus receptor.

In some embodiments, an expression vector comprises an isolated nucleic acid sequence encoding a CRISPR-associated endonuclease, and one or more isolated nucleic acid sequences encoding gRNAs, wherein each gRNA is complementary to a target sequence in a retroviral genome and/or a receptor used by a virus to attach to and/or infect a cell.

Other aspects are described infra.

Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIG. 1A is a schematic representation of a map of pCMV-SaCas9-HCgRNAs-kanamycin plasmid. Sequences for gRNAs (LTR1: SEQ ID NO: 21; gagD: SEQ ID NO: 22; CCR5 A: SEQ ID NO: 23; CCR5 B: SEQ ID NO: 24), embodied herein, are shown bottom of the figure. FIG. 1B is a schematic representation showing the sequences of the gRNAs targeting HIV sequences (HIV-1 NL4-3 sequence NCBI Ref. No.: AF324493.1; SEQ ID NO: 115) and the CCR5 receptor sequences (NCBI Ref. No.: NG_012637.1; SEQ ID NO: 116).

FIGS. 2A-2C show the CRISPR/Cas9 mediated disruption of human CCR5 gene in TZM-bl cells TZM-bl cells were co-transfected with pX601-HIV-1-LTR1-GagD-CCR5A-CCR5B and pKLV-BFP-PURO plasmids (ratio 5:1) and then selected with puromycin for 2 weeks. Single cell clones were screened by PCR for the presence of CRISPR/Cas9 double cleaved/end-joined truncated CCR5 gene products (FIG. 2A) which were purified and verified by Sanger sequencing (FIG. 2B; SEQ ID NOS: 82-93). Six of selected clones (two control and four CCR5 deletion mutants) were infected with different MOIs (0.01-1) of CCR5-tropic or control, VSV-g pseudotyped pan-tropic HIV-1-GFP reporter viruses. 48 h later viral expression was checked by GFP-FACS of paraformaldehyde fixed cells (FIG. 2C). CCR5-tropic virus failed to infect TZM-bl CCR5 gene mutated single cell clones.

FIGS. 3A-3C show the LTR-1 on target effect in cell model (FIG. 3A) of genomic DNA obtained from TZM-bl single cell clones: two controls (C1-2) and six Cas9/gRNA LTR 1+Gag D treated (E1-6). The presence of full length LTR −454/+43 (497 bp) was examined. Amplicons containing CRISPR-Cas9 specific InDel mutations at the LTR 1 target site in integrated HIV-1 LTR sequence are pointed by asterisks. Single asterisks indicate deletions, double asterisks insertions. FIG. 3B: Alignment of a representative Sanger sequencing results of HIV-1 LTR specific amplicons. The positions and nucleotide compositions of target for gRNA LTR1 is shown in green, PAM in red, sequence deletions in grey and sequence insertions in yellow, PCR primers in blue (SEQ ID NOS: 94-114). FIG. 3C: Representative Sanger sequencing tracing of LTR 1 region of HIV-1 LTRs obtained for each single cell clone. The positions and nucleotide compositions of target for gRNAs LTR1 is shown in green, PAM in red, sequence deletions in grey.

### DETAILED DESCRIPTION

Embodiments of the invention are directed to compositions that eliminate retrovirus genomes form an infected cell and the prevention of further infection by interfering with receptor expression or function that the virus uses to infect a cell. Compositions include the use of RNA-guided Clustered Regularly Interspace Short Palindromic Repeat (CRISPR)-Cas nuclease systems (Cas/gRNA) in single and multiplex configurations that target the retroviral genome as well as the genes encoding receptors used by the virus to infect a cell.

The CRISPR-Cas system includes a gene editing complex comprising a CRISPR-associated nuclease, e.g., Cas9, and a guide RNA complementary to a target sequence situated on a DNA strand, such as a target sequence in proviral DNA integrated into a mammalian genome, a receptor used by a virus to infect a cell, e.g. HIV and CCR5 receptor. The gene editing complex can cleave the DNA within the target sequence. This cleavage can in turn cause the introduction of various mutations into the proviral DNA, resulting in inactivation of HIV provirus. The mechanism by which such mutations inactivate the provirus can vary. For example, the mutation can affect proviral replication, and viral gene expression. The mutations may be located in regulatory sequences or structural gene sequences and result in defective production of HIV. The mutation can comprise a deletion. The size of the deletion can vary from a single nucleotide base pair to about 10,000 base pairs. In some embodiments, the deletion can include all or substantially all of the integrated retroviral DNA sequence. In some embodiments the deletion can include the entire integrated retroviral DNA sequence. The mutation can comprise an insertion, that is, the addition of one or more nucleotide base pairs to the pro-viral sequence. The size of the inserted sequence also may vary, for example from about one base pair to about 300 nucleotide base pairs. The mutation can comprise a point mutation, that is, the replacement of a single nucleotide with another nucleotide. Useful point mutations are those that have functional consequences, for example, mutations that result in the conversion of an amino acid codon into a termination codon or that result in the production of a nonfunctional protein.

In embodiments, the CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The invention is by no means limited to these variants, and also encompasses all Cas9 variants (Slaymaker, I. M. et al. Science. 2016 Jan. 1; 351(6268):84-8. doi: 10.1126/science.aad5227. Epub 2015 Dec. 1). The present invention also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems (Burstein, D. et al. New CRISPR-Cas systems from uncultivated microbes. Nature. 2017 Feb. 9; 542(7640):237-241. doi: 10.1038/nature21059. Epub 2016 Dec. 22).

Embodiments of the invention also include a new type of class 2 CRISPR-Cas system found in the genomes of two bacteria recovered from groundwater and sediment samples. This system includes Cas1, Cas2, Cas4 and an approximately ˜980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2017 supra).

Another new class 2 Cas protein is encoded in the genomes of certain candidate phyla radiation (CPR) bacteria. This approximately 1,200 amino acid Cas protein, termed CasY, appears to be part of a minimal CRISPR-Cas system that includes Cas1 and a CRISPR array. Most of the CRISPR arrays have unusually short spacers of 17-19 nt, but one system, which lacks Cas1 (CasY.5), has longer spacers (27-29 nt). Accordingly, in some embodiments of the invention, the CasY molecules comprise CasY.1, CasY.2, CasY.3, CasY.4, CasY.5, CasY.6, mutants, variants, analogs or fragments thereof.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas protein or fragment thereof. In other embodiments, the CRISPR/Cas-like protein can be derived from modified Cas proteins. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In some embodiments, the CRISPR-associated endonuclease can be a sequence from another species, for example, other bacterial species, bacteria genomes and archaea, or other prokaryotic microorganisms. Alternatively, the wild type Cas9, CasX, CasY.1, CasY.2, CasY.3, CasY.4, CasY.5, CasY.6, ARMAN 1, ARMAN 4, sequences can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in GENBANK accession numbers KM099231.1 GI:669193757; KM099232.1 GI:669193761; or KM099233.1 GI:669193765. Alternatively, the Cas9, CasX, CasY.1, CasY.2, CasY.3, CasY.4, CasY.5, CasY.6, ARMAN 1, ARMAN 4, sequences can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of GENBANK accession numbers KM099231.1 GI:669193757; KM099232.1 GI:669193761; or KM099233.1 GI:669193765, or Cas9 amino acid sequence of PX330 or PX260 (Addgene, Cambridge, Mass.).

The wild type Cas9, CasX, CasY.1, CasY.2, CasY.3, CasY.4, CasY.5, CasY.6, ARMAN 1, ARMAN 4, sequences can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The sequences of Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, can be modified to encode biologically active variants, and these variants can have or can include, for example, an amino acid sequence that differs from a wild type by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9, CasX, CasY.1, CasY.2, CasY.3, CasY.4, CasY.5, CasY.6, spCas, eSpCas, SpCas9, ARMAN 1, ARMAN 4 polypeptides. Examples of wild type Cas molecules are SEQ ID NOS: 1-20. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

In some embodiments, the isolated nucleic acids sequences can be encoded by the same construct with one or more isolated nucleic acids sequences directed toward a first and second retroviral target sequence, and one or more isolated nucleic acids sequences directed toward a one or more target sequences of one or more receptors that a virus uses to infect a cell, e.g. in the case of HIV, the receptor can be CCR5.

In some embodiments, the one or more isolated nucleic acids sequences are encoded by two or more constructs with one member directed toward a first retroviral target sequence, and the other member toward a second retroviral target sequence excises or eradicates the retroviral genome from an infected cell. Another construct is directed to a receptor that a virus uses to infect a cell, e.g. in the case of HIV, the receptor can be CCR5.

Accordingly, the invention features compositions for use in inactivating a proviral DNA integrated into a host cell, including an isolated nucleic acid sequence encoding a CRISPR-associated endonuclease and one or more isolated nucleic acid sequences encoding one or more gRNAs complementary to a target sequence in HIV or another retrovirus. A second isolated nucleic acid sequence encoding a CRISPR-associated endonuclease and one or more isolated nucleic acid sequences encoding one or more gRNAs complementary to a target sequence encoding a receptor used by a virus to infect a cell. The isolated nucleic acid can include one gRNA, two gRNAs, three gRNAs etc. Furthermore, the isolated nucleic acid can include one or more gRNAs complementary to target sequences in the retrovirus and a second isolated nucleic acid can include one or more gRNAs complementary to target sequences encoding receptors used by the virus to infect a cell. Alternatively each isolated nucleic acid can include at least one gRNA complementary to a target virus sequence and at least one a gRNA complementary to target sequences encoding receptors used by the virus to infect a cell. One of ordinary skill in the art would only be limited by their imagination with respect to the various combinations of gRNAs.

In some embodiments, a composition for preventing or treating a retroviral infection in vitro or in vivo comprises at least two isolated nucleic acid sequences encoding: a first Clustered Regularly Interspaced Short Palindromic Repeat (CRISPR)-associated endonuclease and at least one guide RNA (gRNA), the gRNA being complementary to a target sequence in the integrated retroviral DNA; a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo. In some embodiments, the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof. The endonucleases may be the same or may vary. For example, one endonuclease may be a Cas9, another endonuclease may be CasY.5 or ARMAN 4 and the like. Accordingly, the isolated nucleic acid sequence can encode any number and type of endonuclease.

In some embodiments, an isolated nucleic acid encoding for the endonuclease has a 60% sequence identity to any one or more of SEQ ID NOS: 1 to 20. In some embodiments, an isolated nucleic acid encoding for the endonuclease comprises any one or more of SEQ ID NOS: 1 to 20.

In some embodiments, at least one gRNA is complementary to a target sequence in the integrated retroviral DNA and at least one gRNA is complementary to a target sequence in a gene encoding for at least one receptor used by a retrovirus for attachment and/or infection of a cell. In another embodiment, two or more gRNAs are complementary to two or more different target sequences in the integrated retroviral DNA and two or more guide RNAs (gRNAs), are complementary to two or more target sequences in a gene encoding for at least one receptor used by a retrovirus for attachment and/or infection of a cell in vitro or in vivo. In some embodiments, the isolated nucleic acid encodes at least one gRNA complementary to a target sequence in the integrated retroviral DNA and at least a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell.

In some embodiments, the isolated nucleic acid encodes at least one gRNA complementary to a gene encoding at least one receptor used by a retrovirus for attachment and/or infection of a cell, and at least a first gRNA that is complementary to a first target sequence in the integrated retroviral DNA and at least a second gRNA that is complementary to a second target sequence in the integrated retroviral DNA. Accordingly, any number and combinations of gRNAs with different target sequences can be used to target desired target sequences.

In some embodiments, gRNA targets comprise one or more target sequences in an LTR region of an HIV proviral DNA and one or more targets in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

In some embodiments, gRNA targets comprise one or more target sequences in a gene encoding at least one receptor used by a retrovirus for attachment and/or infection of a cell and one or more targets in another gene associated with a viral infection; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

In some embodiments, a gRNA has at least about a 60% sequence identity to any one or more of SEQ ID NOS: 21-114 and to one or more target sequences of SEQ ID NOS: 115 and 116. In some embodiments, a gRNA comprises any one or more of SEQ ID NOS: 21-114 and to one or more target sequences of SEQ ID NOS: 115 and 116.

In some embodiments, a gRNA has a 60% sequence identity to any one or more of SEQ ID NOS: 21-24. In some embodiments, a gRNA comprises SEQ ID NOS: 21-24.

In certain embodiments, a composition for preventing or treating a retroviral infection in vitro or in vivo, the composition comprises at least two isolated nucleic acid sequences wherein the first isolated nucleic acid sequences encodes a first Clustered Regularly Interspaced Short Palindromic Repeat (CRISPR)-associated endonuclease and at least one guide RNA (gRNA), the gRNA being complementary to a target sequence in the integrated retroviral DNA; the second isolated nucleic acid sequences encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo.

In certain embodiments, the first isolated nucleic acid sequences encodes at least one gRNA, the gRNA being complementary to a target sequence in the integrated retroviral DNA and a second gRNA that is complementary to a second target sequence in the integrated retroviral DNA. In certain embodiments, the second isolated nucleic acid sequence encodes a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell. In certain embodiments, the first isolated nucleic acid sequence encodes a first gRNA, the gRNA being complementary to a target sequence in the integrated retroviral DNA and a second gRNA that is complementary to a target sequence in a gene encoding for at least one receptor used by a retrovirus for attachment and/or infection of a cell. In certain embodiments, the at least one receptor comprises CD4, CXCR4, CXCR5, variants or combinations thereof.

In certain embodiments, the first and second isolated nucleic acid sequences encode combinations of gRNAs having complementarity to one or more target sequences, the target sequences comprising retroviral DNA sequences, and sequences in one or more genes encoding for at least one receptor used by a retrovirus for attachment and/or infection of a cell.

In certain embodiments, the target sequence comprises one or more nucleic acid sequences in coding and non-coding nucleic acid sequences of the retrovirus genome.

In certain embodiments, the target sequences comprise one or more nucleic acid sequences in HIV comprising: long terminal repeat (LTR) nucleic acid sequences, nucleic acid sequences encoding structural proteins, non-structural proteins or combinations thereof.

In certain embodiments, the sequences encoding structural proteins comprise nucleic acid sequences encoding: Gag, Gag-Pol precursor, Pro (protease), Reverse Transcriptase (RT), integrase (In), Env or combinations thereof.

In certain embodiments, the sequences encoding non-structural proteins comprise nucleic acid sequences encoding: regulatory proteins, accessory proteins or combinations thereof.

In certain embodiments, the regulatory proteins comprise: Tat, Rev or combinations thereof.

In certain embodiments, the accessory proteins comprise Nef, Vpr, Vpu, Vif or combinations thereof.

In certain embodiments, the gRNA target sequences comprise one or more target sequences in an LTR region of an HIV proviral DNA and one or more target sequences in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

In certain embodiments, a gRNA has a 60% sequence identity to any one or more of a gRNA has a 60% sequence identity to any one or more of SEQ ID NOS: 21-114 and to one or more target sequences of SEQ ID NOS: 115 and 116.

In certain embodiments, a gRNA comprises SEQ ID NOS: 21-114 and to one or more target sequences of SEQ ID NOS: 115 and 116.

In certain embodiments, a gRNA has a 60% sequence identity to any one or more of SEQ ID NOS: 21-24.

In certain embodiments, a gRNA comprises SEQ ID NOS: 21-24.

In certain embodiments, the first Clustered Regularly Interspaced Short Palindromic Repeat (CRISPR)-associated endonuclease and at least one guide RNA (gRNA) at least one gRNA comprising SEQ ID NOS: 25-116; wherein the second Clustered Regularly Interspaced Short Palindromic Repeat (CRISPR)-associated endonuclease and at least one guide RNA (gRNA) comprising SEQ ID NOS: 21-24.

In certain embodiments, the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof.

In certain embodiments, the nucleic acid encoding for the endonuclease has at least a 60% sequence identity to any one or more of SEQ ID NOS: 1 to 20.

In certain embodiments, the nucleic acid encoding for the endonuclease comprises any one or more of SEQ ID NOS: 1 to 20.

In another embodiment, an isolated nucleic acid sequence encoding a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo. In some embodiments, the isolated nucleic acid sequence further comprises two or more gRNAs complementary to a target sequence in the integrated retroviral DNA; and/or two or more gRNAs complementary to a target sequence in a gene encoding for at least one receptor used by a retrovirus for attachment and/or infection of a cell in vitro or in vivo. In some embodiments, the isolated nucleic acid sequence further comprises a combination of one or more gRNAs complementary to a target sequence in the integrated retroviral DNA; and/or one or more gRNAs complementary to a target sequence in a gene encoding for at least one receptor used by a retrovirus for attachment and/or infection of a cell in vitro or in vivo. In some embodiments, the isolated nucleic acid sequence further comprises two or more gRNAs complementary to a target sequence in the integrated retroviral DNA; and/or two or more gRNAs complementary to a target sequence in a gene encoding for at least one receptor used by a retrovirus for attachment and/or infection of a cell in vitro or in vivo. In some embodiments, a gRNA has a 60% sequence identity to any one or more of SEQ ID NOS: 21-114 and to one or more target sequences of SEQ ID NOS: 115 and 116. In other embodiments, a gRNA comprises SEQ ID NOS: 21-114 and to one or more target sequences of SEQ ID NOS: 115 and 116. In some embodiments, one or more endonucleases comprise Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof. Accordingly, any one or combinations thereof of endonucleases can be combined with one or more gRNAs. In some embodiments, a nucleic acid encoding for the endoncuclease has a 60% sequence identity to any one or more of SEQ ID NOS: 1 to 20 and/or the endoncuclease comprises any one or more of SEQ ID NOS: 1 to 20, or any combinations thereof.

Guide RNA Sequences: The compositions and methods of the present invention may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (hiv.lanl.gov). The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K, or group (for example, but not limited to any of the following Groups, N, O and P) of HIV.

A gRNA includes a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). In the present invention, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from U6 or H1-promoted RNA expression vector.

In the compositions of the present invention, each gRNA includes a sequence that is complementary to a target sequence in a retrovirus. The exemplary target retrovirus is HIV, but the compositions of the present invention are also useful for targeting other retroviruses, such as HIV-2 and simian immunodeficiency virus (SIV)-1. The guide RNA can be a sequence complimentary to a coding or a non-coding sequence (i.e., a target sequence). For example, the guide RNA can be a sequence that is complementary to a HIV long terminal repeat (LTR) region.

Some of the exemplary gRNAs of the present invention are complimentary to target sequences in the long terminal repeat (LTR) regions of HIV. The LTRs are subdivided into U3, R and U5 regions. LTRs contain all of the required signals for gene expression, and are involved in the integration of a provirus into the genome of a host cell. For example, the basal or core promoter, a core enhancer and a modulatory region is found within U3 while the transactivation response element is found within R. In HIV-1, the U5 region includes several sub-regions, for example, TAR or trans-acting responsive element, which is involved in transcriptional activation; Poly A, which is involved in dimerization and genome packaging; PBS or primer binding site; Psi or the packaging signal; DIS or dimer initiation site. Accordingly, in some embodiments, gRNA targets comprise one or more target sequences in an LTR region of an HIV proviral DNA and one or more targets in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof. Furthermore, gRNA targets directed to one or more sequences encoding a receptor for viral entry, e.g. CCR5.

Receptors for viral entry include the CD4 receptor to which the HIV gp120 attaches. The CD4 receptor is found on CD4 T-cells and macrophages. Additionally, after gp120 successfully attaches to the CD4 cell, it can change shape to avoid recognition by the CD4 cell's neutralising antibodies, a process known as conformational masking. The conformational change in gp120 allows it to bind to a second receptor on the CD4 cell surface.

The second docking area on the CD4 cell surface is a chemokine receptor and there are two possibilities, CCR5 or CXCR4. The viral preference for using one co-receptor versus another is called ‘viral tropism’. Chemokine receptor 5 (CCR5), is used by macrophage-tropic (M-tropic) HIV to bind to a cell. About 90% of all HIV infections involve the M-tropic HIV strain. CXCR4, also called fusin, is a glycoprotein-linked chemokine receptor used by T-tropic HIV (ones that preferentially infect CD4 T-cells) to attach to the host cell.

Once the HIV envelope has attached to the CD4 molecule and is bound to a chemokine co-receptor, the HIV envelope utilizes a structural change in the gp41 envelope protein to fuse with the cell membrane. The HIV virion is then able to penetrate the CD4 membrane. Once within a cell, virus is safe from attack by antibodies, but vulnerable to attack by CD8 cells (cytotoxic T-lymphocytes or CTLs).

CCR5: Macrophage (M-tropic) strains of HIV-1 use the β-chemokine receptor CCR5 for binding and are able to infect macrophages, dendritic cells, and CD4 T-cells. Almost all HIV-1 isolates are successfully transmitted using the CCR5 co-receptor. M-tropic HIV replicates in peripheral blood lymphocytes and does not form syncytia. Syncytia are ‘giant cells’, multicellular clumps that have been formed by fusing with other cells. Non-syncytia-inducing (NSI) strains of virus are considered less virulent than those that do form syncytia.

Some people have a 32-base pair deletion (delta 32) in the gene that encodes the CCR5 receptor. If they receive this deletion from both parents, they are said to be homozygous for CCR5-delta32. This deletion is highly protective because the receptor is faulty and HIV cannot use it to enter the cell.

There have been a few cases in which someone homozygous for the deletion was infected with dual-tropic HIV and suffered rapid depletion of CD4 T-cells. This is the exception. Ordinarily, it is a great advantage to have this deletion. If someone inherits the deletion from just one parent, they are said to be heterozygous for CCR5 and this can slow HIV progression. The prevalence of 32-base pair deletion is estimated to be as high as 10 to 15% in Caucasians, but only around 2% in African Americans and almost non-existent in native Africans and East Asians.

Other mutations in CCR5 that effect disease progression have also been identified, including some that might play a protective role in HIV acquisition or progression in non-Caucasian people. Slower disease progression is also associated with high levels of the CCR5 59353-C polymorphism in the promoter DNA that controls the amount of CCR5 that cells produce.

Variations also occur in the amount of chemokines in people's blood. Chemokines compete with HIV for chemokine receptors, preventing HIV from using the receptors and reducing the susceptibility of cells to infection. Unusually high levels of the CCR5-using chemokines RANTES, MIP-1 alpha, and MIP-1 beta are seen in long-term non-progressors, as well as in exposed seronegative individuals (people with repeated exposure to the virus through unprotected sex who do not become infected).

The data herein show the functionality of the CCR5-HIV dual targeting vector. This includes evidence that the CCR5 gRNAs cleave the CCR5 receptor gene target and result in reduced HIV replication in TZM-b 1 cells, and evidence that the HIV-1 LTR1 gRNAs cleave their target HIV sequences.

CXCR4: CXCR4, also known as fusin or X4, is the receptor used by T-tropic strains of HIV. T-tropic HIV attaches first to the CD4 receptor and then to the α-chemokine receptor CXCR4. T-tropic HIV can be syncytium-inducing (SI) and the presence of SI-inducing variants of HIV has been correlated with rapid disease progression in HIV-positive individuals.

CXCR4-tropic HIV strains tend to emerge in the body during the course of HIV infection. People whose virus uses the CXCR4 co-receptor tend to have higher viral loads and much lower CD4 cell counts. Studies suggest that the presence of the CXCR4-using strain does not affect the outcome of antiretroviral therapy.

As with CXCR5, a proportion of the population has a genetic mutation that impairs the efficiency or ability of T-tropic virus to attach. Around 1% of Caucasians do not produce this co-receptor, reducing their susceptibility to CXCR4-tropic strains of HIV.

Dual and mixed-tropic HIV: M-tropic and T-tropic strains of HIV coexist in the body. At some point in infection, gp120 is able to attach to either CCR5 or CXCR4. This is called dual tropic virus or R5X4 HIV. Virus that can utilise the CXCR4 receptor on both macrophages and T-cells is also termed dual-tropic X4 HIV Mixed tropism results when an individual has two virus populations; one using CCR5 and the other CXCR4 to bind to the CD4 T-cell.

Generally, CCR5 is expressed by memory CD4 T-cells and CXCR4 is expressed by naive CD4 T-cells. In a healthy immune system, memory cells divide at much higher rates (approximately tenfold) than naive CD4 T-cells. CXCR4-tropic virus is probably disadvantaged during early infection when there is a great abundance of memory CD4 T-cells present. With disease progression, naive cell division is more approximate to that of memory cells and there tends to be a shift in tropism from CCR5 to CXCR4. This would imply that the emergence of CXCR4-using virus is both a cause and a consequence of immunodeficiency.

Accordingly, in certain embodiments, the guide RNAs are complementary to one or more target sequences to one or more receptors to which an HIV virus binds, comprising: wherein the at least one receptor comprises CD4, CXCR4, CXCR5, variants or combinations thereof.

Some of the exemplary gRNAs of the present invention target sequences in the coding and non-coding protein coding genome of HIV. gRNAs complementary to LTR target sequences include LTR 1, LTR 2, LTR 3, LTR A, LTR B, LTR B′, LTR C, LTR D, LTR E, LTR F, LTR G, LTR H, LTR I, LTR J, LTR K, LTR L, LTR M, LTR N, LTR O, LTR P, LTR Q, LTR R, LTR S, AND LTR T. gRNAs complementary to Gag target sequences include Gag A, Gag B, Gag C, and Gag D. gRNAs complementary to pol target sequences include Pol A and Pol B. Accordingly, the compositions of the present invention include these exemplary gRNAs, but are not limited to them, and can include gRNAs complimentary to any suitable target site in the protein coding genes of HIV, including but not limited to those encoding the envelope protein env, the structural protein tat, and the accessory proteins vif, willef (negative factor) vpu (Virus protein U) and tev.

Guide RNA sequences according to the present invention can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR 3) and Neiseria meningitidis requires 5′-NNNNGATT). The specific sequence of the guide RNA may vary, but, regardless of the sequence, useful guide RNA sequences will be those that minimize off-target effects while achieving high efficiency and complete ablation of the genomically integrated HIV-1 provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV-1 LTR promoter (potentially conserved in the host genome); and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

Combinations of gRNAs are especially effective when expressed in multiplex fashion, that is, simultaneously in the same cell. In many cases, the combinations produce excision of the HIV provirus extending between the target sites. The excisions are attributable to deletions of sequences between the cleavages induced by the endonuclease at each of the multiple target sites. These combinations pairs of gRNAs, with one member being complementary to a target site in an LTR of the retrovirus, and the other member being complementary to a gRNA complementary to a target site in a structural gene of the retrovirus. Exemplary effective combinations include Gag D combined with one of LTR 1, LTR 2, LTR 3, LTR A, LTR B, LTR C, LTR D, LTR E, LTR F, LTR G; LTR H, LTR I, LTR J, LTR K, LTR L, LTR M; LTR N, LTR O, LTR P, LTR Q, LTR R, LTR S, or LTR T. Exemplary effective combinations also include LTR 3 combined with one of LTR-1, Gag A; Gag B; Gag C, Gag D, Pol A, or Pol B. see, for example, Table 1.

The compositions of present invention are not limited to these combinations, but include any suitable combination of gRNAS complimentary to two or more different target sites in the retroviral provirus.

Accordingly, the present invention also includes a method of inactivating a proviral DNA integrated into the genome of a host cell latently infected with a retrovirus, the method including the steps of treating the host cell with a composition comprising a CRISPR-associated endonuclease, and at least one gRNA complementary to a target site in the proviral DNA; at least one gRNA complementary to a target site of one or more genes encoding receptors used by a virus for infecting a cell; expressing a gene editing complex including the CRISPR-associated endonuclease and the at least one gRNA; and inactivating the proviral DNA and the receptor. In another preferred embodiment, the step of treating the host cell includes treatment with at least two gRNAs, wherein each of the at least two gRNAs are complementary to a different target nucleic acid sequence in the proviral DNA and one or more gRNAs complementary to a different target nucleic acid sequence in one or more nucleic acid sequences encoding for a receptor that can be used by a virus to infect a cell. Especially preferred are combinations of at least two gRNAs, including compositions wherein at least one gRNA is complementary to a target site in an LTR of the retrovirus, and at least one gRNA is complementary to a target site in a structural gene of the retrovirus. An example is as follows:

```
H (HIV-1) gRNAs:
  (SEQ ID NO: 21)
LTR1 5′-GCAGAACTACACACCAGGGCC-3′;
  (SEQ ID NO: 22)
gagD 5′-GGATAGATGTAAAAGACACCA-3′.
```

With respect to a receptor that a virus uses to infect a cell comprises:

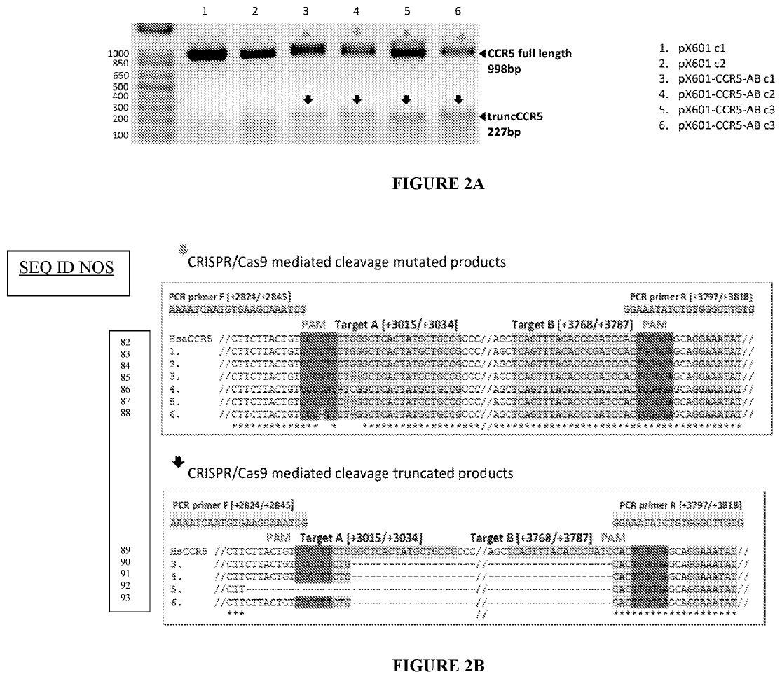

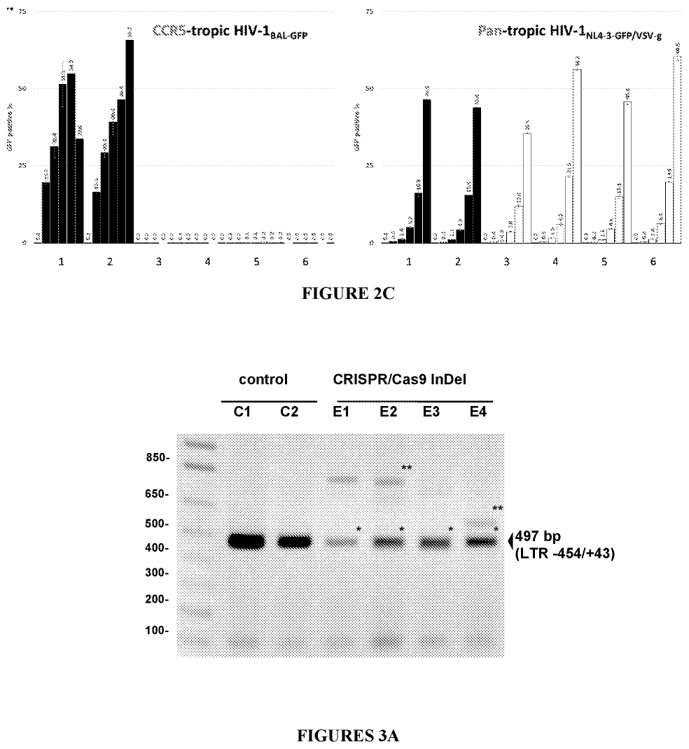

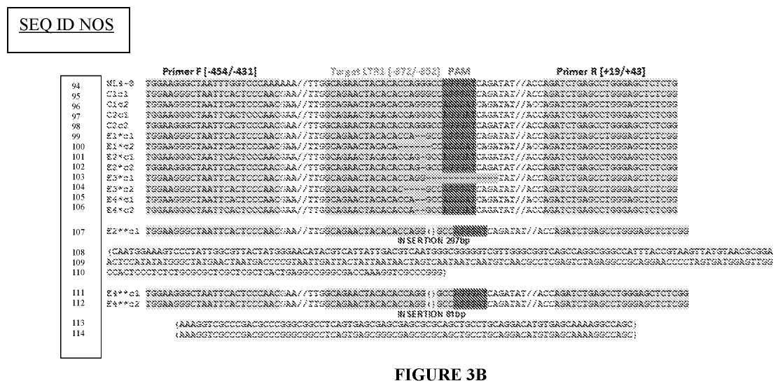

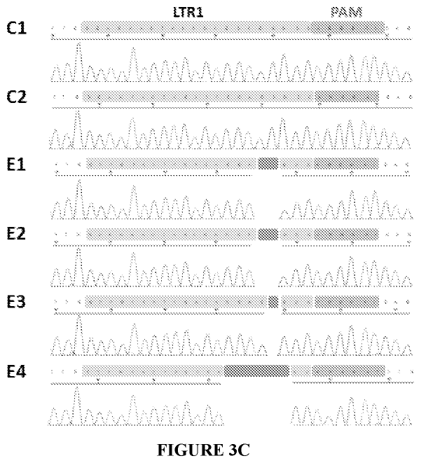

```
C (HsCCR5) gRNAs:
  (SEQ ID NO: 23)
CCR5 A 5′-GCGGCAGCATAGTGAGCCCAG-3′;
  (SEQ ID NO: 24)
CCR5 B 5′-TCAGTTTACACCCGATCCAC-3′;
SEQ ID NOS: 82-93 (FIG. 2B).
```

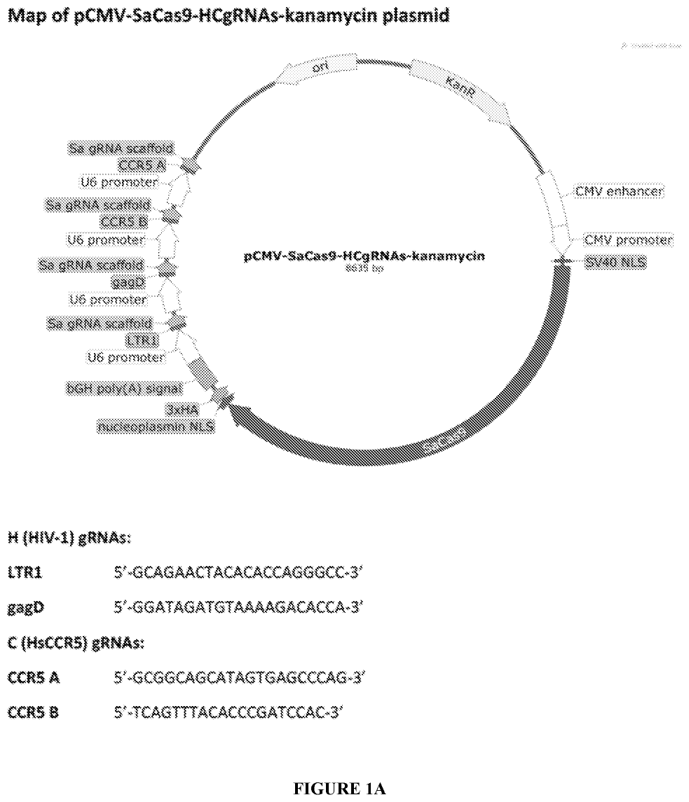

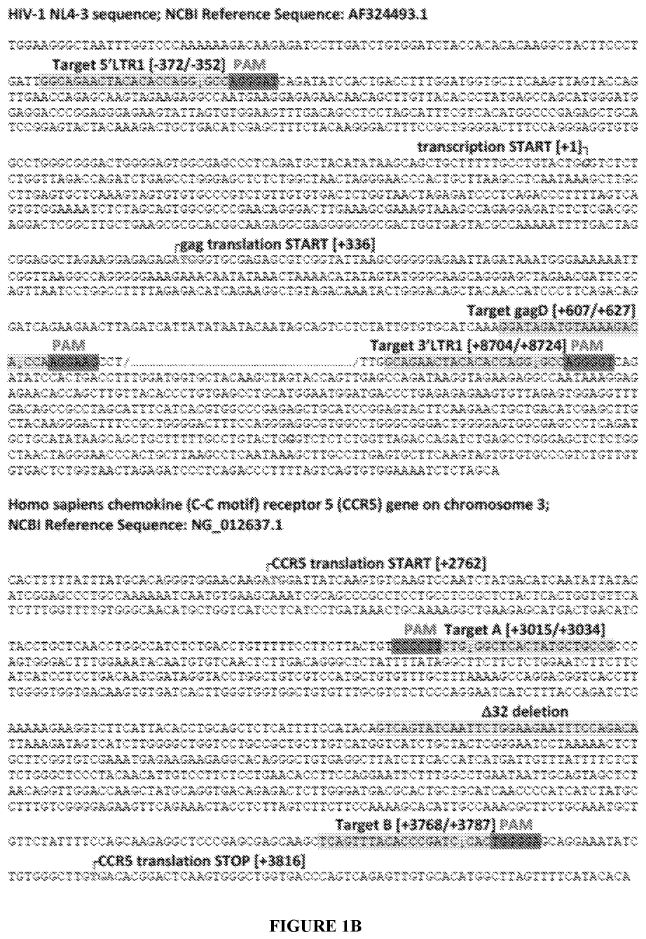

In certain embodiments, a gRNA is complementary to one or more target sequences of human CCR5 gene (NCBI Reference Sequence NG_012637.1; FIG. 1B).

In certain embodiments, a gRNA is complementary to one or more target sequences of SEQ ID NOS: 21-114 and to one or more target sequences of SEQ ID NOS: 115 and 116

These are only meant as examples and are not to be construed as limiting the invention in any way. When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

The gRNA sequences according to the present invention can be complementary to either the sense or anti-sense strands of the target sequences. They can include additional 5′ and/or 3′ sequences that may or may not be complementary to a target sequence. They can have less than 100% complementarity to a target sequence, for example 75% complementarity. The gRNA sequences can be employed as a combination of one or more different sequences, e.g., a multiplex configuration. Multiplex configurations can include combinations of two, three, four, five, six, seven, eight, nine, ten, or more different guide RNAs.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH,—N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluri dine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶,N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1,2′-O-dimethyl inosine; 2′-O-methyl guanosine; 1-methyl guanosine; N²-methyl guanosine; N²,N²-dimethyl guanosine; N²,2′-O-dimethyl guanosine; N²,N²,2′-O-trimethyl guanosine; 2′-O-ribosyl guanosine (phosphate); 7-methyl guanosine; N²,7-dimethyl guanosine; N²,N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

The present invention also includes a pharmaceutical composition for the inactivation of integrated proviral HIV-1 DNA in a mammalian subject and the prevention of further infection by targeting receptors used by a virus to infect a cell. The composition includes an isolated nucleic acid sequence encoding a Cas endonuclease, e.g. Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof; at least one isolated nucleic acid sequence encoding at least one gRNA complementary to a target sequence in a proviral HIV DNA; and at least one isolated nucleic acid sequence encoding at least one gRNA complementary to a target sequence in a receptor used by a virus to infect a cell. In some embodiments, the isolated nucleic acid sequences are included in at least one expression vector. In some embodiments, the pharmaceutical composition includes a first gRNA and a second gRNA, with the first gRNA targeting a site in the HIV LTR and the second gRNA targeting a site in an HIV structural gene; and, a third gRNA and/or a fourth gRNA wherein the third gRNA is complementary to a target sequence in a receptor used by a virus to infect a cell. The fourth gRNA can be targeted to a different receptor or to a second target site of a nucleic acid encoding the receptor.

Exemplary expression vectors for inclusion in the pharmaceutical composition include plasmid vectors and lentiviral vectors, but the present invention is not limited to these vectors. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

The method represents a solution to the problem of integrated provirus, a solution which is essential to the treatment and prevention of AIDS and other retroviral diseases. During the acute phase of HIV infection, the HIV viral particles are attracted to and enter cells expressing the appropriate CD4 receptor molecules. Once the virus has entered the host cell, the HIV encoded reverse transcriptase generates a proviral DNA copy of the HIV RNA and the proviral DNA becomes integrated into the host cell genomic DNA. It is this HIV provirus that is replicated by the host cell, resulting in the release of new HIV virions which can then infect other cells.

The primary HIV infection subsides within a few weeks to a few months, and is typically followed by a long clinical “latent” period which may last for up to 10 years. During this latent period, there can be no clinical symptoms or detectable viral replication in peripheral blood mononuclear cells and little or no culturable virus in peripheral blood. However, the HIV virus continues to reproduce at very low levels. In subjects who have treated with anti-retroviral therapies, this latent period may extend for several decades or more. Anti-retroviral therapy does not suppress low levels of viral genome expression, nor does it efficiently target latently infected cells such as resting memory T cells, brain macrophages, microglia, astrocytes and gut associated lymphoid cells. Because the compositions of the present invention can inactivate or excise HIV-provirus, and can prevent the infection of cells by preventing expression or function the virus receptor, the methods of treatment employing the compositions constitute a new avenue of attack against HIV-1 infection

The compositions of the present invention, when stably expressed in potential host cells, reduce or prevent new infection by HIV. Accordingly, the present invention also provides a method of treatment to reduce the risk of HIV infection in a mammalian subject at risk for infection. The method includes the steps of determining that a mammalian subject is at risk of HIV infection, administering an effective amount of the previously described pharmaceutical composition, and reducing the risk of HIV infection in the mammalian subject. Preferably, the pharmaceutical composition includes a vector that provides stable and/or inducible expression of at least one of the previously enumerated.

Pharmaceutical compositions according to the present invention can be prepared in a variety of ways known to one of ordinary skill in the art. For example, the nucleic acids and vectors described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein, in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are common latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating a nucleic acid encoding Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, and at least one gRNA sequence complementary to a target HIV and/or to a receptor target sequence, such as CCR5; or it can include a vector encoding these components. Alternatively, the compositions can be formulated as a nanoparticle encapsulating the CRISPR-associated endonuclease the polypeptides encoded by one or more of the nucleic acid compositions of the present invention.

In methods of treatment of HIV-1 infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R or U5 region, or pol, gag, or env genes, region and then design or select one or more gRNAs to be precisely complementary to the patient's sequences. The novel gRNAs provided by the present invention greatly enhance the chances of formulating an effective treatment. The gRNAs targeted to nucleic acid sequences encoding a receptor used by a virus to infect a cell would prevent further infection.

In methods of reducing the risk of HIV infection, a subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

Combination Therapies

In certain embodiments, the gene-editing compositions embodied herein are administered to a patient in combination with one or more other anti-viral agents or therapeutics. Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

In certain embodiments, the gene-editing compositions embodied herein are administered with one or more compositions comprising a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, a composition comprises a therapeutically effective amount of at least one NNRTI or a combination of NNRTI's, analogs, variants or combinations thereof. In certain embodiments, the NNRTI is rilpivirine.In certain embodiments, an NRTI comprises: lamivudine, zidovudine, emtricitabine, abacavir, zalcitabine, dideoxycytidine, azidothymidine, tenofovir disoproxil fumarate, didanosine (ddl EC), dideoxyinosine, stavudine, abacavir sulfate or combinations thereof. In certain embodiments, the composition comprises a therapeutically effective amount of at least one or a combination of NRTI's, analogs, variants or combinations thereof.

Kit

The present invention also includes a kit including an isolated nucleic acid sequence encoding a CRISPR-associated endonuclease, for example, a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4 endonucleases, and at least one isolated nucleic acid sequence encoding a gRNA complementary to a target sequence in an HIV provirus and at least one isolated nucleic acid sequence encoding a gRNA complementary to a target sequence in a gene or nucleic acid sequence encoding a receptor that is used by a virus to infect a cell. Alternatively, at least one of the isolated nucleic acid sequences can be encoded in a vector, such as an expression vector. Possible uses of the kit include the treatment or prophylaxis of HIV infection. Preferably, the kit includes instructions for use, syringes, delivery devices, buffers sterile containers and diluents, or other reagents for required for treatment or prophylaxis. The kit can also include a suitable stabilizer, a carrier molecule, a flavoring, or the like, as appropriate for the intended use.

```
CasY.1 Candidatus katanobacteria amino acid sequence 1125 aa (SEQ ID
NO: 1):
MRKKLFKGYILHNKRLVYTGKAAIRSIKYPLVAPNKTALNNLSEKIIY
DYEHLFGPLNVASYARNSNRYSLVDFWIDSLRAGVIWQSKSTSLIDLISKLEGSKSPS
EKIFEQIDFELKNKLDKEQFKDIILLNTGIRSSSNVRSLRGRFLKCFKEEFRDTEEVIAC
VDKWSKDLIVEGKSILVSKQFLYWEEEFGIKIFPHFKDNHDLPKLTFFVEPSLEFSPHL
PLANCLERLKKFDISRESLLGLDNNFSAFSNYFNELFNLLSRGEIKKIVTAVLAVSKS
WENEPELEKRLHFLSEKAKLLGYPKLTSSWADYRMIIGGKIKSWHSNYTEQLIKVRE
DLKKHQIALDKLQEDLKKVVDSSLREQIEAQREALLPLLDTMLKEKDFSDDLELYRF
ILSDFKSLLNGSYQRYIQTEEERKEDRDVTKKYKDLYSNLRNIPRFFGESKKEQFNKFI
NKSLPTIDVGLKILEDIRNALETVSVRKPPSITEEYVTKQLEKLSRKYKINAFNSNRFK
QITEQVLRKYNNGELPKISEVFYRYPRESHVAIRILPVKISNPRKDISYLLDKYQISPD
WKNSNPGEVVDLIEIYKLTLGWLLSCNKDFSMDFSSYDLKLFPEAASLIKNFGSCLSG
YYLSKMIFNCITSEIKGMITLYTRDKFVVRYVTQMIGSNQKFPLLCLVGEKQTKNFSR
NWGVLIEEKGDLGEEKNQEKCLIFKDKTDFAKAKEVEIFKNNIWRIRTSKYQIQFLNR
LFKKTKEWDLMNLVLSEPSLVLEEEWGVSWDKDKLLPLLKKEKSCEERLYYSLPLN
LVPATDYKEQSAEIEQRNTYLGLDVGEFGVAYAVVRIVRDRIELLSWGFLKDPALRK
IRERVQDMKKKQVMAVFSSSSTAVARVREMAIHSLRNQIHSIALAYKAKIIYEISISNF
ETGGNRMAKIYRSIKVSDVYRESGADTLVSEMIWGKKNKQMGNHISSYATSYTCCN
CARTPFELVIDNDKEYEKGGDEFIFNVGDEKKVRGFLQKSLLGKTIKGKEVLKSIKEY
ARPPIREVLLEGEDVEQLLKRRGNSYIYRCPFCGYKTDADIQAALNIACRGYISDNAK
DAVKEGERKLDYILEVRKLWEKNGAVLRSAKFL
CasY.1 Candidatus katanobacteria nucleic acid sequence (SEQ ID NO: 2):
                at gcgcaaaaaa ttgtttaagg gttacatttt acataataag aggcttgtat atacaggtaa agctgcaata
cgttctatta aatatccatt agtcgctcca aataaaacag ccttaaacaa tttatcagaa aagataattt atgattatga gcatttattc
ggacctttaa atgtggctag ctatgcaaga aattcaaaca ggtacagcct tgtggatttt tggatagata gcttgcgagc
aggtgtaatt tggcaaagca aaagtacttc gctaattgat ttgataagta agctagaagg atctaaatcc ccatcagaaa
agatatttga acaaatagat tttgagctaa aaaataagtt ggataaagag caattcaaag atattattct tcttaataca ggaattcgtt
ctagcagtaa tgttcgcagt ttgagggggc gctttctaaa gtgttttaaa gaggaattta gagataccga agaggttatc
gcctgtgtag ataaatggag caaggacctt atcgtagagg gtaaaagtat actagtgagt aaacagtttc tttattggga
agaagagttt ggtattaaaa tttttcctca ttttaaagat aatcacgatt taccaaaact aacttttttt gtggagcctt ccttggaatt
tagtccgcac ctccctttag ccaactgtct tgagcgtttg aaaaaattcg atatttcgcg tgaaagtttg ctcgggttag acaataattt
ttcggccttt tctaattatt tcaatgagct ttttaactta ttgtccaggg gggagattaa aaagattgta acagctgtcc ttgctgtttc
taaatcgtgg gagaatgagc cagaattgga aaagcgctta cattttttga gtgagaaggc aaagttatta gggtacccta
agcttacttc ttcgtgggcg gattatagaa tgattattgg cggaaaaatt aaatcttggc attctaacta taccgaacaa
ttaataaaag ttagagagga cttaaagaaa catcaaatcg cccttgataa attacaggaa gatttaaaaa aagtagtaga
tagctcttta agagaacaaa tagaagctca acgagaagct ttgcttcctt tgcttgatac catgttaaaa gaaaaagatt
tttccgatga tttagagctt tacagattta tcttgtcaga ttttaagagt ttgttaaatg ggtcttatca aagatatatt caaacagaag
aggagagaaa ggaggacaga gatgttacca aaaaatataa agatttatat agtaatttgc gcaacatacc tagatttttt
ggggaaagta aaaaggaaca attcaataaa tttataaata aatctctccc gaccatagat gttggtttaa aaatacttga
ggatattcgt aatgctctag aaactgtaag tgttcgcaaa cccccttcaa taacagaaga gtatgtaaca aagcaacttg
agaagttaag tagaaagtac aaaattaacg cctttaattc aaacagattt aaacaaataa ctgaacaggt gctcagaaaa
tataataacg gagaactacc aaagatctcg gaggtttttt atagataccc gagagaatct catgtggcta taagaatatt
acctgttaaa ataagcaatc caagaaagga tatatcttat cttctcgaca aatatcaaat tagccccgac tggaaaaaca
gtaacccagg agaagttgta gatttgatag agatatataa attgacattg ggttggctct tgagttgtaa caaggatttt
tcgatggatt tttcatcgta tgacttgaaa ctcttcccag aagccgcttc cctcataaaa aattttggct cttgcttgag tggttactat
ttaagcaaaa tgatatttaa ttgcataacc agtgaaataa aggggatgat tactttatat actagagaca agtttgttgt tagatatgtt
acacaaatga taggtagcaa tcagaaattt cctttgttat gtttggtggg agagaaacag actaaaaact tttctcgcaa
ctggggtgta ttgatagaag agaagggaga tttgggggag gaaaaaaacc aggaaaaatg tttgatattt aaggataaaa
cagattttgc taaagctaaa gaagtagaaa tttttaaaaa taatatttgg cgtatcagaa cctctaagta ccaaatccaa tattgaata
ggctttttaa gaaaaccaaa gaatgggatt taatgaatct tgtattgagc gagcctagct tagtattgga ggaggaatgg
ggtgtttcgt gggataaaga taaactttta cctttactga agaaagaaaa atcttgcgaa gaaagattat attactcact
tccccttaac ttggtgcctg ccacagatta taaggagcaa tctgcagaaa tagagcaaag gaatacatat ttgggtttgg
atgttggaga atttggtgtt gcctatgcag tggtaagaat agtaagggac agaatagagc ttctgtcctg gggattcctt
aaggacccag ctcttcgaaa aataagagag cgtgtacagg atatgaagaa aaagcaggta atggcagtat tttctagctc
ttccacagct gtcgcgcgag tacgagaaat ggctatacac tctttaagaa atcaaattca tagcattgct ttggcgtata
aagcaaagat aatttatgag atatctataa gcaattttga gacaggtggt aatagaatgg ctaaaatata ccgatctata
aaggtttcag atgtttatag ggagagtggt gcggataccc tagtttcaga gatgatctgg ggcaaaaaga ataagcaaat
gggaaaccat atatcttcct atgcgacaag ttacacttgt tgcaattgtg caagaacccc ttttgaactt gttatagata
atgacaagga atatgaaaag ggaggcgacg aatttatttt taatgttggc gatgaaaaga aggtaagggg gtttttacaa
aagagtctgt taggaaaaac aattaaaggg aaggaagtgt tgaagtctat aaaagagtac gcaaggccgc ctataaggga
agtcttgctt gaaggagaag atgtagagca gttgttgaag aggagaggaa atagctatat ttatagatgc cctttttgtg
gatataaaac tgatgcggat attcaagcgg cgttgaatat agcttgtagg ggatatattt cggataacgc aaaggatgct
gtgaaggaag gagaaagaaa attagattac attttggaag ttagaaaatt gtgggagaag aatggagctg attgagaag
cgccaaattt ttatagtt
CasY.2 Candidatus vogelbacteria amino acid sequence 1226 aa (SEQ ID
NO: 3):
MQKVRKTLSEVHKNPYGTKVRNAKTGYSLQIERLSYTGKEGMRSFKI
PLENKNKEVFDEFVKKIRNDYISQVGLLNLSDWYEHYQEKQEHYSLADFWLDSLRA
GVIFAHKETEIKNLISKIRGDKSIVDKFNASIKKKHADLYALVDIKALYDFLTSDARRG
LKTEEEFFNSKRNTLFPKFRKKDNKAVDLWVKKFIGLDNKDKLNFTKKFIGFDPNPQ
IKYDHTFFFHQDINFDLERITTPKELISTYKKFLGKNKDLYGSDETTEDQLKMVLGFH
NNHGAFSKYFNASLEAFRGRDNSLVEQIINNSPYWNSHRKELEKRIIFLQVQSKKIKE
TELGKPHEYLASFGGKFESWVSNYLRQEEEVKRQLFGYEENKKGQKKFIVGNKQEL
DKIIRGTDEYEIKAISKETIGLTQKCLKLLEQLKDSVDDYTLSLYRQLIVELRIRLNVEF
QETYPELIGKSEKDKEKDAKNKRADKRYPQIFKDIKLIPNFLGETKQMVYKKFIRSAD
ILYEGINFIDQIDKQITQNLLPCFKNDKERIEFTEKQFETLRRKYYLMNSSRFHHVIEGII
NNRKLIEMKKRENSELKTFSDSKFVLSKLFLKKGKKYENEVYYTFYINPKARDQRRI
KIVLDINGNNSVGILQDLVQKLKPKWDDIIKKNDMGELIDAIEIEKVRLGILIALYCEH
KFKIKKELLSLDLFASAYQYLELEDDPEELSGTNLGRFLQSLVCSEIKGAINKISRTEYI
ERYTVQPMNTEKNYPLLINKEGKATWHIAAKDDLSKKKGGGTVAMNQKIGKNFFG
KQDYKTVFMLQDKRFDLLTSKYHLQFLSKTLDTGGGSWWKNKNIDLNLSSYSFIFE
QKVKVEWDLTNLDHPIKIKPSENSDDRRLFVSIPFVIKPKQTKRKDLQTRVNYMGIDI
GEYGLAWTIINIDLKNKKINKISKQGFIYEPLTHKVRDYVATIKDNQVRGTFGMPDTK
LARLRENAITSLRNQVHDIAMRYDAKPVYEFEISNFETGSNKVKVIYDSVKRADIGR
GQNNTEADNTEVNLVWGKTSKQFGSQIGAYATSYICSFCGYSPYYEFENSKSGDEEG
ARDNLYQMKKLSRPSLEDFLQGNPVYKTFRDFDKYKNDQRLQKTGDKDGEWKTHR
GNTAIYACQKCRHISDADIQASYWIALKQVVRDFYKDKEMDGDLIQGDNKDKRKV
NELNRLIGVHKDVPIINKNLITSLDINLL
CasY.2 Candidatus vogelbacteria nucleic acid sequence (SEQ ID NO: 4):
                  a tggtattagg ttttcataat aatcacggcg ctttttctaa gtatttcaac gcgagcttgg aagcttttag
ggggagagac aactccttgg ttgaacaaat aattaataat tctccttact ggaatagcca tcggaaagaa ttggaaaaga
gaatcatttt tttgcaagtt cagtctaaaa aaataaaaga gaccgaactg ggaaagcctc acgagtatct tgcgagtttt
ggcgggaagt ttgaatcttg ggtttcaaac tatttacgtc aggaagaaga ggtcaaacgt caactttttg gttatgagga
gaataaaaaa ggccagaaaa aatttatcgt gggcaacaaa caagagctag ataaaatcat cagagggaca gatgagtatg
agattaaagc gatttctaag gaaaccattg gacttactca gaaatgttta aaattacttg aacaactaaa agatagtgtc
gatgattata cacttagcct atatcggcaa ctcatagtcg aattgagaat cagactgaat gttgaattcc aagaaactta
tccggaatta atcggtaaga gtgagaaaga taaagaaaaa gatgcgaaaa ataaacgggc agacaagcgt tacccgcaaa
tttttaagga tataaaatta atccccaatt ttctcggtga aacgaaacaa atggtatata agaaatttat tcgttccgct gacatccttt
atgaaggaat aaattttatc gaccagatcg ataaacagat tactcaaaat ttgttgcctt gttttaagaa cgacaaggaa
cggattgaat ttaccgaaaa acaatttgaa actttacggc gaaaatacta tctgatgaat agttcccgtt ttcaccatgt
tattgaagga ataatcaata ataggaaact tattgaaatg aaaaagagag aaaatagcga gttgaaaact ttctccgata
gtaagtttgt tttatctaag ctttttctta aaaaaggcaa aaaatatgaa aatgaggtct attatacttt ttatataaat ccgaaagctc
gtgaccagcg acggataaaa attgttcttg atataaatgg gaacaattca gtcggaattt tacaagatct tgtccaaaag
ttgaaaccaa aatgggacga catcataaag aaaaatgata tgggagaatt aatcgatgca atcgagattg agaaagtccg
gctcggcatc ttgatagcgt tatactgtga gcataaattc aaaattaaaa aagaactctt gtcattagat ttgtttgcca gtgcctatca
atatctagaa ttggaagatg accctgaaga actttctggg acaaacctag gtcggttttt acaatccttg gtctgctccg
aaattaaagg tgcgattaat aaaataagca ggacagaata tatagagcgg tatactgtcc agccgatgaa tacggagaaa
aactatcctt tactcatcaa taaggaggga aaagccactt ggcatattgc tgctaaggat gacttgtcca agaagaaggg
tgggggcact gtcgctatga atcaaaaaat cggcaagaat tttttaggga aacaagatta taaaactgtg tttatgcttc
aggataagcg gtttgatcta ctaacctcaa agtatcactt gcagttttta tctaaaactc ttgatactgg tggagggtct
tggtggaaaa acaaaaatat tgatttaaat ttaagctctt attctttcat tttcgaacaa aaagtaaaag tcgaatggga tttaaccaat
cttgaccatc ctataaagat taagcctagc gagaacagtg atgatagaag gcttttcgta tccattcctt ttgttattaa
accgaaacag acaaaaagaa aggatttgca aactcgagtc aattatatgg ggattgatat cggagaatat ggtttggctt
ggacaattat taatattgat ttaaagaata aaaaaataaa taagatttca aaacaaggtt tcatctatga gccgttgaca
cataaagtgc gcgattatgt tgctaccatt aaagataatc aggttagagg aacttttggc atgcctgata cgaaactagc
cagattgcga gaaaatgcca ttaccagctt gcgcaatcaa gtgcatgata ttgctatgcg ctatgacgcc aaaccggtat
atgaatttga aatttccaat tttgaaacgg ggtctaataa agtgaaagta atttatgatt cggttaagcg agctgatatc
ggccgaggcc agaataatac cgaagcagac aatactgagg ttaatcttgt ctgggggaag acaagcaaac aatttggcag
tcaaatcggc gcttatgcga caagttacat ctgttcattt tgtggttatt ctccatatta tgaatttgaa aattctaagt cgggagatga
agaaggggct agagataatc tatatcagat gaagaaattg agtcgcccct ctcttgaaga tttcctccaa ggaaatccgg
tttataagac atttagggat tttgataagt ataaaaacga tcaacggttg caaaagacgg gtgataaaga tggtgaatgg
aaaacacaca gagggaatac tgcaatatac gcctgtcaaa agtgtagaca tatctctgat gcggatatcc aagcatcata
ttggattgct ttgaagcaag ttgtaagaga tttttataaa gacaaagaga tggatggtga tttgattcaa ggagataata
aagacaagag aaaagtaaac gagcttaata gacttattgg agtacataaa gatgtgccta taataaataa aaatttaata
acatcactcg acataaactt actataga
CasY.3 Candidatus vogelbacteria amino acid sequence 1200 aa (SEQ ID
NO: 5):
MKAKKSFYNQKRKFGKRGYRLHDERIAYSGGIGSMRSIKYELKDSYGI
AGLRNRIADATISDNKWLYGNINLNDYLEWRSSKTDKQIEDGDRESSLLGFWLEALR
LGFVFSKQSHAPNDFNETALQDLFETLDDDLKHVLDRKKWCDFIKIGTPKTNDQGRL
KKQIKNLLKGNKREEIEKTLNESDDELKEKINRIADVFAKNKSDKYTIFKLDKPNTEK
YPRINDVQVAFFCHPDFEEITERDRTKTLDLIINRFNKRYEITENKKDDKTSNRMALY
SLNQGYIPRVLNDLFLFVKDNEDDFSQFLSDLENFFSFSNEQIKIIKERLKKLKKYAEPI
PGKPQLADKWDDYASDFGGKLESWYSNRIEKLKKIPESVSDLRNNLEKIRNVLKKQ
NNASKILELSQKIIEYIRDYGVSFEKPEIIKFSWINKTKDGQKKVFYVAKMADREFIEK
LDLWMADLRSQLNEYNQDNKVSFKKKGKKIEELGVLDFALNKAKKNKSTKNENG
WQQKLSESIQSAPLFFGEGNRVRNEEVYNLKDLLFSEIKNVENILMSSEAEDLKNIKIE
YKEDGAKKGNYVLNVLARFYARFNEDGYGGWNKVKTVLENIAREAGTDFSKYGN
NNNRNAGRFYLNGRERQVFTLIKFEKSITVEKILELVKLPSLLDEAYRDLVNENKNH
KLRDVIQLSKTIMALVLSHSDKEKQIGGNYIHSKLSGYNALISKRDFISRYSVQTTNGT
QCKLAIGKGKSKKGNEIDRYFYAFQFFKNDDSKINLKVIKNNSHKNIDFNDNENKIN
ALQVYSSNYQIQFLDWFFEKHQGKKTSLEVGGSFTIAEKSLTIDWSGSNPRVGFKRS
DTEEKRVFVSQPFTLIPDDEDKERRKERMIKTKNRFIGIDIGEYGLAWSLIEVDNGDK
NNRGIRQLESGFITDNQQQVLKKNVKSWRQNQIRQTFTSPDTKIARLRESLIGSYKNQ
LESLMVAKKANLSFEYEVSGFEVGGKRVAKIYDSIKRGSVRKKDNNSQNDQSWGK
KGINEWSFETTAAGTSQFCTHCKRWSSLAIVDIEEYELKDYNDNLFKVKINDGEVRL
LGKKGWRSGEKIKGKELFGPVKDAMRPNVDGLGMKIVKRKYLKLDLRDWVSRYG
NMAIFICPYVDCHHISHADKQAAFNIAVRGYLKSVNPDRAIKHGDKGLSRDFLCQEE
GKLNFEQIGLL
CasY.3 Candidatus vogelbacteria nucleic acid sequence (SEQ ID NO: 6):
                                atgaaa gctaaaaaaa glattataa tcaaaagcgg aagttcggta aaagaggtta tcgtcttcac
gatgaacgta tcgcgtattc aggagggatt ggatcgatgc gatctattaa atatgaattg aaggattcgt atggaattgc
tgggcttcgt aatcgaatcg ctgacgcaac tatttctgat aataagtggc tgtacgggaa tataaatcta aatgattatt
tagagtggcg atcttcaaag actgacaaac agattgaaga cggagaccga gaatcatcac tcctgggttt ttggctggaa
gcgttacgac tgggattcgt gttttcaaaa caatctcatg ctccgaatga ttttaacgag accgctctac aagatttgtt tgaaactctt
gatgatgatt tgaaacatgt tcttgatagg aaaaaatggt gtgactttat caagatagga acacctaaga caaatgacca
aggtcgttta aaaaaacaaa tcaagaattt gttaaaagga aacaagagag aggaaattga aaaaactctc aatgaatcag
acgatgaatt gaaagagaaa ataaacagaa ttgccgatgt ttttgcaaaa aataagtctg ataaatacac aattttcaaa
ttagataaac ccaatacgga aaaatacccc agaatcaacg atgttcaggt ggcgtttttt tgtcatcccg attttgagga
aattacagaa cgagatagaa caaagactct agatctgatc attaatcggt ttaataagag atatgaaatt accgaaaata
aaaaagatga caaaacttca aacaggatgg ccttgtattc cttgaaccag ggctatattc ctcgcgtcct gaatgattta ttcttgtttg
tcaaagacaa tgaggatgat tttagtcagt ttttatctga tttggagaat ttcttctctt tttccaacga acaaattaaa ataataaagg
aaaggttaaa aaaacttaaa aaatatgctg aaccaattcc cggaaagccg caacttgctg ataaatggga cgattatgct
tctgattttg gcggtaaatt ggaaagctgg tactccaatc gaatagagaa attaaagaag attccggaaa gcgtttccga
tctgcggaat aatttggaaa agatacgcaa tgttttaaaa aaacaaaata atgcatctaa aatcctggag ttatctcaaa
agatcattga atacatcaga gattatggag tttcttttga aaagccggag ataattaagt tcagctggat aaataagacg
aaggatggtc agaaaaaagt tttctatgtt gcgaaaatgg cggatagaga attcatagaa aagcttgatt tatggatggc
tgatttacgc agtcaattaa atgaatacaa tcaagataat aaagtttctt tcaaaaagaa aggtaaaaaa atagaagagc
tcggtgtctt ggattttgct cttaataaag cgaaaaaaaa taaaagtaca aaaaatgaaa atggctggca acaaaaattg
tcagaatcta ttcaatctgc cccgttattt tttggcgaag ggaatcgtgt acgaaatgaa gaagtttata atttgaagga ccttctgttt
tcagaaatca agaatgttga aaatatttta atgagctcgg aagcggaaga cttaaaaaat ataaaaattg aatataaaga
agatggcgcg aaaaaaggga actatgtctt gaatgtcttg gctagatttt acgcgagatt caatgaggat ggctatggtg
gttggaacaa agtaaaaacc gttttggaaa atattgcccg agaggcgggg actgattttt caaaatatgg aaataataac
aatagaaatg ccggcagatt ttatctaaac ggccgcgaac gacaagtttt tactctaatc aagtttgaaa aaagtatcac
ggtggaaaaa atacttgaat tggtaaaatt acctagccta cttgatgaag cgtatagaga tttagtcaac gaaaataaaa
atcataaatt acgcgacgta attcaattga gcaagacaat tatggctctg gttttatctc attctgataa agaaaaacaa
attggaggaa attatatcca tagtaaattg agcggataca atgcgcttat ttcaaagcga gattttatct cgcggtatag
cgtgcaaacg accaacggaa ctcaatgtaa attagccata ggaaaaggca aaagcaaaaa aggtaatgaa attgacaggt
atttctacgc ttttcaattt tttaagaatg acgacagcaa aattaattta aaggtaatca aaaataattc gcataaaaac atcgatttca
acgacaatga aaataaaatt aacgcattgc aagtgtattc atcaaactat cagattcaat tcttagactg gttttttgaa
aaacatcaag ggaagaaaac atcgctcgag gtcggcggat cttttaccat cgccgaaaag agtttgacaa tagactggtc
ggggagtaat ccgagagtcg gttttaaaag aagcgacacg gaagaaaaga gggtttttgt ctcgcaacca tttacattaa
taccagacga tgaagacaaa gagcgtcgta aagaaagaat gataaagacg aaaaaccgtt ttatcggtat cgatatcggt
gaatatggtc tggcttggag tctaatcgaa gtggacaatg gagataaaaa taatagagga attagacaac ttgagagcgg
ttttattaca gacaatcagc agcaagtctt aaagaaaaac gtaaaatcct ggaggcaaaa ccaaattcgt caaacgttta
cttcaccaga cacaaaaatt gctcgtcttc gtgaaagttt gatcggaagt tacaaaaatc aactggaaag tctgatggtt
gctaaaaaag caaatcttag ttttgaatac gaagtttccg ggtttgaagt tgggggaaag agggttgcaa aaatatacga
tagtataaag cgtgggtcgg tgcgtaaaaa ggataataac tcacaaaatg atcaaagttg gggtaaaaag ggaattaatg
agtggtcatt cgagacgacg gctgccggaa catcgcaatt ttgtactcat tgcaagcggt ggagcagttt agcgatagta
gatattgaag aatatgaatt aaaagattac aacgataatt tatttaaggt aaaaattaat gatggtgaag ttcgtctcct tggtaagaaa
ggttggagat ccggcgaaaa gatcaaaggg aaagaattat ttggtcccgt caaagacgca atgcgcccaa atgttgacgg
actagggatg aaaattgtaa aaagaaaata tctaaaactt gatctccgcg attgggtttc aagatatggg aatatggcta
ttttcatctg tccttatgtc gattgccacc atatctctca tgcggataaa caagctgctt ttaatattgc cgtgcgaggg tatttgaaaa
gcgttaatcc tgacagagca ataaaacacg gagataaagg tttgtctagg gactttttgt gccaagaaga gggtaagctt
aattttgaac aaatagggtt attatgaa
CasY.4 Candidatus parcubacteria amino acid sequence 1210 aa (SEQ ID
NO: 7):
MSKRHPRISGVKGYRLHAQRLEYTGKSGAMRTIKYPLYSSPSGGRTVP
REIVSAINDDYVGLYGLSNFDDLYNAEKRNEEKVYSVLDFWYDCVQYGAVFSYTAP
GLLKNVAEVRGGSYELTKTLKGSHLYDELQIDKVIKFLNKKEISRANGSLDKLKKDII
DCFKAEYRERHKDQCNKLADDIKNAKKDAGASLGERQKKLFRDFFGISEQSENDKP
SFTNPLNLTCCLLPFDTVNNNRNRGEVLFNKLKEYAQKLDKNEGSLEMWEYIGIGNS
GTAFSNFLGEGFLGRLRENKITELKKAMMDITDAWRGQEQEEELEKRLRILAALTIKL
REPKFDNHWGGYRSDINGKLSSWLQNYINQTVKIKEDLKGHKKDLKKAKEMINRFG
ESDTKEEAVVSSLLESIEKIVPDDSADDEKPDIPAIAIYRRFLSDGRLTLNRFVQREDV
QEALIKERLEAEKKKKPKKRKKKSDAEDEKETIDFKELFPHLAKPLKLVPNFYGDSK
RELYKKYKNAAIYTDALWKAVEKIYKSAFSSSLKNSFFDTDFDKDFFIKRLQKIFSVY
RRFNTDKWKPIVKNSFAPYCDIVSLAENEVLYKPKQSRSRKSAAIDKNRVRLPSTENI
AKAGIALARELSVAGFDWKDLLKKEEHEEYIDLIELHKTALALLLAVTETQLDISALD
FVENGTVKDFMKTRDGNLVLEGRFLEMFSQSIVFSELRGLAGLMSRKEFITRSAIQT
MNGKQAELLYIPHEFQSAKITTPKEMSRAFLDLAPAEFATSLEPESLSEKSLLKLKQM
RYYPHYFGYELTRTGQGIDGGVAENALRLEKSPVKKREIKCKQYKTLGRGQNKIVL
YVRSSYYQTQFLEWFLHRPKNVQTDVAVSGSFLIDEKKVKTRWNYDALTVALEPVS
GSERVFVSQPFTIFPEKSAEEEGQRYLGIDIGEYGIAYTALEITGDSAKILDQNFISDPQ
LKTLREEVKGLKLDQRRGTFAMPSTKIARIRESLVHSLRNRIHHLALKHKAKIVYELE
VSRFEEGKQKIKKVYATLKKADVYSEIDADKNLQTTVWGKLAVASEISASYTSQFCG
ACKKLWRAEMQVDETITTQELIGTVRVIKGGTLIDAIKDFMRPPIFDENDTPFPKYRD
FCDKHHISKKMRGNSCLFICPFCRANADADIQASQTIALLRYVKEEKKVEDYFERFR
KLKNIKVLGQMKKI
CasY.4 Candidatus parcubacteria nucleic acid sequence (SEQ ID NO: 8):
                      atgagtaagc gacatcctag aattagcggc gtaaaagggt accgtttgca tgcgcaacgg ctggaatata
ccggcaaaag tggggcaatg cgaacgatta aatatcctct ttattcatct ccgagcggtg gaagaacggt tccgcgcgag
atagtttcag caatcaatga tgattatgta gggctgtacg gtttgagtaa ttttgacgat ctgtataatg cggaaaagcg
caacgaagaa aaggtctact cggttttaga tttttggtac gactgcgtcc aatacggcgc ggttttttcg tatacagcgc
cgggtctttt gaaaaatgtt gccgaagttc gcgggggaag ctacgaactt acaaaaacgc ttaaagggag ccatttatat
gatgaattgc aaattgataa agtaattaaa tttttgaata aaaaagaaat ttcgcgagca aacggatcgc ttgataaact
gaagaaagac atcattgatt gcttcaaagc agaatatcgg gaacgacata aagatcaatg caataaactg gctgatgata
ttaaaaatgc aaaaaaagac gcgggagctt ctttagggga gcgtcaaaaa aaattatttc gcgatttttt tggaatttca
gagcagtctg aaaatgataa accgtctttt actaatccgc taaacttaac ctgctgttta ttgccttttg acacagtgaa
taacaacaga aaccgcggcg aagttttgtt taacaagctc aaggaatatg ctcaaaaatt ggataaaaac gaagggtcgc
ttgaaatgtg ggaatatatt ggcatcggga acagcggcac tgccttttct aattttttag gagaagggtt tttgggcaga
ttgcgcgaga ataaaattac agagctgaaa aaagccatga tggatattac agatgcatgg cgtgggcagg aacaggaaga
agagttagaa aaacgtctgc ggatacttgc cgcgcttacc ataaaattgc gcgagccgaa atttgacaac cactggggag
ggtatcgcag tgatataaac ggcaaattat ctagctggct tcagaattac ataaatcaaa cagtcaaaat caaagaggac
ttaaagggac acaaaaagga cctgaaaaaa gcgaaagaga tgataaatag gtttggggaa agcgacacaa aggaagaggc
ggttgtttca tctttgcttg aaagcattga aaaaattgtt cctgatgata gcgctgatga cgagaaaccc gatattccag ctattgctat
ctatcgccgc tttctttcgg atggacgatt aacattgaat cgctttgtcc aaagagaaga tgtgcaagag gcgctgataa
aagaaagatt ggaagcggag aaaaagaaaa aaccgaaaaa gcgaaaaaag aaaagtgacg ctgaagatga aaaagaaaca
attgacttca aggagttatt tcctcatctt gccaaaccat taaaattggt gccaaacttt tacggcgaca gtaagcgtga
gctgtacaag aaatataaga acgccgctat ttatacagat gctctgtgga aagcagtgga aaaaatatac aaaagcgcgt
tctcgtcgtc tctaaaaaat tcattttttg atacagattt tgataaagat ttttttatta agcggcttca gaaaattttt tcggtttatc
gtcggtttaa tacagacaaa tggaaaccga ttgtgaaaaa ctctttcgcg ccctattgcg acatcgtctc acttgcggag
aatgaagttt tgtataaacc gaaacagtcg cgcagtagaa aatctgccgc gattgataaa aacagagtgc gictcccttc
cactgaaaat atcgcaaaag ctggcattgc cctcgcgcgg gagctttcag tcgcaggatt tgactggaaa gatttgttaa
aaaaagagga gcatgaagaa tacattgatc tcatagaatt gcacaaaacc gcgcttgcgc ttcttcttgc cgtaacagaa
acacagcttg acataagcgc gttggatttt gtagaaaatg ggacggtcaa ggattttatg aaaacgcggg acggcaatct
ggttttggaa gggcgtttcc ttgaaatgtt ctcgcagtca attgigittt cagaattgcg cgggcttgcg ggtttaatga
gccgcaagga atttatcact cgctccgcga ttcaaactat gaacggcaaa caggcggagc ttctctacat tccgcatgaa
ttccaatcgg caaaaattac aacgccaaag gaaatgagca gggcgtttct tgaccttgcg cccgcggaat ttgctacatc
gcttgagcca gaatcgcttt cggagaagtc attattgaaa ttgaagcaga tgcggtacta tccgcattat tttggatatg
agcttacgcg aacaggacag gggattgatg gtggagtcgc ggaaaatgcg ttacgacttg agaagtcgcc agtaaaaaaa
cgagagataa aatgcaaaca gtataaaact ttgggacgcg gacaaaataa aatagtgtta tatgtccgca gttcttatta
tcagacgcaa tttttggaat ggtttttgca tcggccgaaa aacgttcaaa ccgatgttgc ggttagcggt tcgtttctta
tcgacgaaaa gaaagtaaaa actcgctgga attatgacgc gcttacagtc gcgcttgaac cagtttccgg aagcgagcgg
gtctttgtct cacagccgtt tactattttt ccggaaaaaa gcgcagagga agaaggacag aggtatcttg gcatagacat
cggcgaatac ggcattgcgt atactgcgct tgagataact ggcgacagtg caaagattct tgatcaaaat tttatttcag
acccccagct taaaactctg cgcgaggagg tcaaaggatt aaaacttgac caaaggcgcg ggacatttgc catgccaagc
acgaaaatcg cccgcatccg cgaaagcctt gtgcatagtt tgcggaaccg catacatcat cttgcgttaa agcacaaagc
aaagattgtg tatgaattgg aagtgtcgcg ttttgaagag ggaaagcaaa aaattaagaa agtctacgct acgttaaaaa
aagcggatgt gtattcagaa attgacgcgg ataaaaattt acaaacgaca gtatggggaa aattggccgt tgcaagcgaa
atcagcgcaa gctatacaag ccagttttgt ggtgcgtgta aaaaattgtg gcgggcggaa atgcaggttg acgaaacaat
tacaacccaa gaactaatcg gcacagttag agtcataaaa gggggcactc ttattgacgc gataaaggat tttatgcgcc
cgccgatttt tgacgaaaat gacactccat ttccaaaata tagagacttt tgcgacaagc atcacatttc caaaaaaatg
cgtggaaaca gctgtttgtt catttgtcca ttctgccgcg caaacgcgga tgctgatatt caagcaagcc aaacaattgc
gcttttaagg tatgttaagg aagagaaaaa ggtagaggac tactttgaac gatttagaaa gctaaaaaac attaaagtgc
tcggacagat gaagaaaata tgatag
CasY.5 Candidatus komeilibacteria amino acid sequence 1192 aa (SEQ ID
NO: 9):
MAESKQMQCRKCGASMKYEVIGLGKKSCRYMCPDCGNHTSARKIQN
KKKRDKKYGSASKAQSQRIAVAGALYPDKKVQTIKTYKYPADLNGEVHDRGVAEK
IEQAIQEDEIGLLGPSSEYACWIASQKQSEPYSVVDFWFDAVCAGGVFAYSGARLLST
VLQLSGEESVLRAALASSPFVDDINLAQAEKFLAVSRRTGQDKLGKRIGECFAEGRL
EALGIKDRMREFVQAIDVAQTAGQRFAAKLKIFGISQMPEAKQWNNDSGLTVCILPD
YYVPEENRADQLVVLLRRLREIAYCMGIEDEAGFEHLGIDPGALSNFSNGNPKRGFL
GRLLNNDIIALANNMSAMTPYWEGRKGELIERLAWLKHRAEGLYLKEPHFGNSWA
DHRSRIFSRIAGWLSGCAGKLKIAKDQISGVRTDLFLLKRLLDAVPQSAPSPDFIASIS
ALDRFLEAAESSQDPAEQVRALYAFHLNAPAVRSIANKAVQRSDSQEWLIKELDAV
DHLEFNKAFPFFSDTGKKKKKGANSNGAPSEEEYTETESIQQPEDAEQEVNGQEGNG
ASKNQKKFQRIPRFFGEGSRSEYRILTEAPQYFDMFCNNMRAIFMQLESQPRKAPRDF
KCFLQNRLQKLYKQTFLNARSNKCRALLESVLISWGEFYTYGANEKKFRLRHEASER
SSDPDYVVQQALEIARRLFLFGFEWRDCSAGERVDLVEIHKKAISFLLAITQAEVSVG
SYNWLGNSTVSRYLSVAGTDTLYGTQLEEFLNATVLSQMRGLAIRLSSQELKDGFD
VQLESSCQDNLQHLLVYRASRDLAACKRATCPAELDPKILVLPAGAFIASVMKMIER
GDEPLAGAYLRHRPHSFGWQIRVRGVAEVGMDQGTALAFQKPTESEPFKIKPFSAQY
GPVLWLNSSSYSQSQYLDGFLSQPKNWSMRVLPQAGSVRVEQRVALIWNLQAGKM
RLERSGARAFFMPVPFSFRPSGSGDEAVLAPNRYLGLFPHSGGIEYAVVDVLDSAGF
KILERGTIAVNGFSQKRGERQEEAHREKQRRGISDIGRKKPVQAEVDAANELHRKYT
DVATRLGCRIVVQWAPQPKPGTAPTAQTVYARAVRTEAPRSGNQEDHARMKSSWG
YTWSTYWEKRKPEDILGISTQVYWTGGIGESCPAVAVALLGHIRATSTQTEWEKEEV
VFGRLKKFFPS
CasY.5 Candidatus komeilibacteria nucleic acid sequence (SEQ ID NO:
10):
                          accaaccacc tattgcgtct tatcgctca attagcaaa agtggctgtc tagacataca ggtggaaagg
tgagagtaaa gacatggcct gaatagcgtc ctcgtcctcg tctagacata caggtggaaa ggtgagagta aagaccggag
cactcatcct ctcactctat tttgtctaga catacaggtg gaaaggtgag agtaaagaca aaccgtgcca cactaaaccg
atgagtctag acatacaggt ggaaaggtga gagtaaagac tcaagtaact acctgttctt tcacaagtct agacatacag
gtggaaaggt gagagtaaag actcaagtaa ctacctgttc tttcacaagt ctagacctgc aggtggtaag gtgagagtaa
agactcaagt aactacctgt tctttcacaa gtctagacct gcaggtggta aggtgagagt aaagactttt atcctcctct ctatgcttct
gagtctagac atttaggtgg aaaggtgaga gtaaagactt gtggagatcc atgaacttcg gcagtctaga cctgcaggtg
gaaaggtgag agtaaagacg tccttcacac gatcttcctc tgttagtcta ggcctgcagg tggaaaggtg agagtaaaga
cgcataagcg taattgaagc tctctccggt ccagaccttg tcgcgcttgt gttgcgacaa aggcggagtc cgcaataagt
tctttttaca atgttttttc cataaaaccg atacaatcaa gtatcggttt tgcttttttt atgaaaatat gttatgctat gtgctcaaat
aaaaatatca ataaaatagc gtttttttga taatttatcg ctaaaattat acataatcac gcaacattgc cattctcaca caggagaaaa
gtcatggcag aaagcaagca gatgcaatgc cgcaagtgcg gcgcaagcat gaagtatgaa gtaattggat tgggcaagaa
gtcatgcaga tatatgtgcc cagattgcgg caatcacacc agcgcgcgca agattcagaa caagaaaaag cgcgacaaaa
agtatggatc cgcaagcaaa gcgcagagcc agaggatagc tgtggctggc gcgctttatc cagacaaaaa agtgcagacc
ataaagacct acaaataccc agcggatctg aatggcgaag ttcatgacag aggcgtcgca gagaagattg agcaggcgat
tcaggaagat gagatcggcc tgcttggccc gtccagcgaa tacgcttgct ggattgcttc acaaaaacaa agcgagccgt
attcagttgt agatttttgg tttgacgcgg tgtgcgcagg cggagtattc gcgtattctg gcgcgcgcct gctttccaca
gtcctccagt tgagtggcga ggaaagcgtt ttgcgcgctg ctttagcatc tagcccgttt gtagatgaca ttaatttggc
gcaagcggaa aagttcctag ccgttagccg gcgcacaggc caagataagc taggcaagcg cattggagaa tgtttcgcgg
aaggccggct tgaagcgctt ggcatcaaag atcgcatgcg cgaattcgtg caagcgattg atgtggccca aaccgcgggc
cagcggttcg cggccaagct aaagatattc ggcatcagtc agatgcctga agccaagcaa tggaacaatg attccgggct
cactgtatgt attttgccgg attattatgt cccggaagaa aaccgcgcgg accagctggt tgttttgctt cggcgcttac
gcgagatcgc gtattgcatg ggaattgagg atgaagcagg atttgagcat ctaggcattg accctggcgc tctttccaat
ttttccaatg gcaatccaaa gcgaggattt ctcggccgcc tgctcaataa tgacattata gcgctggcaa acaacatgtc
agccatgacg ccgtattggg aaggcagaaa aggcgagttg attgagcgcc ttgcatggct taaacatcgc gctgaaggat
tgtatttgaa agagccacat ttcggcaact cctgggcaga ccaccgcagc aggattttca gtcgcattgc gggctggctt
tccggatgcg cgggcaagct caagattgcc aaggatcaga tttcaggcgt gcgtacggat ttgtttctgc tcaagcgcct
tctggatgcg gtaccgcaaa gcgcgccgtc gccggacttt attgcttcca tcagcgcgct ggatcggttt ttggaagcgg
cagaaagcag ccaggatccg gcagaacagg tacgcgcttt gtacgcgttt catctgaacg cgcctgcggt ccgatccatc
gccaacaagg cggtacagag gtctgattcc caggagtggc ttatcaagga actggatgct gtagatcacc ttgaattcaa
caaagcattt ccgttttttt cggatacagg aaagaaaaag aagaaaggag cgaatagcaa cggagcgcct tctgaagaag
aatacacgga aacagaatcc attcaacaac cagaagatgc agagcaggaa gtgaatggtc aagaaggaaa tggcgcttca
aagaaccaga aaaagtttca gcgcattcct cgatttttcg gggaagggtc aaggagtgag tatcgaattt taacagaagc
gccgcaatat tttgacatgt tctgcaataa tatgcgcgcg atctttatgc agctagagag tcagccgcgc aaggcgcctc
gtgatttcaa atgctttctg cagaatcgtt tgcagaagct ttacaagcaa acctttctca atgctcgcag taataaatgc
cgcgcgcttc tggaatccgt ccttatttca tggggagaat tttatactta tggcgcgaat gaaaagaagt ttcgtctgcg
ccatgaagcg agcgagcgca gctcggatcc ggactatgtg gttcagcagg cattggaaat cgcgcgccgg cttttcttgt
tcggatttga gtggcgcgat tgctctgctg gagagcgcgt ggatttggtt gaaatccaca aaaaagcaat ctcatttttg
cttgcaatca ctcaggccga ggtttcagtt ggttcctata actggcttgg gaatagcacc gtgagccggt atctttcggt
tgctggcaca gacacattgt acggcactca actggaggag tttttgaacg ccacagtgct ttcacagatg cgtgggctgg
cgattcggct ttcatctcag gagttaaaag acggatttga tgttcagttg gagagttcgt gccaggacaa tctccagcat
ctgctggtgt atcgcgcttc gcgcgacttg gctgcgtgca aacgcgctac atgcccggct gaattggatc cgaaaattct
tgttctgccg gctggtgcgt ttatcgcgag cgtaatgaaa atgattgagc gtggcgatga accattagca ggcgcgtatt
tgcgtcatcg gccgcattca ttcggctggc agatacgggt tcgtggagtg gcggaagtag gcatggatca gggcacagcg
ctagcattcc agaagccgac tgaatcagag ccgtttaaaa taaagccgtt ttccgctcaa tacggcccag tactttggct
taattcttca tcctatagcc agagccagta tctggatgga tttttaagcc agccaaagaa ttggtctatg cgggtgctac
ctcaagccgg atcagtgcgc gtggaacagc gcgttgctct gatatggaat ttgcaggcag gcaagatgcg gctggagcgc
tctggagcgc gcgcgttttt catgccagtg ccattcagct tcaggccgtc tggttcagga gatgaagcag tattggcgcc
gaatcggtac ttgggacttt ttccgcattc cggaggaata gaatacgcgg tggtggatgt attagattcc gcgggtttca
aaattcttga gcgcggtacg attgcggtaa atggcttttc ccagaagcgc ggcgaacgcc aagaggaggc acacagagaa
aaacagagac gcggaatttc tgatataggc cgcaagaagc cggtgcaagc tgaagttgac gcagccaatg aattgcaccg
caaatacacc gatgttgcca ctcgtttagg gtgcagaatt gtggttcagt gggcgcccca gccaaagccg ggcacagcgc
cgaccgcgca aacagtatac gcgcgcgcag tgcggaccga agcgccgcga tctggaaatc aagaggatca tgctcgtatg
aaatcctctt ggggatatac ctggagcacc tattgggaga agcgcaaacc agaggatatt ttgggcatct caacccaagt
atactggacc ggcggtatag gcgagtcatg tcccgcagtc gcggttgcgc ttttggggca cattagggca acatccactc
aaactgaatg ggaaaaagag gaggttgtat tcggtcgact gaagaagttc tttccaagct agacgatctt tttaaaaact
gggctgctgg ctatcgtatg gtcagtagct cttattatt tacttgatat atggtattat
CasY.6 Candidatus kerfeldbacteria amino acid sequence 1287 aa (SEQ ID
NO: 11):
MKRILNSLKVAALRLLFRGKGSELVKTVKYPLVSPVQGAVEELAEAIR
HDNLHLFGQKEIVDLMEKDEGTQVYSVVDFWLDTLRLGMFFSPSANALKITLGKFN
SDQVSPFRKVLEQSPFFLAGRLKVEPAERILSVEIRKIGKRENRVENYAADVETCFIGQ
LSSDEKQSIQKLANDIWDSKDHEEQRMLKADFFAIPLIKDPKAVTEEDPENETAGKQ
KPLELCVCLVPELYTRGFGSIADFLVQRLTLLRDKMSTDTAEDCLEYVGIEEEKGNG
MNSLLGTFLKNLQGDGFEQIFQFMLGSYVGWQGKEDVLRERLDLLAEKVKRLPKPK
FAGEWSGHRMFLHGQLKSWSSNFFRLFNETRELLESIKSDIQHATMLISYVEEKGGY
HPQLLSQYRKLMEQLPALRTKVLDPEIEMTHMSEAVRSYIMIHKSVAGFLPDLLESL
DRDKDREFLLSIFPRIPKIDKKTKEIVAWELPGEPEEGYLFTANNLFRNFLENPKHVPR
FMAERIPEDWTRLRSAPVWFDGMVKQWQKVVNQLVESPGALYQFNESFLRQRLQA
MLTVYKRDLQTEKFLKLLADVCRPLVDFFGLGGNDIIFKSCQDPRKQWQTVIPLSVP
ADVYTACEGLAIRLRETLGFEWKNLKGHEREDFLRLHQLLGNLLFWIRDAKLVVKL
EDWMNNPCVQEYVEARKAIDLPLEIFGFEVPIFLNGYLFSELRQLELLLRRKSVMTSY
SVKTTGSPNRLFQLVYLPLNPSDPEKKNSNNFQERLDTPTGLSRRFLDLTLDAFAGKL
LTDPVTQELKTMAGFYDHLFGFKLPCKLAAMSNHPGSSSKMVVLAKPKKGVASNIG
FEPIPDPAHPVFRVRSSWPELKYLEGLLYLPEDTPLTIELAETSVSCQSVSSVAFDLKN
LTTILGRVGEFRVTADQPFKLTPIIPEKEESFIGKTYLGLDAGERSGVGFAIVTVDGDG
YEVQRLGVHEDTQLMALQQVASKSLKEPVFQPLRKGTFRQQERIRKSLRGCYWNFY
HALMIKYRAKVVHEESVGSSGLVGQWLRAFQKDLKKADVLPKKGGKNGVDKKKR
ESSAQDTLWGGAFSKKEEQQIAFEVQAAGSSQFCLKCGWWFQLGMREVNRVQESG
VVLDWNRSIVTFLIESSGEKVYGFSPQQLEKGFRPDIETFKKMVRDFMRPPMFDRKG
RPAAAYERFVLGRRHRRYRFDKVFEERFGRSALFICPRVGCGNFDHSSEQSAVVLALI
GYIADKEGMSGKKLVYVRLAELMAEWKLKKLERSRVEEQSSAQ
CasY.6 Candidatus kerfeldbacteria nucleic acid sequence (SEQ ID NO:
12):
                          atgaagag aattctgaac agtctgaaag ttgctgcctt gagacttctg tttcgaggca aaggttctga
attagtgaag acagtcaaat atccattggt ttccccggtt caaggcgcgg ttgaagaact tgctgaagca attcggcacg
acaacctgca ccttttaggg cagaaggaaa tagtggatct tatggagaaa gacgaaggaa cccaggtgta ttcggttgtg
gatttttggt tggataccct gcgtttaggg atgtttttct caccatcagc gaatgcgttg aaaatcacgc tgggaaaatt caattctgat
caggtttcac cttttcgtaa ggttttggag cagtcacctt tttttcttgc gggtcgcttg aaggttgaac ctgcggaaag gatactttct
gttgaaatca gaaagattgg taaaagagaa aacagagttg agaactatgc cgccgatgtg gagacatgct tcattggtca
gctttcttca gatgagaaac agagtatcca gaagctggca aatgatatct gggatagcaa ggatcatgag gaacagagaa
tgttgaaggc ggattttttt gctatacctc ttataaaaga ccccaaagct gtcacagaag aagatcctga aaatgaaacg
gcgggaaaac agaaaccgct tgaattatgt gtttgtcttg ttcctgagtt gtatacccga ggtttcggct ccattgctga tatctggtt
cagcgactta ccttgctgcg tgacaaaatg agtaccgaca cggcggaaga ttgcctcgag tatgttggca ttgaggaaga
aaaaggcaat ggaatgaatt ccttgctcgg cacttttttg aagaacctgc agggtgatgg ttttgaacag atttttcagt ttatgcttgg
gtcttatgtt ggctggcagg ggaaggaaga tgtactgcgc gaacgattgg atttgctggc cgaaaaagtc aaaagattac
caaagccaaa atttgccgga gaatggagtg gtcatcgtat gtttctccat ggtcagctga aaagctggtc gtcgaatttc
ttccgtcttt ttaatgagac gcgggaactt ctggaaagta tcaagagtga tattcaacat gccaccatgc tcattagcta
tgtggaagag aaaggaggct atcatccaca gctgttgagt cagtatcgga agttaatgga acaattaccg gcgttgcgga
ctaaggtttt ggatcctgag attgagatga cgcatatgtc cgaggctgtt cgaagttaca ttatgataca caagtctgta
gcgggatttc tgccggattt actcgagtct ttggatcgag ataaggatag ggaattttag ctttccatct ttcctcgtat tccaaagata
gataagaaga cgaaagagat cgttgcatgg gagctaccgg gcgagccaga ggaaggctat ttgttcacag caaacaacct
tttccggaat tttcttgaga atccgaaaca tgtgccacga tttatggcag agaggattcc cgaggattgg acgcgtttgc
gctcggcccc tgtgtggttt gatgggatgg tgaagcaatg gcagaaggtg gtgaatcagt tggttgaatc tccaggcgcc
ctttatcagt tcaatgaaag ttttttgcgt caaagactgc aagcaatgct tacggtctat aagcgggatc tccagactga
gaagtttctg aagctgctgg ctgatgtctg tcgtccactc gttgattttt tcggacttgg aggaaatgat attatcttca agtcatgtca
ggatccaaga aagcaatggc agactgttat tccactcagt gtcccagcgg atgtttatac agcatgtgaa ggcttggcta
ttcgtctccg cgaaactctt ggattcgaat ggaaaaatct gaaaggacac gagcgggaag attttttacg gctgcatcag
ttgctgggaa atctgctgtt ctggatcagg gatgcgaaac ttgtcgtgaa gctggaagac tggatgaaca atccttgtgt
tcaggagtat gtggaagcac gaaaagccat tgatcttccc ttggagattt tcggatttga ggtgccgatt tttctcaatg gctatctctt
ttcggaactg cgccagctgg aattgttgct gaggcgtaag tcggtgatga cgtcttacag cgtcaaaacg acaggctcgc
caaataggct cttccagttg gtttacctac ctctaaaccc ttcagatccg gaaaagaaaa attccaacaa ctttcaggag
cgcctcgata cacctaccgg tttgtcgcgt cgttttctgg atcttacgct ggatgcattt gctggcaaac tcttgacgga
tccggtaact caggaactga agacgatggc cggtttttac gatcatctct ttggcttcaa gttgccgtgt aaactggcgg
cgatgagtaa ccatccagga tcctcttcca aaatggtggt tctggcaaaa ccaaagaagg gtgttgctag taacatcggc
tttgaaccta ttcccgatcc tgctcatcct gtgttccggg tgagaagttc ctggccggag ttgaagtacc tggaggggtt
gttgtatctt cccgaagata caccactgac cattgaactg gcggaaacgt cggtcagttg tcagtctgtg agttcagtcg
ctttcgattt gaagaatctg acgactatct tgggtcgtgt tggtgaattc agggtgacgg cagatcaacc tttcaagctg
acgcccatta ttcctgagaa agaggaatcc ttcatcggga agacctacct cggtcttgat gctggagagc gatctggcgt
tggtttcgcg attgtgacgg ttgacggcga tgggtatgag gtgcagaggt tgggtgtgca tgaagatact cagcttatgg
cgcttcagca agtcgccagc aagtctctta aggagccggt tttccagcca ctccgtaagg gcacatttcg tcagcaggag
cgcattcgca aaagcctccg cggttgctac tggaatttct atcatgcatt gatgatcaag taccgagcta aagttgtgca
tgaggaatcg gtgggttcat ccggtctggt ggggcagtgg ctgcgtgcat ttcagaagga tctcaaaaag gctgatgttc
tgcccaagaa gggtggaaaa aatggtgtag acaaaaaaaa gagagaaagc agcgctcagg ataccttatg gggaggagct
ttctcgaaga aggaagagca gcagatagcc tttgaggttc aggcagctgg atcaagccag ttttgtctga agtgtggttg
gtggtttcag ttggggatgc gggaagtaaa tcgtgtgcag gagagtggcg tggtgctgga ctggaaccgg tccattgtaa
ccttcctcat cgaatcctca ggagaaaagg tatatggttt cagtcctcag caactggaaa aaggctttcg tcctgacatc
gaaacgttca aaaaaatggt aagggatttt atgagacccc ccatgtttga tcgcaaaggt cggccggccg cggcgtatga
aagattcgta ctgggacgtc gtcaccgtcg ttatcgcttt gataaagttt ttgaagagag atttggtcgc agtgctcttt tcatctgccc
gcgggtcggg tgtgggaatt tcgatcactc cagtgagcag tcagccgttg tccttgccct tattggttac attgctgata
aggaagggat gagtggtaag aagcttgttt atgtgaggct ggctgaactt atggctgagt ggaagctgaa gaaactggag
agatcaaggg tggaagaaca gagctcggca caataa
CasX.1 Planctomycetes amino acid sequence 978 aa (SEQ ID NO: 13):
MQEIKRINKIRRRLVKDSNTKKAGKTGPMKTLLVRVMTPDLRERLENLRKK
PENIPQPISNTSRANLNKLLTDYTEMKKAILHVYWEEFQKDPVGLMSRVAQPAPKNI
DQRKLIPVKDGNERLTSSGFACSQCCQPLYVYKLEQVNDKGKPHTNYFGRCNVSEH
ERLILLSPHKPEANDELVTYSLGKFGQRALDFYSIHVTRESNHPVKPLEQIGGNSCAS
GPVGKALSDACMGAVASFLTKYQDIILEHQKVIKKNEKRLANLKDIASANGLAFPKI
TLPPQPHTKEGIEAYNNVVAQIVIWVNLNLWQKLKIGRDEAKPLQRLKGFPSFPLVE
RQANEVDWWDMVCNVKKLINEKKEDGKVFWQNLAGYKRQEALLPYLSSEEDRKK
GKKFARYQFGDLLLHLEKKHGEDWGKVYDEAWERIDKKVEGLSKHIKLEEERRSED
AQSKAALTDWLRAKASFVIEGLKEADKDEFCRCELKLQKWYGDLRGKPFAIEAENSI
LDISGFSKQYNCAFIWQKDGVKKLNLYLIINYFKGGKLRFKKIKPEAFEANRFYTVIN
KKSGEIVPMEVNFNFDDPNLIILPLAFGKRQGREFIWNDLLSLETGSLKLANGRVIEKT
LYNRRTRQDEPALFVALTFERREVLDSSNIKPMNLIGIDRGENIPAVIALTDPEGCPLS
RFKDSLGNPTHILRIGESYKEKQRTIQAAKEVEQRRAGGYSRKYASKAKNLADDMV
RNTARDLLYYAVTQDAMLIFENLSRGFGRQGKRTFMAERQYTRMEDWLTAKLAYE
GLPSKTYLSKTLAQYTSKTCSNCGFTITSADYDRVLEKLKKTATGWMTTINGKELKV
EGQITYYNRYKRQNVVKDLSVELDRLSEESVNNDISSWTKGRSGEALSLLKKRFSHR
PVQEKFVCLNCGFETHADEQAALNIARSWLFLRSQEYKKYQTNKTTGNTDKRAFVE
TWQSFYRKKLKEVWKPAV
CasX.1 Planctomycetes nucleic acid sequence (SEQ ID NO: 14):
          atgct tcttatttat cggagatatc ttcaaacacc atcaacatgg caatggtgaa ccattaatat tctttgatgc
ttcttattta tcggagatat cttcaaacat tgcccatttt acaggcatat cttctggctc tttgatgctt cttatttatc ggagatatct
tcaaacgtaa tgtattgaga aagacatcaa gattagataa ctttgatgct tcttatttat cggagatatc ttcaaacaca
gaaacctgca aagattgtat atatataagc tttgatgctt cttatttatc ggagatatct tcaaacgata cgtattttag cccgtctatt
tggggattaa ctttgatgct tcttatttat cggagatatc ttcaaacccc gcatatccag atttttcaat gacttctgga aattgtattt
tcaatatttt acaagttgcg gaggatacct ttaataattt agcagagtta cgcactgtaa acctgttctt ctcacaaaaa gctttaacat
cagattttca aagaacttct tatgtaattt ataagaatct aaaaaaacag ctctgggttt gcatccagaa ctctccgata
aataagcgct ttacccatac gacatagtcg ctggtgatgg ctctcaaagt aatgagataa aagcgccagt aataatttac
tattcacaaa tcctttcgtc aagcttaaaa tcaatcaaag accatatccc cttcattcca aatagcagcg cttccgtacc tttctatccg
ttcatatatc tcctctgaga gaggataaat taccagactt atagagccat ccataaatcc tttttcttta aggttgagct ttagatcagc
ccaccttgct tttgaaaggt taaactcaaa gacagaatat tgaatccgaa caccataggc ttccagaagt ttaactaacc
gtgccctgac cttatcatct tcaatatcat aacaaatgag atgtcgcatt ttaaagctct ataggcttat aacattccct atcatcttga
atatgctggc taaacaacct aacctgccgc tcaactgcgt gctgatacgt tattgattgg ataagtaaat tggttttctg ctcatctacc
ttaaagaatt gatgccattt tttgattact tttggatagg catccttatt cagccaaaca cctttttggt cagtttcttt cctgaaatcg
tctgtatcca cttcccttct atttatcaaa ttgatcacaa aacggtcagc caacggccgc cactcctcca gaagatcgca
tattaaagag ggacgaccat aatagacgtc atgcaagtaa ccaaaggccg ggtcaaaacc gacgagtaat gcagtcgaat
gtatttcgtt gaacaggagg gtgtagataa ggctcatcat ggcgttgatt tcatcctcag gaggtctctt ggtacggcgc
acaaaaacaa agcttggatg ctttaagata gccgaaaaat tgccataata ctgccttgtt gttgcgcctt ctattccacg
caaggtctct aaatcagtga cggcgttgat ttcggtacac tcgattctca aaccaagtct atatttatca agtaatgatt gctggttttt
gatcttaccg gcaacgatac tttttgcaat ttcaagtttt ttgtggggat caaaatgctt atgaatttgc gcccgacgaa taaacagatt
tttgacgggt tcaaattgaa ggctcccttg atattcccat ctgccgctaa agaaatgtat cggtatagat tattctctgc
aaaggctaat aacacggcta tcgagggtaa cccggccaac taccacgata tcttttacct tcattgcggg aatcttctgc
cccttctctt cattgtcctt ttttatgaga aatgcccgac cacgacaatc caaaatgaat tcatcacccg tgagatagag
ggttatcctg tcggttatag cggtcatcag taagcctttt atttttctaa ccaagtattg aaggaagaca cgattcacta tactggcact
gcggacacct atggtcatca accttgggaa acctgcttat atcaaaggac aagaagcagt ctcgcagatt tgtaacaact
tctacacaac gcactttcag ggttttatct ataacaattt ctttccgtct ccgtgtttca cagaaaaata tttcaccaac tggtatattg
acattataca tctcttcaag gcaaattgcc tgtaacccaa tctgaacgtg gaagttctca aaatccctta ccttccctgt ctttgtttcg
ataggaatcg gtatcccatc cctccactcg ataaggtctg cccggcctgc caaaccgagc ttattgctgt aaagatacac
gcctgttacc tgcttacaat cagggcagct tctctgcgat gatttatcca ccgccctgtg cgcgtgtatg gcctctgtaa
agtggatgct cttagccata ttacgccgtt ctccaacaaa ggcataccat gcattgcgcg gacaatagat tgactccatt
accgtgctga tgtgcaatat cagacggctg gtttccatac ttctttgagc ttctttctgt aaaaggattg ccatgtttca acaaatgccc
ttttgtcagt atttccggtc gttttattgg tttgatacttcttatattct tgagaacgga gaaagagcca cgaccttgca atattcagtg
ctgcttgttc gtctgcatgg gtttcaaaac cacagttcag gcaaacaaac ttttcctgca ccggcctgtg actaaatctc atatagca
gagataaagc ttcaccactg cggccttttg tccaactaga aatatcatta tttaccgact cttccgaaag tctatccagc
tctacagaga ggtcttttac cacattctgc cttttatacc ggttatagta tgttatctgt ccttcaactt ttaactcttt tccattgatt
gtagtcatcc atccagtagc cgtcttcttg agcttttcga gcaccctgtc ataatctgca cttgtgattg taaaaccaca
attagaacat gtctttgagg tatactgtgc cagagtcttt gaaagatagg tttttgatgg cagaccttca taggcaagct
ttgcagtcag ccagtcttcc atcctcgtgt actgcctttc cgccataaaa gtcctcttgc cttgtctacc aaaaccgcgg
gaaagatttt caaaaatgag cattgcatct tgagtaacag cataatataa gaggtcacga gctgtatttc ttaccatatc
gtccgccaga ttcttcgcct ttgatgcata ttttctcgaa tatccgcctg cccgcctttg ttcaacttct ttagcagcct gaatagtccg
ttgtttttcc ttataacttt ctcctattcg caaaatatgc gttggattgc ccaatgaatc tttgaatctt gacaaggggc atccttccgg
gtctgttaat gctatgactg ccgggatatt ttctccccgg tctattccta tcagattcat cggttttata ttcgatgagt caagcacctc
tcttctttca aatgtcaggg caacaaaaag tgctggttca tcctgtctcg tccttctgtt atagagcgtt ttttcaataa ccctgccatt
ggcgagtttc aatgaacccg tctcaaggct caataggtcg ttccagataa actccctccc ctgccttttt ccaaaggcca
aaggcagaat tatcaaattc gggtcatcaa aattgaagtt gacctccata ggcacaatct caccgctttt tttattaatt actgtataaa
acctatttgc ttcaaaagct tctggcttga tttttttgaa gcgtagctta ccacctttga agtaatttat tattaaataa agatttaact
tctttacgcc gtctttctgc catataaatg cacaattata ctgtttagaa aatccgctta tatctaaaat gctgttctct gcttctatag
caaatggttt tcctctcaaa tctccatacc acttttgaag ctttaactca cacctgcaaa actcatcctt atcagcttct ttgagccctt
caataacaaa agaggccttt gccctgagcc aatcagtgag ggcagccttt gattgagcat cttcagacct tctttcttcc
tccaacttta tgtgcttact cagaccttca acttttttat ctattctttc ccatgcctca tcataaactt tgccccaatc ttcaccgtgt
ttcttttcaa ggtgaagcaa aaggtcacca aactgataac gcgcaaactt ttttcctttt ttacggtctt cttcagacga aagatatgga
agcaaggctt cctgcctttt atatccagca agattttgcc agaagacctt cccgtcctct ttcttttcgt taatcaactt tttgacatta
cagaccatat cccaccaatc aacctcattc gcctggcgtt caacaagagg gaaggacgga aaacccttaa gccgctgtaa
gggctttgcc tcatccctgc caattttgag tttctgccaa agattcaggt ttacccagat cactatctga gcaacaacat
tgttataagc ttcaatccct tcttttgtat gcggttgcgg tggaagagtg attttaggaa atgcaagccc gtttgcactt gctatatcct
ttagatttgc caatctcttt tcgttttttt ttataacctt ttggtgttcg aggatgatgt cctggtactt tgtaaggaaa ctggctactg
ctcccataca ggcatcagat aaagccttac caacgggacc acttgcgcag ctattgccac cgatctgttc tagcggcttt
acaggatggt tcgattctct tgttacgtgg attgaataaa agtccaatgc cctttgaccg aacttcccca acgaatacgt
tactagctcg tcatttgcct ccggtttatg cggcgagagc aatatcaaac gttcatgctc ggagacatta caacggccaa
agtaatttgt atggggctta cccttgtcat tcacttgttc aagcttataa acatagaggg gttgacagca ctgagaacag
gcaaatccag aacttgttag tctctcattt ccgtccttca ccggaatcaa ttttctctga tcaatattct tgggcgctgg ttgtgcaacc
ctgctcatca atccgacagg gtctttttgg aactcttccc aataaacatg caggattgct ttcttcattt ccgtatagtc agtgaggagt
ttatttaaat ttgcacgtga agtatttgaa atgggctgag gaatgttttc cggctttttg cgaagattct ctaacctttc tctcaggtca
ggtgtcataa cccgaacgag caaggttttc atagggccgg ttttgccggc ttttttcgtg ttgctatcct ttaccaatct ccttcgtatt
ttatttatcc tttttatttc ctgcatcttt
CasX.1 Deltaproteobacteria amino acid sequence 986 aa (SEQ ID NO: 15):
MEKRINKIRKKLSADNATKPVSRSGPMKTLLVRVMTDDLKKRLEKRR
KKPEVMPQVISNNAANNLRMLLDDYTKMKEAILQVYWQEFKDDHVGLMCKFAQP
ASKKIDQNKLKPEMDEKGNLTTAGFACSQCGQPLFVYKLEQVSEKGKAYTNYFGRC
NVAEHEKLILLAQLKPEKDSDEAVTYSLGKFGQRALDFYSIHVTKESTHPVKPLAQIA
GNRYASGPVGKALSDACMGTIASFLSKYQDIIIEHQKVVKGNQKRLESLRELAGKEN
LEYPSVTLPPQPHTKEGVDAYNEVIARVRMWVNLNLWQKLKLSRDDAKPLLRLKGF
PSFPVVERRENEVDWWNTINEVKKLIDAKRDMGRVFWSGVTAEKRNTILEGYNYLP
NENDHKKREGSLENPKKPAKRQFGDLLLYLEKKYAGDWGKVFDEAWERIDKKIAG
LTSHIEREEARNAEDAQSKAVLTDWLRAKASFVLERLKEMDEKEFYACEIQLQKWY
GDLRGNPFAVEAENRVVDISGFSIGSDGHSIQYRNLLAWKYLENGKREFYLLMNYG
KKGRIRFTDGTDIKKSGKWQGLLYGGGKAKVIDLTFDPDDEQLIILPLAFGTRQGREF
IWNDLLSLETGLIKLANGRVIEKTIYNKKIGRDEPALFVALTFERREVVDPSNIKPVNL
IGVDRGENIPAVIALTDPEGCPLPEFKDSSGGPTDILRIGEGYKEKQRAIQAAKEVEQR
RAGGYSRKFASKSRNLADDMVRNSARDLFYHAVTHDAVLVFENLSRGFGRQGKRT
FMTERQYTKMEDWLTAKLAYEGLTSKTYLSKTLAQYTSKTCSNCGFTITTADYDGM
LVRLKKTSDGWATTLNNKELKAEGQITYYNRYKRQTVEKELSAELDRLSEESGNNDI
SKWTKGRRDEALFLLKKRFSHRPVQEQFVCLDCGHEVHADEQAALNIARSWLFLNS
NSTEFKSYKSGKQPFVGAWQAFYKRRLKEVWKPNA
CasX.1 Deltaproteobacteria nucleic acid sequence (SEQ ID NO: 16):
                                      at ggaaaagaga ataaacaaga tacgaaagaa actatcggcc gataatgcca caaagcctgt
gagcaggagc ggccccatga aaacactcct tgtccgggtc atgacggacg acttgaaaaa aagactggag aagcgtcgga
aaaagccgga agttatgccg caggttattt caaataacgc agcaaacaat cttagaatgc tccttgatga ctatacaaag
atgaaggagg cgatactaca agtttactgg caggaattta aggacgacca tgtgggcttg atgtgcaaat ttgcccagcc
tgcttccaaa aaaattgacc agaacaaact aaaaccggaa atggatgaaa aaggaaatct aacaactgcc ggttttgcat
gttctcaatg cggtcagccg ctatttgttt ataagcttga acaggtgagt gaaaaaggca aggcttatac aaattacttc
ggccggtgta atgtggccga gcatgagaaa ttgattcttc ttgctcaatt aaaacctgaa aaagacagtg acgaagcagt
gacatactcc cttggcaaat tcggccagag ggcattggac ttttattcaa tccacgtaac aaaagaatcc acccatccag
taaagcccct ggcacagatt gcgggcaacc gctatgcaag cggacctgtt ggcaaggccc tttccgatgc ctgtatgggc
actatagcca gttttctttc gaaatatcaa gacatcatca tagaacatca aaaggttgtg aagggtaatc aaaagaggtt
agagagtctc agggaattgg cagggaaaga aaatcttgag tacccatcgg ttacactgcc gccgcagccg catacgaaag
aaggggttga cgcttataac gaagttattg caagggtacg tatgtgggtt aatcttaatc tgtggcaaaa gctgaagctc
agccgtgatg acgcaaaacc gctactgcgg ctaaaaggat tcccatcttt ccctgttgtg gagcggcgtg aaaacgaagt
tgactggtgg aatacgatta atgaagtaaa aaaactgatt gacgctaaac gagatatggg acgggtattc tggagcggcg
ttaccgcaga aaagagaaat accatccttg aaggatacaa ctatctgcca aatgagaatg accataaaaa gagagagggc
agtttggaaa accctaagaa gcctgccaaa cgccagtttg gagacctctt gctgtatctt gaaaagaaat atgccggaga
ctggggaaag gtcttcgatg aggcatggga gaggatagat aagaaaatag ccggactcac aagccatata gagcgcgaag
aagcaagaaa cgcggaagac gctcaatcca aagccgtact tacagactgg ctaagggcaa aggcatcatt tgttcttgaa
agactgaagg aaatggatga aaaggaattc tatgcgtgtg aaatccaact tcaaaaatgg tatggcgatc ttcgaggcaa
cccgtttgcc gttgaagctg agaatagagt tgttgatata agcgggtttt ctatcggaag cgatggccat tcaatccaat
acagaaatct ccttgcctgg aaatatctgg agaacggcaa gcgtgaattc tatctgttaa tgaattatgg caagaaaggg
cgcatcagat ttacagatgg aacagatatt aaaaagagcg gcaaatggca gggactatta tatggcggtg gcaaggcaaa
ggttattgat ctgactttcg accccgatga tgaacagttg ataatcctgc cgctggcctt tggcacaagg caaggccgcg
agtttatctg gaacgatttg ctgagtcttg aaacaggcct gataaagctc gcaaacggaa gagttatcga aaaaacaatc
tataacaaaa aaatagggcg ggatgaaccg gctctattcg ttgccttaac atttgagcgc cgggaagttg ttgatccatc
aaatataaag cctgtaaacc ttataggcgt tgaccgcggc gaaaacatcc cggcggttat tgcattgaca gaccctgaag
gttgtccttt accggaattc aaggattcat cagggggccc aacagacatc ctgcgaatag gagaaggata taaggaaaag
cagagggcta ttcaggcagc aaaggaggta gagcaaaggc gggctggcgg ttattcacgg aagtttgcat ccaagtcgag
gaacctggcg gacgacatgg tgagaaattc agcgcgagac cttttttacc atgccgttac ccacgatgcc gtccttgtct
ttgaaaacct gagcaggggt tttggaaggc agggcaaaag gaccttcatg acggaaagac aatatacaaa gatggaagac
tggctgacag cgaagctcgc atacgaaggt cttacgtcaa aaacctacct ttcaaagacg ctggcgcaat atacgtcaaa
aacatgctcc aactgcgggt ttactataac gactgccgat tatgacggga tgttggtaag gcttaaaaag acttctgatg
gatgggcaac taccctcaac aacaaagaat taaaagccga aggccagata acgtattata accggtataa aaggcaaacc
gtggaaaaag aactctccgc agagcttgac aggctttcag aagagtcggg caataatgat atttctaagt ggaccaaggg
tcgccgggac gaggcattat ttttgttaaa gaaaagattc agccatcggc ctgttcagga acagtttgtt tgcctcgatt
gcggccatga agtccacgcc gatgaacagg cagccttgaa tattgcaagg tcatggcttt ttctaaactc aaattcaaca
gaattcaaaa gttataaatc gggtaaacag cccttcgttg gtgcttggca ggccttttac aaaaggaggc ttaaagaggt
atggaagccc aacgcctgat
ARMAN1 amino acid sequence 950 aa (SEQ ID NO: 17):
MRDSITAPRYSSALAARIKEFNSAFKLGIDLGTKTGGVALVKDNKVLL
AKTFLDYHKQTLEERRIHRRNRRSRLARRKRIARLRSWILRQKIYGKQLPDPYKIKK
MQLPNGVRKGENWIDLVVSGRDLSPEAFVRAITLIFQKRGQRYEEVAKEIEEMSYKE
FSTHIKALTSVTEEEFTALAAEIERRQDVVDTDKEAERYTQLSELLSKVSESKSESKD
RAQRKEDLGKVVNAFCSAHRIEDKDKWCKELMKLLDRPVRHARFLNKVLIRCNICD
RATPKKSRPDVRELLYFDTVRNFLKAGRVEQNPDVISYYKKIYMDAEVIRVKILNKE
KLTDEDKKQKRKLASELNRYKNKEYVTDAQKKMQEQLKTLLFMKLTGRSRYCMA
HLKERAAGKDVEEGLHGVVQKRHDRNIAQRNHDLRVINLIESLLFDQNKSLSDAIRK
NGLMYVTIEAPEPKTKHAKKGAAVVRDPRKLKEKLFDDQNGVCIYTGLQLDKLEIS
KYEKDHIFPDSRDGPSIRDNLVLTTKEINSDKGDRTPWEWMHDNPEKWKAFERRVA
EFYKKGRINERKRELLLNKGTEYPGDNPTELARGGARVNNFITEFNDRLKTHGVQEL
QTIFERNKPIVQVVRGEETQRLRRQWNALNQNFIPLKDRAMSFNHAEDAAIAASMPP
KFWREQIYRTAWHFGPSGNERPDFALAELAPQWNDFFMTKGGPIIAVLGKTKYSWK
HSIIDDTIYKPFSKSAYYVGIYKKPNAITSNAIKVLRPKLLNGEHTMSKNAKYYHQKI
GNERFLMKSQKGGSIITVKPHDGPEKVLQISPTYECAVLTKHDGKIIVKFKPIKPLRD
MYARGVIKAMDKELETSLSSMSKHAKYKELHTHDIIYLPATKKHVDGYFIITKLSAK
HGIKALPESMVKVKYTQIGSENNSEVKLTKPKPEITLDSEDITNIYNFTR
ARMAN1 nucleic acid sequence (SEQ ID NO: 18):
            atga gagactctat tactgcacct agatacagct ccgctcttgc cgccagaata aaggagttta attctgcttt
caagttagga atcgacctag gaacaaaaac cggcggcgta gcactggtaa aagacaacaa agtgctgctc gctaagacat
tcctcgatta ccataaacaa acactggagg aaaggaggat ccatagaaga aacagaagga gcaggctagc caggcggaag
aggattgctc ggctgcgatc atggatactc agacagaaga tttatggcaa gcagcttcct gacccataca aaatcaaaaa
aatgcagttg cctaatggtg tacgaaaagg ggaaaactgg attgacctgg tagtttctgg acgggacctt tcaccagaag
ccttcgtgcg tgcaataact ctgatattcc aaaagagagg gcaaagatat gaagaagtgg ccaaagagat agaagaaatg
agttacaagg aatttagtac tcacataaaa gccctgacat ccgttactga agaagaattt actgctctgg cagcagagat
agaacggagg caggatgtgg ttgacacaga caaggaggcc gaacgctata cccaattgtc tgagttgctc tccaaggtct
cagaaagcaa atctgaatct aaagacagag cgcagcgtaa ggaggatctc ggaaaggtgg tgaacgcttt ctgcagtgct
catcgtatcg aagacaagga taaatggtgt aaagaactta tgaaattact agacagacca gtcagacacg ctaggttcct
taacaaagta ctgatacgtt gcaatatctg cgatagggca acccctaaga aatccagacc tgacgtgagg gaactgctat
attttgacac agtaagaaac ttcttgaagg ctggaagagt ggagcaaaac ccagacgtta ttagttacta taaaaaaatt
tatatggatg cagaagtaat cagggtcaaa attctgaata aggaaaagct gactgatgag gacaaaaagc aaaagaggaa
attagcgagc gaacttaaca ggtacaaaaa caaagaatac gtgactgatg cgcagaagaa gatgcaagag caacttaaga
cattgctgtt catgaagctg acaggcaggt ctagatactg catggctcat cttaaggaaa gggcagcagg caaagatgta
gaagaaggac ttcatggcgt tgtgcagaaa agacacgaca ggaacatagc acagcgcaat cacgacttac gtgtgattaa
tcttattgag agtctgcttt tcgaccaaaa caaatcgctc tccgatgcaa taaggaagaa cgggttaatg tatgttacta
ttgaggctcc agagccaaag actaagcacg caaagaaagg cgcagctgtg gtaagggatc ccagaaagtt gaaggagaag
ttgtttgatg atcaaaacgg cgtttgcata tatacgggct tgcagttaga caaattagag ataagtaaat acgagaagga
ccatatcttt ccagattcaa gggatggacc atctatcagg gacaatcttg tactcactac aaaagagata aattcagaca
aaggcgatag gaccccatgg gaatggatgc atgataaccc agaaaaatgg aaagcgttcg agagaagagt cgcagaattc
tataagaaag gcagaataaa tgagaggaaa agagaactcc tattaaacaa aggcactgaa taccctggcg ataacccgac
tgagctggcg cggggaggcg cccgtgttaa caactttatt actgaattta atgaccgcct caaaacgcat ggagtccagg
aactgcagac catctttgag cgtaacaaac caatagtgca ggtagtcagg ggtgaagaaa cgcagcgtct gcgcagacaa
tggaatgcac taaaccagaa tttcatacca ctaaaggaca gggcaatgtc gttcaaccac gctgaagacg cagccatagc
agcaagcatg ccaccaaaat tctggaggga gcagatatac cgtactgcgt ggcactttgg acctagtgga aatgagagac
cggactttgc tttggcagaa ttggcgccac aatggaatga cttctttatg actaagggcg gtccaataat agcagtgctg
ggcaaaacga agtatagttg gaagcacagc ataattgatg acactatata caagccattc agcaaaagtg cttactatgt
tgggatatac aaaaagccga acgccatcac gtccaatgct ataaaagtct taaggccaaa actcttaaat ggcgaacata
caatgtctaa gaatgcaaag tattatcatc agaagattgg taatgagcgc ttcctcatga aatctcagaa aggtggatcg
ataattacag taaaaccaca cgacggaccg gaaaaagtgc ttcaaatcag ccctacatat gaatgcgcag tccttactaa
gcatgacggt aaaataatag tcaaatttaa accaataaag ccgctacggg acatgtatgc ccgcggtgtg attaaagcca
tggacaaaga gcttgaaaca agcctctcta gcatgagtaa acacgctaag tacaaggagt tacacactca tgatatcata
tatctgcctg ctacaaagaa gcacgtagat ggctacttca taataaccaa actaagtgcg aaacatggca taaaagcact
ccccgaaagc atggttaaag tcaagtatac tcaaattggg agtgaaaaca atagtgaagt gaagcttacc aaaccaaaac
cagagataac tttggatagt gaagatatta caaacatata taatttcacc cgctaag
ARMAN4 amino acid sequence 967 aa (SEQ ID NO: 19):
MLGSSRYLRYNLTSFEGKEPFLIMGYYKEYNKELSSKAQKEFNDQISEFNSY
YKLGIDLGDKTGIAIVKGNKIILAKTLIDLHSQKLDKRREARRNRRTRLSRKKRLARL
RSWVMRQKVGNQRLPDPYKIMHDNKYWSIYNKSNSANKKNWIDLLIHSNSLSADD
FVRGLTIIFRKRGYLAFKYLSRLSDKEFEKYIDNLKPPISKYEYDEDLEELSSRVENGEI
EEKKFEGLKNKLDKIDKESKDFQVKQREEVKKELEDLVDLFAKSVDNKIDKARWKR
ELNNLLDKKVRKIRFDNRFILKCKIKGCNKNTPKKEKVRDFELKMVLNNARSDYQIS
DEDLNSFRNEVINIFQKKENLKKGELKGVTIEDLRKQLNKTFNKAKIKKGIREQIRSIV
FEKISGRSKFCKEHLKEFSEKPAPSDRINYGVNSAREQHDFRVLNFIDKKIFKDKLIDP
SKLRYITIESPEPETEKLEKGQISEKSFETLKEKLAKETGGIDIYTGEKLKKDFEIEHIFP
RARMGPSIRENEVASNLETNKEKADRTPWEWFGQDEKRWSEFEKRVNSLYSKKKIS
ERKREILLNKSNEYPGLNPTELSRIPSTLSDFVESIRKMFVKYGYEEPQTLVQKGKPIIQ
VVRGRDTQALRWRWHALDSNIIPEKDRKSSFNHAEDAVIAACMPPYYLRQKIFREEA
KIKRKVSNKEKEVTRPDMPTKKIAPNWSEFMKTRNEPVIEVIGKVKPSWKNSIMDQT
FYKYLLKPFKDNLIKIPNVKNTYKWIGVNGQTDSLSLPSKVLSISNKKVDSSTVLLVH
DKKGGKRNWVPKSIGGLLVYITPKDGPKRIVQVKPATQGLLIYRNEDGRVDAVREFI
NPVIEMYNNGKLAFVEKENEEELLKYFNLLEKGQKFERIRRYDMITYNSKFYYVTKI
NKNHRVTIQEESKIKAESDKVKSSSGKEYTRKETEELSLQKLAELISI
ARMAN4 nucleic acid sequence (SEQ ID NO: 20):
                at gttaggctcc agcaggtacc tccgttataa cctaacctcg tttgaaggca aggagccatt tttaataatg
ggatattaca aagagtataa taaggaatta agttccaaag ctcaaaaaga atttaatgat caaatttctg aatttaattc gtattacaaa
ctaggtatag atctcggaga taaaacagga attgcaatcg taaagggcaa caaaataatc ctagcaaaaa cactaattga
tttgcattcc caaaaattag ataaaagaag ggaagctaga agaaatagaa gaactcggct ttccagaaag aaaaggcttg
cgagattaag atcgtgggta atgcgtcaga aagttggcaa tcaaagactt cccgatccat ataaaataat gcatgacaat
aagtactggt ctatatataa taagagtaat tctgcaaata aaaagaattg gatagatctg ttaatccaca gtaactcttt
atcagcagac gattttgtta gaggcttaac tataattttc agaaaaagag gctatttagc atttaagtat ctttcaaggt taagcgataa
ggaatttgaa aaatacatag ataacttaaa accacctata agcaaatacg agtatgatga ggatttagaa gaattatcaa
gcagggttga aaatggggaa atagaggaaa agaaattcga aggcttaaag aataagctag ataaaataga caaagaatct
aaagactttc aagtaaagca aagagaagaa gtaaaaaagg aactggaaga cttagttgat ttgtttgcta aatcagttga
taataaaata gataaagcta ggtggaaaag ggagctaaat aatttattgg ataagaaagt aaggaaaata cggtttgaca
accgctttat tttgaagtgc aaaattaagg gctgtaacaa gaatactcca aagaaagaga aggtcagaga ttttgaattg
aagatggttt taaataatgc tagaagcgat tatcagattt ctgatgagga tttaaactct tttagaaatg aagtaataaa tatatttcaa
aagaaggaaa acttaaagaa aggagagctg aaaggagtta ctattgaaga tttgagaaag cagcttaata aaacttttaa
taaagccaag attaaaaaag ggataaggga gcagataagg tctatcgtgt ttgaaaaaat tagtggaagg agtaaattct
gcaaagaaca tctaaaagaa ttttctgaga agccggctcc ttctgacagg attaattatg gggttaattc agcaagagaa
caacatgatt ttagagtctt aaatttcata gataaaaaaa tattcaaaga taagttgata gatccctcaa aattgaggta tataactatt
gaatctccag aaccagaaac agagaagttg gaaaaaggtc aaatatcaga gaagagcttc gaaacattga aagaaaaatt
ggctaaagaa acaggtggta ttgatatata cactggtgaa aaattaaaga aagactttga aatagagcac atattcccaa
gagcaaggat ggggccttct ataagggaaa acgaagtagc atcaaatctg gaaacaaata aggaaaaggc cgatagaact
ccttgggaat ggtttgggca agatgaaaaa agatggtcag agtttgagaa aagagttaat tctctttata gtaaaaagaa
aatatcagag agaaaaagag aaattttgtt aaataagagt aatgaatatc cgggattaaa ccctacagaa ctaagtagaa
tacctagtac gctgagcgac ttcgttgaga gtataagaaa aatgtttgtt aagtatggct atgaagagcc tcaaactttg
gttcaaaaag gaaaaccgat aatacaagtt gttagaggca gagacacaca agctttgagg tggagatggc atgcattaga
tagtaatata ataccagaaa aggacaggaa aagttcattt aatcacgctg aagatgcagt tattgccgcc tgtatgccac
cttactatct caggcaaaaa atatttagag aagaagcaaa aataaaaaga aaagtaagca ataaggaaaa ggaagttaca
cggcctgaca tgcctactaa aaagatagct ccgaactggt cggaatttat gaaaactaga aatgagccgg ttattgaagt
aataggaaaa gttaagccaa gctggaaaaa cagcataatg gatcaaacat tttataaata tcttttgaag ccatttaaag
ataacctgat aaaaataccc aacgttaaaa atacatacaa gtggatagga gttaatggac aaactgattc attatccctc
ccgagtaagg tcttatctat ctctaataaa aaggttgatt cttctacagt tcttcttgtg catgataaga agggtggtaa
gcggaattgg gtacctaaaa gtataggggg tttgttggta tatataactc ctaaagacgg gccgaaaaga atagttcaag
taaagccagc aactcagggt ttgttaatat atagaaatga agatggcaga gtagatgctg taagagagtt cataaatcca
gtgatagaaa tgtataataa tggcaaattg gcatttgtag aaaaagaaaa tgaagaagag cttttgaaat attttaattt
gctggaaaaa ggtcaaaaat ttgaaagaat aagacggtat gatatgataa cctacaatag taaattttac tatgtaacaa
aaataaacaa gaatcacaga gttactatac aagaagagtc taagataaaa gcagaatcag acaaagttaa gtcctcttca
ggcaaagagt atactcgtaa ggaaaccgag gaattatcac ttcaaaaatt agcggaatta attagtatat aaaa
```

TABLE1
Oligonucleotides for gRNAs targeting HIV-1 LTR, Gag and Pol and
PCR primers

| Target | | |
|---|---|---|
| name | Direction | Sequences (5′ to 3′) |
| LTR-A | T353: | aaacAGGGCCAGGGATCAGATATCCACTGACCTTgt |
| | Forward | (SEQ ID NO: 25) |
| | T354: | taaacAAGGTCAGTGGATATCTGATCCCTGGCCCT |
| | Reverse | (SEQ ID NO: 26) |
| LTR-B | T355: | aaacAGCTCGATGTCAGCAGTTCTTGAAGTACTCgt |
| | Forward | (SEQ ID NO: 27) |
| | T356: | taaacGAGTACTTCAAGAACTGCTGACATCGAGCT |
| | Reverse | (SEQ ID NO: 28) |
| LTR-C | T357: | caccGATTGGCAGAACTACACACC (SEQ ID NO: 29) |
| | Forward | |
| | T358: | aaacGGTGTGTAGTTCTGCCAATC (SEQ ID NO: 30) |
| | Reverse | |
| LTR-D | T359: | caccGCGTGGCCTGGGCGGGACTG (SEQ ID NO: 31) |
| | Forward | |
| | T360: | aaacCAGTCCCGCCCAGGCCACGC (SEQ ID NO: 32) |
| | Reverse | |
| LTR-E | T361: | caccGATCTGTGGATCTACCACACACA (SEQ ID NO: |
| | Forward | 33) |
| | T362: | aaacTGTGTGTGGTAGATCCACAGATC (SEQ ID NO: |
| | Reverse | 34) |
| LTR-F | T363: | caccGCTGCTTATATGCAGCATCTGAG (SEQ ID NO: |
| | Forward | 35) |
| | T364: | aaacCTCAGATGCTGCATATAAGCAGC (SEQ ID NO: |
| | Reverse | 36) |
| LTR-G | T530: | caccGTGTGGTAGATCCACAGATCA (SEQ ID NO: |
| | Forward | 37) |
| | T531: | aaacTGATCTGTGGATCTACCACAC (SEQ ID NO: 38) |
| | Reverse | |
| LTR-H | T532 | caccGCAGGGAAGTAGCCTTGTGTG (SEQ ID NO: |
| | Forward | 39) |
| | T533: | aaacCACACAAGGCTACTTCCCTGC (SEQ ID NO: 40) |
| | Reverse | |
| LTR-I | T534: | caccGATCAGATATCCACTGACCTT (SEQ ID NO: 41) |
| | Forward | |
| | T535: | aaacAAGGTCAGTGGATATCTGATC (SEQ ID NO: |
| | Reverse | 42) |
| LTR-J | T536: | caccGCACACTAATACTTCTCCCTC (SEQ ID NO: 43) |
| | Forward | |
| | T537: | aaacGAGGGAGAAGTATTAGTGTGC (SEQ ID NO: |
| | Reverse | 44) |
| LTR-K | T538: | caccGCCTCCTAGCATTTCGTCACA (SEQ ID NO: 45) |
| | Forward | |
| | T539: | aaacTGTGACGAAATGCTAGGAGGC (SEQ ID NO: |
| | Reverse | 46) |
| LTR-L | T540: | caccGCATGGCCCGAGAGCTGCATC (SEQ ID NO: |
| | Forward | 47) |
| | T541: | aaacGATGCAGCTCTCGGGCCATGC (SEQ ID NO: |
| | Reverse | 48) |
| LTR-M | T542: | caccGCAGCAGTCTTTGTAGTACTC (SEQ ID NO: 49) |
| | Forward | |
| | T543: | aaacGAGTACTACAAAGACTGCTGC (SEQ ID NO: |
| | Reverse | 50) |
| LTR-N | T544: | caccGCTGACATCGAGCTTTCTACA (SEQ ID NO: 51) |
| | Forward | |
| | T545: | aaacTGTAGAAAGCTCGATGTCAGC (SEQ ID NO: |
| | Reverse | 52) |
| LTR-O | T546: | caccGTCTACAAGGGACTTTCCGCT (SEQ ID NO: 53) |
| | Forward | |
| | T547: | aaacAGCGGAAAGTCCCTTGTAGAC (SEQ ID NO: |
| | Reverse | 54) |
| LTR-P | T548: | caccGCTTTCCGCTGGGGACTTTCC (SEQ ID NO: 55) |
| | Forward | |
| | T549: | aaacGGAAAGTCCCCAGCGGAAAGC (SEQ ID NO: |
| | Reverse | 56) |
| LTR-Q | T687: | caccGCCTCCCTGGAAAGTCCCCAG (SEQ ID NO: |
| | Forward | 57) |
| | T688: | aaacCTGGGGACTTTCCAGGGAGGC (SEQ ID NO: |
| | Reverse | 58) |
| LTR-R | T689: | caccGCCTGGGCGGGACTGGGGAG (SEQ ID NO: 59) |
| | Forward | |
| | T690: | aaacCTCCCCAGTCCCGCCCAGGC (SEQ ID NO: 60) |
| | Reverse | |
| LTR-S | T691: | caccGTCCATCCCATGCAGGCTCAC (SEQ ID NO: 61) |
| | Forward | |
| | T692: | aaacGTGAGCCTGCATGGGATGGAC (SEQ ID NO: |
| | Reverse | 62) |
| LTR-T | T548: | caccGCGGAGAGAGAAGTATTAGAG (SEQ ID NO: |
| | Forward | 63) |
| | T549: | aaacCTCTAATACTTCTCTCTCCGC (SEQ ID NO: 64) |
| | Reverse | |
| Gag-A | T687: | caccGGCCAGATGAGAGAACCAAG (SEQ ID NO: 65) |
| | Forward | |
| | T688: | aaacCTTGGTTCTCTCATCTGGCC (SEQ ID NO: 66) |
| | Reverse | |
| Gag-B | T714: | caccGCCTTCCCACAAGGGAAGGCCA (SEQ ID NO: |
| | Forward | 67) |
| | T715: | aaacTGGCCTTCCCTTGTGGGAAGGC (SEQ ID NO: |
| | Reverse | 68) |
| Gag-C | T758: | caccGCGAGAGCGTCGGTATTAAGCG (SEQ ID NO: |
| | Forward | 69) |
| | T759: | aaacCGCTTAATACCGACGCTCTCGC (SEQ ID NO: |
| | Reverse | 70) |
| Gag-D | T760: | caccGGATAGATGTAAAAGACACCA (SEQ ID NO: |
| | Forward | 71) |
| | T761: | aaacTGGTGTCTTTTACATCTATCC (SEQ ID NO: 72) |
| | Reverse | |
| Pol-A | T689: | caccGCAGGATATGTAACTGACAG (SEQ ID NO: 73) |
| | Forward | |
| | T690: | aaacCTGTCAGTTACATATCCTGC (SEQ ID NO: 74) |
| | Reverse | |
| Pol-B | T716: | caccGCATGGGTACCAGCACACAA (SEQ ID NO: 75) |
| | Forward | |
| | T717: | aaacTTGTGTGCTGGTACCCATGC (SEQ ID NO: 76) |
| | Reverse | |
| PCR | T422 | caccGCTTTATTGAGGCTTAAGCAG (SEQ ID NO: 77) |
| | T425 | aaacGAGTCACACAACAGACGGGC (SEQ ID NO: 78) |
| | T645 | TGGAATGCAGTGGCGCGATCTTGGC (SEQ ID NO: |
| | | 79) |
| | T477 | CACAGCATCAAGAAGAACCTGAT (SEQ ID NO: 80) |
| | T478 | TGAAGATCTCTTGCAGATAGCAG (SEQ ID NO: 81) |

What is claimed is:

1. A composition for preventing or treating a retroviral infection in vitro or in vivo, the composition comprising at least two isolated nucleic acid sequences wherein the first isolated nucleic acid sequences encodes a first Clustered Regularly Interspaced Short Palindromic Repeat (CRISPR)-associated endonuclease and at least one guide RNA (gRNA), the gRNA being complementary to a target sequence in the integrated retroviral DNA; the second isolated nucleic acid sequences encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo.

2. The composition of claim 1, wherein the first isolated nucleic acid sequences encodes at least one gRNA, the gRNA being complementary to a target sequence in the integrated retroviral DNA and a second gRNA that is complementary to a second target sequence in the integrated retroviral DNA.

3. The composition of claim 1, wherein the second isolated nucleic acid sequence encodes a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell.

4. The composition of claims 1-3, wherein the first isolated nucleic acid sequence encodes a first gRNA, the gRNA being complementary to a target sequence in the integrated retroviral DNA and a second gRNA that is complementary to a target sequence in a gene encoding for at least one receptor used by a retrovirus for attachment and/or infection of a cell.

5. The composition of claim 4, wherein the at least one receptor comprises CD4, CXCR4, CXCRS, variants or combinations thereof.

6. The composition of any one of claims 1-5, wherein the first and second isolated nucleic acid sequences encode combinations of gRNAs having complementarity to one or more target sequences, the target sequences comprising retroviral DNA sequences, and sequences in one or more genes encoding for at least one receptor used by a retrovirus for attachment and/or infection of a cell.

7. The composition of any one of claims 1-6, wherein the target sequence comprises one or more nucleic acid sequences in coding and non-coding nucleic acid sequences of the retrovirus genome.

8. The composition of claim 7, wherein the target sequences comprise one or more nucleic acid sequences in HIV comprising: long terminal repeat (LTR) nucleic acid sequences, nucleic acid sequences encoding structural proteins, non-structural proteins or combinations thereof.

9. The composition of claim 7, wherein the sequences encoding structural proteins comprise nucleic acid sequences encoding: Gag, Gag-Pol precursor, Pro (protease), Reverse Transcriptase (RT), integrase (In), Env or combinations thereof.

10. The composition of claim 7, wherein the sequences encoding non-structural proteins comprise nucleic acid sequences encoding: regulatory proteins, accessory proteins or combinations thereof.

11. The composition of claim 7, wherein regulatory proteins comprise: Tat, Rev or combinations thereof.

12. The composition of claim 7, wherein accessory proteins comprise Nef, Vpr, Vpu, Vif or combinations thereof.

13. The composition of any one of claims 1-12, wherein said gRNA target sequences comprise one or more target sequences in an LTR region of an HIV proviral DNA and one or more target sequences in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

14. The composition of any one of claims 1-13, wherein a gRNA has a 60% sequence identity to any one or more of a gRNA has a 60% sequence identity to any one or more of SEQ ID NOS: 21-114 and to one or more target sequences of SEQ ID NOS: 115 and 116.

15. The composition of any one of claims 1-14, wherein a gRNA comprises SEQ ID NOS: 21-114 and to one or more target sequences of SEQ ID NOS: 115 and 116.

16. The composition of any one of claims 1-15, wherein a gRNA has a 60% sequence identity to any one or more of SEQ ID NOS: 21-24.

17. The composition of any one of claims 1-16, wherein a gRNA comprises SEQ ID NOS: 21-24.

18. The composition of any one of claims 1-17, wherein the first Clustered Regularly Interspaced Short Palindromic Repeat (CRISPR)-associated endonuclease and at least one guide RNA (gRNA) at least one gRNA comprising SEQ ID NOS: 25-116; wherein the second Clustered Regularly Interspaced Short Palindromic Repeat (CRISPR)-associated endonuclease and at least one guide RNA (gRNA) comprising SEQ ID NOS: 21-24.

19. The composition of claim 1, wherein the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof.

20. The composition of claim 1, wherein a nucleic acid encoding for the endonuclease has at least a 60% sequence identity to any one or more of SEQ ID NOS: 1 to 20.

21. The composition of claim 1, wherein a nucleic acid encoding for the endonuclease comprises any one or more of SEQ ID NOS: 1 to 20.

22. An isolated nucleic acid sequence encoding a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo.

23. The isolated nucleic acid of claim 22, wherein the isolated nucleic acid sequence further comprises two or more gRNAs complementary to a target sequence in the integrated retroviral DNA; and/or two or more gRNAs complementary to a target sequence in a gene encoding for at least one receptor used by a retrovirus for attachment and/or infection of a cell in vitro or in vivo.

24. The isolated nucleic acid of claim 22, wherein the at least one receptor comprises CD4, CXCR4, CXCR5, variants or combinations thereof.

25. The isolated nucleic acid of claim 22, wherein the isolated nucleic acid sequence further comprises a combination of one or more gRNAs complementary to a target sequence in the integrated retroviral DNA and/or a one or more gRNAs complementary to a target sequence in a gene encoding for at least one receptor used by a retrovirus for attachment and/or infection of a cell in vitro or in vivo.

26. The isolated nucleic acid of claim 22, wherein the isolated nucleic acid sequence further comprises two or more gRNAs complementary to a target sequence in the integrated retroviral DNA and/or two or more gRNAs complementary to a target sequence in a gene encoding for at least one receptor used by a retrovirus for attachment and/or infection of a cell in vitro or in vivo.

27. The isolated nucleic acid of claim 22, wherein a gRNA has at least a 60% sequence identity to any one or more of SEQ ID NOS: 21-24.

28. The isolated nucleic acid of claim 22, wherein a gRNA comprises SEQ ID NOS: 21-24.

29. The isolated nucleic acid of claim 22, wherein the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof.

30. The isolated nucleic acid of claim 22, wherein a nucleic acid encoding for the endonuclease has at least a 60% sequence identity to any one or more of SEQ ID NOS: 1 to 20.

31. The isolated nucleic acid of claim 22, wherein a nucleic acid encoding for the endonuclease comprises any one or more of SEQ ID NOS: 1 to 20.

32. A method of inactivating an integrated retroviral DNA and preventing infection by a retrovirus in vitro or in vivo, including the steps of exposing the cell to a composition comprising at least one isolated nucleic acid sequence encoding a gene editing complex comprising a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo.

33. The method of claim 32, wherein the integrated retroviral DNA is HIV-1 DNA, the at least one isolated nucleic acid sequence encodes a first gRNA complementary to a target nucleic acid sequence in an LTR region of the HIV-1 DNA.

34. The method of claim 32, further comprising a gRNA complementary to a target nucleic acid sequence in a structural gene or LTR region of the HIV DNA.

35. The method of claim 32, wherein the second guide RNA is complementary to a target sequence encoding for at least one receptor used by a retrovirus for attachment and/or infection of a cell, said at least one receptor comprises CD4, CXCR4, CXCR5, variants or combinations thereof.

36. The method of claim 32, wherein a gRNA has a 60% sequence identity to any one or more of a gRNA has a 60% sequence identity to any one or more of SEQ ID NOS: 21-114 and to one or more target sequences of SEQ ID NOS: 115 and 116.

37. The method of claim 32, wherein a gRNA comprises SEQ ID NOS: 21-114 and to one or more target sequences of SEQ ID NOS: 115 and 116.

38. The method of claim 32, wherein a gRNA has a 60% sequence identity to any one or more of SEQ ID NOS: 21-24.

39. The method of claim 32, wherein a gRNA comprises SEQ ID NOS: 21-24.

40. A pharmaceutical composition comprising at least two isolated nucleic acid sequences wherein the first isolated nucleic acid sequences encodes a first Clustered Regularly Interspaced Short Palindromic Repeat (CRISPR)-associated endonuclease and at least one guide RNA (gRNA), the gRNA being complementary to a target sequence in the integrated retroviral DNA; the second isolated nucleic acid sequences encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo.

41. A pharmaceutical composition comprising isolated nucleic acid sequence encoding a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo.

42. The pharmaceutical composition of claim 41, wherein the isolated nucleic acid sequence further comprises two or more gRNAs complementary to a target sequence in the integrated retroviral DNA; and/or two or more gRNAs complementary to a target sequence in a gene encoding for at least one receptor used by a retrovirus for attachment and/or infection of a cell in vitro or in vivo.

43. An expression vector comprising a first isolated nucleic acid sequence encoding a Clustered Regularly Interspaced Short Palindromic Repeat (CRISPR)-associated endonuclease and at least one guide RNA (gRNA), the gRNA being complementary to a target sequence in a proviral DNA for, inactivating a proviral DNA integrated into the genome of a host cell latently infected with a retrovirus, and a second isolated nucleic acid sequences encoding a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo.

44. The expression vector of claim 43, further comprising two or more gRNAs, wherein a gRNA includes at least a first guide gRNA that is complementary to a first target sequence in a proviral DNA; a second gRNA that is complementary to a second target sequence in the proviral DNA, a third and/or fourth gRNA, said third and fourth gRNAs being complementary to a third and foruth target sequence in a gene encoding for at least one receptor used by a retrovirus for attachment and/or infection of a cell in vitro or in vivo.
